(19) United States
(12) Patent Application Publication
(10) Pub. No.: US 2025/0236613 A1
(43) Pub. Date: Jul. 24, 2025

(54) SARM1 MODULATORS, PREPARATIONS, AND USES THEREOF

(71) Applicant: SIRONAX LTD., Grand Cayman (KY)

(72) Inventors: Zhaolan ZHANG, Beijing (CN); Zhiyuan ZHANG, Beijing (CN); Weidong SUN, Beijing (CN); Yanping XU, Beijing (CN); Yimin JIANG, Beijing (CN); Lianzhu LIU, Beijing (CN)

(21) Appl. No.: 18/699,315

(22) PCT Filed: Oct. 25, 2022
(86) PCT No.: PCT/CN2022/127233

§ 371 Date: Apr. 8, 2024

(30) Foreign Application Priority Data
Oct. 25, 2021 (WO) PCT/CN2021/125941

(51) Int. Cl. C07D 413/04; A61K 31/4418; A61K 31/4439; A61K 31/444; A61K 31/4709; A61K 31/4725; A61K 31/501; A61K 31/506; A61K 31/517; A61K 31/536; A61K 31/538; A61K 31/553; C07D 213/30; C07D 213/34; C07D 213/38; C07D 213/57; C07D 213/60; C07D 401/04; C07D 413/14; C07D 417/04; C07D 417/14; C07D 471/04

(52) U.S. Cl. CPC C07D 413/04; A61K 31/4418; A61K 31/4439; A61K 31/444; A61K 31/4709; A61K 31/4725; A61K 31/501; A61K 31/506; A61K 31/517; A61K 31/536; A61K 31/538; A61K 31/553; C07D 213/30; C07D 213/34; C07D 213/38; C07D 213/57; C07D 213/60; C07D 401/04; C07D 413/14; C07D 417/04; C07D 417/14; C07D 471/04

(57) ABSTRACT

Provided herein are compounds of Formula (I), compositions comprising the same, and methods of using the same, including use in treating various diseases and conditions, e.g., those caused by axonal degeneration.

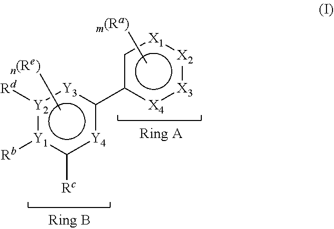

### FIELD OF THE DISCLOSURE

The present disclosure relates to compounds that modulate SARM1, compositions comprising the compounds, methods of preparing the compounds, and methods of using the compounds to treat various diseases or conditions, e.g., those caused by axonal degeneration.

### BACKGROUND OF THE DISCLOSURE

Axonal degeneration causes disease progression and accumulation of disability in many degenerative diseases of the peripheral nervous system (PNS) and central nervous systems (CNS), such as multiple sclerosis, Parkinson's disease, and amyotrophic lateral sclerosis (ALS), or acute conditions such as traumatic brain injury. (Hughes R O, Bosanac T, Mao X, Engber T M, DiAntonio A, Milbrandt J, Devraj R, Krauss R. Small Molecule SARM1 Inhibitors Recapitulate the SARM1−/−Phenotype and Allow Recovery of a Metastable Pool of Axons Fated to Degenerate. Cell Rep. 2021 Jan. 5; 34(1):108588. doi: 10.1016/j.celrep.2020.108588. PMID: 33406435; PMCID: PMC8179325) (Todd Bosanac, Robert O Hughes, Thomas Engber, Rajesh Devraj, Andrew Brearley, Kerstin Danker, Kenneth Young, Jens Kopatz, Melanie Hermann, Antoine Berthemy, Susan Boyce, Jonathan Bentley, Raul Krauss, Pharmacological SARM1 inhibition protects axon structure and function in paclitaxel-induced peripheral neuropathy, Brain, 2021, awab184, https://doi.org/10.1093/brain/awab184)). Therefore, axonal protection is an important neuroprotective approach to treatment in chronic and acute CNS and PNS neurodegenerative disorders. (Hughes R O, Bosanac T, Mao X, Engber T M, DiAntonio A, Milbrandt J, Devraj R, Krauss R. Small Molecule SARM1 Inhibitors Recapitulate the SARM1−/− Phenotype and Allow Recovery of a Metastable Pool of Axons Fated to Degenerate. Cell Rep. 2021 Jan. 5; 34(1):108588. doi: 10.1016/j.celrep.2020.108588. PMID: 33406435; PMCID: PMC8179325) (Todd Bosanac, Robert O Hughes, Thomas Engber, Rajesh Devraj, Andrew Brearley, Kerstin Danker, Kenneth Young, Jens Kopatz, Melanie Hermann, Antoine Berthemy, Susan Boyce, Jonathan Bentley, Raul Krauss, Pharmacological SARM1 inhibition protects axon structure and function in paclitaxel-induced peripheral neuropathy, Brain, 2021, awab184, https://doi.org/10.1093/brain/awab184).

SARM1 (Sterile Alpha and TIR Motif-containing 1) is a unique member of the Myd88 family of adaptor proteins and is considered a major driver of an evolutionarily conserved program of axonal degeneration downstream of chemical, inflammatory, mechanical or metabolic insults to the axon. (Hughes R O, Bosanac T, Mao X, Engber T M, DiAntonio A, Milbrandt J, Devraj R, Krauss R. Small Molecule SARM1 Inhibitors Recapitulate the SARM1−/− Phenotype and Allow Recovery of a Metastable Pool of Axons Fated to Degenerate. Cell Rep. 2021 Jan. 5; 34(1):108588. doi: 10.1016/j.celrep.2020.108588. PMID: 33406435; PMCID: PMC8179325.) (Bosanac T, Hughes R O, Engber T, Devraj R, Brearley A, Danker K, Young K, Kopatz J, Hermann M, Berthemy A, Boyce S, Bentley J, Krauss R. Pharmacological SARM1 inhibition protects axon structure and function in paclitaxel-induced peripheral neuropathy. Brain. 2021 May 8:awab184. doi: 10.1093/brain/awab184. Epub ahead of print. PMID: 33964142). SARM1 has been recognized as a central mediator of axonal degeneration in a number of diseases or conditions, including ALS, Parkinson's disease, multiple sclerosis, traumatic brain injury, and diabetic neuropathy, as well as chemotherapy induced peripheral neuropathy (CIPN), which is a major cause of morbidity and the main cause of dose reductions and discontinuations in cancer treatment. (Hughes R O, Bosanac T, Mao X, Engber T M, DiAntonio A, Milbrandt J, Devraj R, Krauss R. Small Molecule SARM1 Inhibitors Recapitulate the SARM1−/− Phenotype and Allow Recovery of a Metastable Pool of Axons Fated to Degenerate. Cell Rep. 2021 Jan. 5; 34(1):108588. doi: 10.1016/j.celrep.2020.108588. PMID: 33406435; PMCID: PMC8179325) (Bosanac T, Hughes R O, Engber T, Devraj R, Brearley A, Danker K, Young K, Kopatz J, Hermann M, Berthemy A, Boyce S, Bentley J, Krauss R. Pharmacological SARM1 inhibition protects axon structure and function in paclitaxel-induced peripheral neuropathy. Brain. 2021 May 8:awab184. doi: 10.1093/brain/awab184. Epub ahead of print. PMID: 33964142).

SARM1 is a compelling target to treat neurodegeneration characterized by axonopathies of the peripheral and central nervous systems SARM1 contains a mitochondrial targeting sequence, an N-terminal domain with armadillo repeats (ARM), two sterile α-motif (SAM) domains, and a Toll/interleukin-1 receptor (TIR) domain (Gerdts J, Summers D W, Sasaki Y, DiAntonio A, Milbrandt J. Sarml-mediated axon degeneration requires both SAM and TIR interactions. J Neurosci. 2013 Aug. 14; 33(33):13569-80. doi: 10.1523/JNEUROSCI.1197-13.2013. PMID: 23946415; PMCID: PMC3742939.) The SARM1 TIR domain is a NAD+hydrolases (NADase), which convert the NAD+ to ADPR or cADPR and NAM (Sporny M, Guez-Haddad J, Lebendiker M, Ulisse V, Volf A, Mim C, Isupov M N, Opatowsky Y. Structural Evidence for an Octameric Ring Arrangement of SARM1. J Mol Biol. 2019 Sep. 6; 431(19):3591-3605. doi: 10.1016/j.jmb.2019.06.030. Epub 2019 Jul. 3. PMID: 31278906). This NADase activity is essential for its axonal degenerative function. (Bosanac T, Hughes R O, Engber T, Devraj R, Brearley A, Danker K, Young K, Kopatz J, Hermann M, Berthemy A, Boyce S, Bentley J, Krauss R. Pharmacological SARM1 inhibition protects axon structure and function in paclitaxel-induced peripheral neuropathy. Brain. 2021 May 8:awab184. doi: 10.1093/brain/awab184. Epub ahead of print. PMID: 33964142). The activity of SARM1 also depends on the oligomerization formed through SAM domain (Sporny M, Guez-Haddad J, Lebendiker M, Ulisse V, Volf A, Mim C, Isupov M N, Opatowsky Y. Structural Evidence for an Octameric Ring Arrangement of SARM1. J Mol Biol. 2019 Sep. 6; 431(19):3591-3605. doi: 10.1016/j.jmb.2019.06.030. Epub 2019 Jul. 3. PMID: 31278906) and is autoinhibited by the ARM domain (Shen C, Vohra M, Zhang P, Mao X, Figley M D, Zhu J, Sasaki Y, Wu H, DiAntonio A, Milbrandt J. Multiple domain interfaces mediate SARM1 autoinhibition. Proc Natl Acad Sci USA. 2021 Jan. 26; 118(4):e2023151118. doi: 10.1073/pnas.2023151118. PMID: 33468661; PMCID: PMC7848697).

Certain SARM1 inhibitors are disclosed by Bosanac et al. (Bosanac T, Hughes R O, Engber T, Devraj R, Brearley A, Danker K, Young K, Kopatz J, Hermann M, Berthemy A, Boyce S, Bentley J, Krauss R. Pharmacological SARM1 inhibition protects axon structure and function in paclitaxel-induced peripheral neuropathy. Brain. 2021 May 8:awab184. doi: 10.1093/brain/awab184. Epub ahead of print. PMID: 33964142), Hughes et al. (Hughes R O, Bosanac T, Mao X, Engber T M, DiAntonio A, Milbrandt J, Devraj R, Krauss R. Small Molecule SARM1 Inhibitors Recapitulate the SARM1−/− Phenotype and Allow Recovery of a Metastable Pool of Axons Fated to Degenerate. Cell Rep. 2021 Jan. 5; 34(1):108588. doi: 10.1016/j.celrep.2020.108588. PMID: 33406435; PMCID: PMC8179325), Sporny et al (Sporny M, Guez-Haddad J, Khazma T, Yaron A, Dessau M, Shkolnisky Y, Mim C, Isupov M N, Zalk R, Hons M, Opatowsky Y. Structural basis for SARM1 inhibition and activation under energetic stress. Elife. 2020 Nov. 13; 9:e62021. doi: 10.7554/eLife.62021. PMID: 33185189; PMCID: PMC7688312), WO 2018/057989 A1, WO 2020/081923 A1, and WO 2021/142006 A1. Certain dipeptidyl peptidase inhibitors (e.g., biphenyl or phenyl benzo imidazole derivatives) are disclosed in US 2005/0272765 A1. Certain benzyl benzoxazol derivatives as Met-kinase inhibitors are disclosed in WO 2008/148449 A1,

The present disclosure describes SARM1 inhibitors that can be used to prevent axonal degeneration in peripheral and central axonopathies and to provide a transformational disease-modifying treatment for these diseases or conditions.

### SUMMARY OF THE DISCLOSURE

One aspect of this disclosure provides a compound selected from compounds of the Formulae disclosed herein, a tautomer thereof, a solvate or stereoisomer of the compound or the tautomer, or a pharmaceutically acceptable salt of the foregoing, which can be employed in the treatment of various diseases or conditions, such as diseases or conditions caused by axonal degeneration. For example, disclosed herein is a compound of the following structural Formula I:

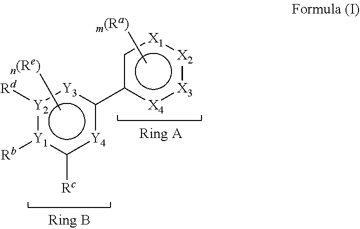

a tautomer thereof, a solvate or stereoisomer of the compound or the tautomer, or a pharmaceutically acceptable salt of the foregoing, wherein:
one or two of X₁, X₂, X₃, and X₄ in Ring A is N, and the rest of X₁, X₂, X₃, and X₄ are C;
Ring B is phenyl or one or two of Y₁, Y₂, Y₃, and Y₄ in Ring B is N, and the rest of Y₁, Y₂, Y₃, and Y₄ are C;
R^{a} is selected from H, —OR^{s}, halogen, —NR^{p}R^{q}, C₃-C₆ cycloalkyl, CN, and C₁-C₆ alkyl wherein the C₃-C₆ cycloalkyl and C₁-C₆ alkyl of R^{a} are optionally substituted with 1 to 3 groups selected from halogen, —OR^{s} and —NR^{p}R^{q},
R^{c} is selected from H, CN, —S(═O)_{w}NR^{p1}R^{q1}, —OR^{s}, halogen, —NR^{p1}R^{q1}, and C₁-C₆ alkyl optionally substituted with 1 to 3 groups selected from halogen, —OR^{s} and —NR^{p1}R^{q1}, provided that R^{c} is not CH₂OH;
R^{b} is absent or selected from H, halogen, —C(═O)(C₁-C₆ alkyl), —NR^{p1}R^{q1}, —OR^{s}, and C₁-C₆ alkyl optionally substituted with 1 to 3 groups selected from halogen, —OR^{s}, and —NR^{p1}R^{q1};
R^{d} is absent or selected from H, CN, —S(═O)_{w}NR^{p1}R^{q1}, —OR^{s}, halogen, —NR^{p1}R^{q1}, C₃-C₆ cycloalkyl and C₁-C₆ alkyl optionally substituted with 1 to 3 groups selected from halogen, —OR^{s} and —NR^{p1}R^{q1}, provided that R^{d} is not CH₂OH;
R^{b} and R^{e} may join together to form an optionally substituted 5- to 7-membered heterocyclic or heteroaromatic ring;
R^{b} and R^{d} may join together to form an optionally substituted 5- to 7-membered heterocyclic or heteroaromatic ring;
R^{e} is selected from H, halogen, —CN, C₁-C₆ alkyl optionally substituted with 1 to 3 groups selected from halogen, —OR^{s} and —NR^{p1}R^{q1};
R^{s}, for each occurrence, is independently selected from H, phenyl, 5-10 membered heteroaryl, C₃-C₆ cycloalkyl and C₁-C₆ alkyl, wherein the phenyl, 5-10 membered heteroaryl, C₃-C₆ cycloalkyl, and C₁-C₆ alkyl of R^{s} are each optionally substituted with 1-3 groups selected from halogen, —OH and —O(C₁-C₃ alkyl);
R^{p} and R^{q}, for each occurrence, are independently selected from hydrogen, phenyl, 9-10 membered aryl, 5-10 membered heteroaryl, C₃-C₆ cycloalkyl, and C₁-C₄ alkyl, wherein the phenyl, 5-10 membered heteroaryl, C₃-C₆ cycloalkyl, and C₁-C₄ alkyl of R^{p1} and R^{q1} are each optionally substituted with 1 to 3 groups selected from —COO(C₁-C₄ alkyl), halogen, —OH, —CN, —COOH, —CONH₂, —CONH(C₁-C₃ alkyl), —NH(C₁-C₃ alkyl), —O(C₁-C₃ alkyl), and C₁-C₄ alkyl;
R^{p1} and R^{q1}, for each occurrence, are independently selected from hydrogen, —CO(C₁-C₆ alkyl), —SO₂CH₃, C₃-C₆ cycloalkyl, and C₁-C₆ alkyl, wherein the C₃-C₆ cycloalkyl and C₁-C₆ alkyl of R^{p1} and R^{q1} are each optionally substituted with 1 to 3 halogens;
m is an integer independently selected from 0, 1, 2, and 3;
n is an integer independently selected from 0, 1, and 2; and
w is an integer selected from 1 and 2.

In one aspect of the disclosure, the compounds of the Formulae disclosed herein are selected from Compounds 1 to 120 shown below, a tautomer thereof, a solvate or stereoisomer of the compound or the tautomer, or a pharmaceutically acceptable salt of the foregoing.

In some embodiments, the disclosure provides pharmaceutical compositions comprising a compound of the Formulae disclosed herein, a tautomer thereof, a solvate or stereoisomer of the compound or the tautomer, or a pharmaceutically acceptable salt of the foregoing, and a pharmaceutically acceptable carrier. In some embodiments, the pharmaceutical compositions may comprise a compound selected from Compounds 1 to 120 shown below, a tautomer thereof, a solvate or stereoisomer of the compound or the tautomer, or a pharmaceutically acceptable salt of the foregoing, and a pharmaceutically acceptable carrier. These compositions may further comprise an additional active pharmaceutical agent.

Another aspect of the disclosure provides methods of treating a disease or condition, comprising administering to a subject in need thereof, a therapeutically effective amount of a compound of the Formulae disclosed herein, a tautomer thereof, a solvate or stereoisomer of the compound or the tautomer, or a pharmaceutically acceptable salt of the foregoing, or a pharmaceutical composition comprising any of the foregoing, wherein the disease or condition is selected amyotrophic lateral sclerosis (ALS), Parkinson's disease, Parkinsonian syndromes, ischemia, stroke, herpes infection, a demyelinating disease such as multiple sclerosis, traumatic brain injury, Sepsis, a chronic disease of PNS comprises inherited neuropathies, such as, but is not limited to Charcot-Marie-Tooth disease and chronic inflammatory demyelinating polyneuropathy (CIDP), an optic nerve disorder such as glaucoma, and retinal ganglion degeneration, colitis a metabolic disease or disorder such as diabetic neuropathy, nonalcoholic fatty liver disease (NAFLD) and nonalcoholic steatohepatitis (NASH) and a peripheral neuropathy like CIPN induced by various drugs.

A further aspect of the disclosure provides methods of treating a disease or condition caused by axonal degeneration, or neuronal damage mediated by SARM1 comprising administering to a subject in need thereof, a therapeutically effective amount of a compound of the Formulae disclosed herein, a tautomer thereof, a solvate or stereoisomer of the compound or the tautomer, or a pharmaceutically acceptable salt of the foregoing, or a pharmaceutical composition comprising any of the foregoing.

In some embodiments, the methods of treatment comprise administering to a subject in need thereof, a compound selected from Compounds 1 to 120 shown below, a tautomer thereof, a solvate or stereoisomer of the compound or the tautomer, or a pharmaceutically acceptable salt of the foregoing, or a pharmaceutical composition comprising any of the foregoing.

In some embodiments, the methods of treatment comprise administration of an additional active pharmaceutical agent to the subject in need thereof, either in the same pharmaceutical composition as a compound of the Formulae disclosed herein, a tautomer thereof, a solvate or stereoisomer of the compound or the tautomer, or a pharmaceutically acceptable salt of the foregoing, or in a separate composition. In some embodiments, the methods of treatment comprise administering a compound selected from Compounds 1 to 120 shown below, a tautomer thereof, a solvate or stereoisomer of the compound or the tautomer, or a pharmaceutically acceptable salt of the foregoing with an additional active pharmaceutical agent either in the same pharmaceutical composition or in a separate composition. When administered as a separate composition, the additional therapeutic agent may be administered prior to, at the same time as, or following administration of the compound, tautomer, solvate, stereoisomer, or a pharmaceutically acceptable salt disclosed herein.

Also disclosed herein are methods of modulating, e.g., inhibiting, SARM1 in a subject in need thereof, comprising contacting the subject with a compound of the Formulae disclosed herein, a tautomer thereof, a solvate or stereoisomer of the compound or the tautomer, or a pharmaceutically acceptable salt of the foregoing, or a pharmaceutical composition comprising any of the foregoing. In some embodiments, the methods of modulating, e.g., inhibiting, SARM1 in a subject in need thereof comprise contacting the subject with a compound selected from Compounds 1 to 120 shown below, a tautomer thereof, a solvate or stereoisomer of the compound or the tautomer, or a pharmaceutically acceptable salt of the foregoing, or a pharmaceutical composition comprising any of the foregoing.

Also disclosed herein are methods of inhibiting or preventing axonal degeneration in a subject in need thereof, comprising contacting the subject with a compound of the Formulae disclosed herein, a tautomer thereof, a solvate or stereoisomer of the compound or the tautomer, or a pharmaceutically acceptable salt of the foregoing, or a pharmaceutical composition comprising any of the foregoing. In some embodiments, the methods of inhibiting or preventing axonal degeneration or neuronal damage mediated by SARM1 in a subject in need thereof comprise contacting the subject with a compound selected from Compounds 1 to 120 shown below, a tautomer thereof, a solvate or stereoisomer of the compound or the tautomer, or a pharmaceutically acceptable salt of the foregoing, or a pharmaceutical composition comprising any of the foregoing.

### DETAILED DESCRIPTION OF THE DISCLOSURE

### I. Definitions

The term “a” or “an” when referring to a noun as used herein encompasses the expression “at least one” and therefore encompasses both singular and plural units of the noun. For example, “an additional pharmaceutical agent” means a single or two or more additional pharmaceutical agents.

The term “alkyl” refers to a hydrocarbon group selected from linear and branched saturated hydrocarbon groups, containing 1-20, e.g., 1-18, 1-12, 1-10, 1-8, 1-6, 1-4, or 1-3, carbon atoms. Examples of the alkyl group include methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), and 1,1-dimethylethyl or t-butyl (“t-Bu”). Other examples of an alkyl group include 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, and 3,3-dimethyl-2-butyl groups. Lower alkyl contains 1-8, preferably 1-6, more preferably 1-4 carbon atoms, and more preferably 1-3 carbon atoms.

The term “alkenyl” refers to a hydrocarbon group selected from linear and branched hydrocarbon groups, comprising at least one C═C double bond and 2-20, e.g., 2-18, 2-12, 2-10, 2-8, 2-6, or 2-4, carbon atoms. Examples of the alkenyl group may be selected from ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl, and hexa-1,3-dienyl groups. Lower alkenyl contains 2-8, preferably 2-6, and more preferably 2-4 carbon atoms.

The term “alkynyl” refers to a hydrocarbon group selected from linear and branched hydrocarbon groups, comprising at least one C≡C triple bond and 2-20, e.g., 2-18, 2-12, 2-10, 2-8, 2-6, or 2-4, carbon atoms. Examples of the alkynyl group include ethynyl, 1-propynyl, 2-propynyl (propargyl), 1-butynyl, 2-butynyl, and 3-butynyl groups. Lower alkynyl contains 2-8, preferably 2-6, and more preferably 2-4 carbon atoms.

The term “heteroalkyl” refers to an alkyl group, as defined herein, in which one or more of the constituent carbon atoms have been replaced by a heteroatom, e.g., nitrogen, oxygen, or sulfur, e.g., CH₃CH₂OH, CH₃CH₂OC₂H₅, CH₃CH₂SH, CH₃CH₂SC₂H₅, CH₃CH₂NH₂, CH₃CH₂NHC₂H₅, etc. In some embodiments, in addition to the replacement of one or more of the constituent carbon atoms by nitrogen, oxygen, or sulfur, a heteroalkyl group is further optionally substituted as defined herein.

The term “cycloalkyl” refers to a hydrocarbon group selected from saturated and partially unsaturated cyclic hydrocarbon groups, e.g., monocyclic and polycyclic (e.g., bicyclic and tricyclic) groups. For example, the cycloalkyl group may be of 3-12, 3 to 10, or 3-8, or 3-6, or 3-4, or 5-6 carbon atoms. Even further for example, the cycloalkyl group may be a monocyclic group of 3-12, or 3-8, or 3-6, or 3-4, or 5-6 carbon atoms. Examples of the monocyclic cycloalkyl group include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and cyclododecyl groups. Examples of the bicyclic cycloalkyl groups include those having 7-12 ring atoms arranged as a bicycle ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. The ring may be saturated or have at least one double bond (i.e., partially unsaturated), but is not fully conjugated, and is not aromatic, as aromatic is defined herein.

The term “heterocyclic” or “heterocycle” or “heterocyclyl” refers to a ring selected from 4- to 12-membered, e.g., 3- to 6-membered, 3- to 5-membered, 4- to 5-membered, or 5- to 6-membered, monocyclic, bicyclic, and tricyclic, saturated and partially unsaturated rings comprising at least one carbon atom in addition to 1, 2, 3, or 4 heteroatoms, selected from, e.g., oxygen, sulfur, nitrogen, and silicon. “Heterocycle” also refers to a 5- to 7-membered heterocyclic ring comprising at least one heteroatom selected from N, O, and S fused with 5-, 6-, and/or 7-membered cycloalkyl, carbocyclic aromatic, or heteroaromatic ring, provided that the point of attachment is at the heterocyclic ring when the heterocyclic ring is fused with a carbocyclic aromatic or a heteroaromatic ring, and that the point of attachment can be at the cycloalkyl or heterocyclic ring when the heterocyclic ring is fused with cycloalkyl.

“Heterocycle” also refers to an aliphatic spirocyclic ring comprising at least one heteroatom selected from N, O, and S, provided that the point of attachment is at the heterocyclic ring. The rings may be saturated or have at least one double bond (i.e., partially unsaturated). A heterocycle may be substituted with oxo. The point of the attachment may be carbon or heteroatom in the heterocyclic ring. A heterocycle is not a heteroaryl as defined herein.

Examples of heterocycles include, but are not limited to, (as numbered from the linkage position assigned priority 1) 1-pyrrolidinyl, 2-pyrrolidinyl, 2,4-imidazolidinyl, 2,3-pyrazolidinyl, 1-piperidinyl, 2-piperidinyl, 3-piperidinyl, 4-piperidinyl, 2,5-piperazinyl, pyranyl, 2-morpholinyl, 3-morpholinyl, oxiranyl, aziridinyl, thiiranyl, azetidinyl, oxetanyl, thietanyl, 1,2-dithietanyl, 1,3-dithietanyl, dihydropyridinyl, tetrahydropyridinyl, thiomorpholinyl, thioxanyl, piperazinyl, homopiperazinyl, homopiperidinyl, azepanyl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl, 1,4-diazepanyl, 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinylimidazolinyl, pyrimidinonyl, 1,1-dioxo-thiomorpholinyl, 3-azabicyco[3.1.0]hexanyl, 3-azabicyclo[4.1.0]heptanyl and azabicyclo[2.2.2]hexanyl. Substituted heterocycle also includes ring systems substituted with one or more oxo moieties, such as piperidinyl N-oxide, morpholinyl-N-oxide, 1-oxo-1-thiomorpholinyl, and 1, 1-dioxo-1-thiomorpholinyl.

The term “fused ring” herein refers to a polycyclic ring system, e.g., a bicyclic or tricyclic ring system, in which two rings share only two ring atoms and one bond in common. Examples of fused rings may comprise a fused bicyclic cycloalkyl ring such as those having from 7 to 12 ring atoms arranged as a bicyclic ring selected from [4,4], [4,5], [5,5], [5,6] and [6,6] ring systems as mentioned above; a fused bicyclic aryl ring such as 7 to 12 membered bicyclic aryl ring systems as mentioned above, a fused tricyclic aryl ring such as 10 to 15 membered tricyclic aryl ring systems mentioned above; a fused bicyclic heteroaryl ring such as 8- to 12-membered bicyclic heteroaryl rings as mentioned above, a fused tricyclic heteroaryl ring such as 11- to 14-membered tricyclic heteroaryl rings as mentioned above; and a fused bicyclic or tricyclic heterocyclyl ring as mentioned above.

The term “heteroatom” means one or more of oxygen, sulfur, nitrogen, phosphorus, and silicon, including, any oxidized form of nitrogen or sulfur; the quaternized form of any basic nitrogen or a substitutable nitrogen of a heterocyclic ring, for example N (as in 3,4-dihydro-2H-pyrrolyl), NH (as in pyrrolidinyl) or NR⁺ (wherein R is, e.g., an optionally substituted alkyl group) (as in N-substituted pyrrolidinyl).

The term “unsaturated”, as used herein, means that a moiety has one or more units or degrees of unsaturation. Unsaturation is the state in which not all of the available valence bonds in a compound are satisfied by substituents and thus the compound contains one or more double or triple bonds.

The term “alkoxy” as used herein, refers to an alkyl group, as defined above, wherein one carbon of the alkyl group is replaced by an oxygen atom, provided that the oxygen atom is linked between two carbon atoms.

The term “halogen” includes F, Cl, Br, and I, i.e., fluoro, chloro, bromo, and iodo, respectively.

As used herein, a “CN,” “cyano” or “nitrile” group refers to —C≡N.

As used herein, an “aromatic ring” refers to a carbocyclic or heterocyclic ring that contains conjugated, planar ring systems with delocalized pi electron orbitals comprised of [4n+2]p orbital electrons, wherein n is an integer of 0 to 6. A “non-aromatic” ring refers to a carbocyclic or heterocyclic that does not meet the requirements set forth above for an aromatic ring, and can be either completely or partially saturated. Non-limiting examples of aromatic rings include aryl and heteroaryl rings that are further defined as follows. An “aromatic ring” may be depicted as a cycle with conjugated double bonds, such as

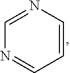

or as a cycle with an inside circle such as

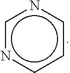

The term “aryl” herein refers to a group selected from: monocyclic carbocyclic aromatic rings, for example, phenyl; bicyclic ring systems such as 7-12 membered, e.g., 9-10 membered, bicyclic ring systems wherein at least one ring is carbocyclic and aromatic, selected, for example, from naphthalene, indane, and 1,2,3,4-tetrahydroquinoline; and tricyclic ring systems such as 10-15 membered tricyclic ring systems wherein at least one ring is carbocyclic and aromatic, for example, fluorene.

For example, the aryl group may be a 6-membered carbocyclic aromatic ring fused to a 5- to 7-membered cycloalkyl or heterocyclic ring optionally comprising at least one heteroatom selected from N, O, and S, provided that the point of attachment is at the carbocyclic aromatic ring when the carbocyclic aromatic ring is fused with a heterocyclic ring, and the point of attachment can be at the carbocyclic aromatic ring or at the cycloalkyl group when the carbocyclic aromatic ring is fused with a cycloalkyl group. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene.

The term “heteroaryl” refers to a group selected from: 5- to 7-membered, e.g., 5- to 6-membered, aromatic, monocyclic rings comprising 1, 2, 3, or 4 heteroatoms selected from N, O, and S, with the remaining ring atoms being carbon; 8- to 12-membered bicyclic rings comprising 1, 2, 3, or 4 heteroatoms, selected from N, O, and S, with the remaining ring atoms being carbon and wherein at least one ring is aromatic and at least one heteroatom is present in the aromatic ring; and 11- to 14-membered tricyclic rings comprising 1, 2, 3, or 4 heteroatoms, selected from N, O, and S, with the remaining ring atoms being carbon and wherein at least one ring is aromatic and at least one heteroatom is present in an aromatic ring.

For example, the heteroaryl group may be a 5- to 7-membered heterocyclic aromatic ring fused to a 5- to 7-membered cycloalkyl ring. For such fused, bicyclic heteroaryl ring systems wherein only one of the rings comprises at least one heteroatom, the point of attachment may be at the heteroaromatic ring or at the cycloalkyl ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1.

Examples of the heteroaryl group include, but are not limited to, (as numbered from the linkage position assigned priority 1) pyridyl (such as 2-pyridyl, 3-pyridyl, or 4-pyridyl), cinnolinyl, pyrazinyl, 2,4-pyrimidinyl, 3,5-pyrimidinyl, 2,4-imidazolyl, imidazopyridinyl, isoxazolyl, oxazolyl, thiazolyl, isothiazolyl, thiadiazolyl, tetrazolyl, thienyl, triazinyl, benzothienyl, furyl, benzofuryl, benzoimidazolyl, indolyl, isoindolyl, indolinyl, phthalazinyl, pyrazinyl, pyridazinyl, pyrrolyl, triazolyl, quinolinyl, isoquinolinyl, pyrazolyl, pyrrolopyridinyl (such as 1H-pyrrolo[2,3-b]pyridin-5-yl), pyrazolopyridinyl (such as 1H-pyrazolo[3,4-b]pyridin-5-yl), benzoxazolyl (such as benzo[d]oxazol-6-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl, benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), indazolyl (such as 1H-indazol-5-yl) and 5,6,7,8-tetrahydroisoquinolinyl.

The term “acyl” refers to a substituent group where a point of attachment in the substituent group is a carbonyl. Exemplary acyl groups include, but are not limited to, —C(═O)R′, —C(═O)NR′R″, or —C(═O)OR′, wherein R′ and R″ are independently selected from hydrogen, alkyl, alkenyl, alkynyl, heteroalkyl, heteroalkenyl, heteroalkynyl, cycloalkyl, aryl, heterocyclyl, or heteroaryl, any of which may be further substituted by one or more substituents.

Some of the compounds may exist with different points of attachment of hydrogen, referred to as “tautomers.” For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable.

The compounds, tautomers, solvates, or pharmaceutically acceptable salts of the disclosure may contain an asymmetric center and may thus exist as enantiomers. For example, where the compounds possess two or more asymmetric centers, they may additionally exist as diastereoisomers. Enantiomers and diastereoisomers fall within the broader class of stereoisomers. All such possible stereoisomers as substantially pure resolved enantiomers, racemic mixtures thereof, as well as mixtures of diastereoisomers are intended to be included in this disclosure. All stereoisomers of the compounds, tautomers, solvates, and pharmaceutically acceptable salts thereof are intended to be included. Unless specifically mentioned otherwise, reference to one isomer applies to any of the possible isomers. Whenever the isomeric composition is unspecified, all possible isomers are included.

Diastereomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereoisomers using optically active resolving agents. Racemic mixtures of chiral compounds of the disclosure can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereoisomers, and conversion to the pure stereoisomers, and (3) separation of the substantially pure or enriched stereoisomers directly under chiral conditions.

The term “substantially pure” in the context of stereoisomers means that the target stereoisomer contains no more than 35%, such as no more than 30%, further such as no more than 25%, even further such as no more than 20%, by weight of any other stereoisomer(s). In some embodiments, the term “substantially pure” means that the target stereoisomer contains no more than 10%, for example, no more than 5%, such as no more than 1%, by weight of any other stereoisomer(s).

Unless otherwise indicated, structures depicted herein are meant to include all isomeric forms of the structure, e.g., racemic mixtures, cis/trans isomers, geometric (or conformational) isomers, such as (Z) and (E) double bond isomers, and (Z) and (E) conformational isomers. Therefore, geometric and conformational mixtures of the compounds disclosed herein are within the scope of the disclosure. Unless otherwise stated, all tautomeric forms of the compounds of the disclosure are within the scope of the disclosure.

The disclosure provides pharmaceutically acceptable salts of the disclosed compounds, tautomers, solvates, and stereoisomers. A salt of a compound is formed between an acid and a basic group of the compound, such as an amino functional group, or a base and an acidic group of the compound, such as a carboxyl functional group.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this disclosure.

“Pharmaceutically acceptable salts” include, but are not limited to salts with inorganic acids, selected, for example, from hydrochlorates, phosphates, diphosphates, hydrobromates, sulfates, sulfinates, and nitrates; as well as salts with organic acids, selected, for example, from malates, maleates, fumarates, tartrates, succinates, citrates, lactates, methanesulfonates, p-toluenesulfonates, 2-hydroxyethylsulfonates, benzoates, salicylates, stearates, alkanoates such as acetate, and salts with HOOC—(CH₂)n-COOH, wherein n is selected from 0 to 4. Similarly, examples of pharmaceutically acceptable cations include, but are not limited to, sodium, potassium, calcium, magnesium, aluminum, lithium, and ammonium. Suitable pharmaceutically acceptable salts are, for example, those disclosed in S. M. Berge, et al. J. Pharmaceutical Sciences, 1977, 66, pp. 1 to 19.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, benzenesulfonic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid, and acetic acid. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate (i.e., caprate), caprylate, acrylate, formate, isobutyrate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, tartrate, methanesulfonate, propanesulfonate, naphthalene-1-sulfonate, naphthalene-2-sulfonate, mandelate and other salts. In some embodiments, pharmaceutically acceptable acid addition salts include those formed with mineral acids such as hydrochloric acid and hydrobromic acid, and those formed with organic acids such as maleic acid.

Pharmaceutically acceptable salts derived from appropriate bases include alkali metal, alkaline earth metal, ammonium, and N+(C₁₄ alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium salts. Further non-limiting examples of pharmaceutically acceptable salts include salts of ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

If a compound is obtained as an acid addition salt, the free base can be obtained by basifying a solution of the acid addition salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

The compounds, tautomers, solvates, stereoisomers, and pharmaceutically acceptable salts of the disclosure may also contain unnatural proportions of atomic isotopes at one or more of the atoms that constitute such compounds. For example, —CD₃, —CD₂H or —CDH₂ contains one or more deuteriums in place of hydrogen. For example, the compounds may be radiolabeled with radioactive isotopes, such as for example tritium (³H), iodine-125 (¹²⁵I) or carbon-14 (¹⁴C). All isotopic variations of the compounds of the disclosure, whether radioactive or not, are intended to be encompassed within the scope of the disclosure.

As used herein, “optionally substituted” is interchangeable with the phrase “substituted or unsubstituted.” In general, the term “substituted,” refers to the replacement of a hydrogen radical in a given structure with the radical of a specified substituent. Unless otherwise indicated, an “optionally substituted” group may have a substituent at each substitutable position of the group, and when more than one position in any given structure may be substituted with more than one substituent chosen from a specified group, the substituent may be either the same or different at every position. Combinations of substituents envisioned by this disclosure are those that result in the formation of stable or chemically feasible compounds.

In some embodiments, substituents are independently selected from optionally substituted heteroatom and optionally substituted, optionally hetero-, optionally cyclic C₁-C₁₈ hydrocarbyl, particularly wherein the optionally substituted, optionally hetero-, optionally cyclic C₁-C₁₈ hydrocarbyl is optionally-substituted, optionally hetero-, optionally cyclic alkyl, alkenyl or alkynyl, or optionally-substituted, optionally hetero-, aryl; and/or the optionally substituted heteroatom is halogen, optionally substituted hydroxyl (such as alkoxy, aryloxy), optionally substituted acyl (such as formyl, alkanoyl, carbamoyl, carboxyl, amido), optionally substituted amino (such as amino, alkylamino, dialkylamino, amido, sulfamidyl), optionally substituted thiol (such as mercapto, alkylthiol, aryl thiol), optionally substituted sulfinyl or sulfonyl (such as alkylsulfinyl, arylsulfinyl, alkyl sulfonyl, arylsulfonyl), nitro, or cyano.

In some embodiments, substituents are independently selected from: halogen, —R′, —OR′, ═O, =NR′, =N—OR′, —NR′R″, —SR′, —SiR′R″R′, —OC(═O)R′, —C(═O)R′, —CO₂R′, —C(═O)NR′R″, —OC(═O)NR′R″, —NR″C(═O)R′, —NR′—C(═O)NR″R′″, —NR′—SO₂NR″R″′, —NR″CO₂R′, —NH—C(NH₂)=NH, —NR′C(NH₂)=NH, —NH—C(NH₂)=NR′, —S(O)R′, —SO₂R′, —SO₂NR′R″, —NR″SO₂R, —CN, —NO₂, —N₃, —CH(Ph)₂, perfluoro(C₁-C₄)alkoxy, and perfluoro(C₁-C₄)alkyl, in a number ranging from zero to three, with those groups having zero, one, or two substituents being particularly preferred. R′, R″ and R″′ each independently refer to hydrogen, unsubstituted C₁-C₅ alkyl and heteroalkyl, C₁-C₅ alkyl and heteroalkyl substituted with one to three halogens, unsubstituted aryl, aryl substituted with one to three halogens, unsubstituted alkyl, alkoxy, or thioalkoxy groups, or aryl-(C₁-C₄) alkyl groups. When R′ and R″ are attached to the same nitrogen atom, they can be combined with the nitrogen atom to form a 5-, 6- or 7-membered ring. Hence, —NR′R″ includes 1-pyrrolidinyl and 4-morpholinyl. When the aryl group is 1,2,3,4-tetrahydronaphthalenyl, it may be substituted with a substituted or unsubstituted C₃-C₇ spirocycloalkyl group. The C₃-C₇ spirocycloalkyl group may be substituted in the same manner as defined herein for “cycloalkyl.”

In some embodiments, substituents are selected from: halogen, —R′, —OR′, ═O, —NR′R″, —SR′, —SiR′R″R″′, —OC(═O)R′, —C(═O)R′, —CO₂R′, —C(═O)NR′R″, —OC(═O)NR′R″, —NR″C(═O)R′, —NR″CO₂R′, —NR′—SO₂NR″R″′, —S(═O)R′, —SO₂R′, —SO₂NR′R″, —NR″SO₂R, —CN, —NO₂, perfluoro C₁-C₄ alkoxy and perfluoro C₁-C₄ alkyl, where R′ and R″ are as defined above.

In some embodiments, substituents are independently selected from substituted or unsubstituted heteroatom, substituted or unsubstituted, 0-3 heteroatom-containing C₁-C₆ alkyl (e.g., C₁-C₃ alkyl or C₁-C₂ alkyl), substituted or unsubstituted, 0-3 heteroatom-containing C₂-C₆ alkenyl (e.g., C₂-C₄ alkenyl), substituted or unsubstituted, 0-3 heteroatom-containing C₂-C₆ alkynyl (e.g., C₂-C₄ alkynyl), or substituted or unsubstituted, 0-3 heteroatom-containing C₆-C₁₄ aryl (e.g., C₅-C₆ aryl), wherein each heteroatom is independently oxygen, phosphorus, sulfur, or nitrogen.

In some embodiments, substituents are independently selected from aldehyde, aldimine, alkanoyloxy, alkoxy, alkoxycarbonyl, alkyloxy, alkyl, alkenyl, alkynyl, amine, azo, halogen, carbamoyl, carbonyl, carboxamido, carboxyl, cyanyl, ester, haloformyl, hydroperoxyl, hydroxyl, imine, isocyanide, iscyante, N-tert-butoxycarbonyl, nitrate, nitrile, nitrite, nitro, nitroso, phosphate, phosphono, sulfide, sulfonyl, sulfo, sulfhydryl, thiol, thiocyanyl, trifluoromethyl, and trifluromethyl ether (OCF₃) groups.

Preferred substituents are disclosed herein and exemplified in the tables, structures, examples, and claims, and may be applied across different compounds of this disclosure. For example, substituents of a given compound may be combinatorically used with other compounds.

It may be advantageous to separate reaction products from one another and/or from starting materials. The desired products of each step or series of steps are separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example, reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art may apply such techniques to achieve a desired separation.

Non-limiting examples of suitable solvents that may be used in this disclosure include water, methanol (MeOH), ethanol (EtOH), dichloromethane or methylene chloride (CH₂Cl₂), toluene, acetonitrile (MeCN), dimethylformamide (DMF), dimethyl sulfoxide (DMSO), methyl acetate (MeOAc), ethyl acetate (EtOAc), heptanes, isopropyl acetate (IPAc), tert-butyl acetate (t-BuOAc), isopropyl alcohol (IPA), tetrahydrofuran (THF), 2-methyl tetrahydrofuran (2-Me THF), methyl ethyl ketone (MEK), tert-butanol, diethyl ether (Et₂O), methyl-tert-butyl ether (MTBE), 1,4-dioxane, and N-methyl pyrrolidone (NMP).

Non-limiting examples of suitable bases that may be used in this disclosure include 1,8-diazabicyclo[5.4.0]undec-7-ene (DBU), potassium tert-butoxide (KOtBu), potassium carbonate (K₂CO₃), N-methylmorpholine (NMM), triethylamine (Et₃N; TEA), diisopropyl-ethyl amine (i-Pr₂EtN; DIPEA), pyridine, potassium hydroxide (KOH), sodium hydroxide (NaOH), lithium hydroxide (LiOH), and sodium methoxide (NaOMe; NaOCH₃).

The term “subject” refers to an animal including a human.

The term “therapeutically effective amount” refers to the amount of a compound that produces a desired effect for which it is administered (e.g., improvement in a disease or condition, lessening the severity of a disease or condition, and/or reducing progression of a disease or condition, e.g., ALS, Parkinson's disease, multiple sclerosis, traumatic brain injury, diabetic neuropathy, and CIPN. The disease or condition may be caused by axonal degeneration. The exact amount of a therapeutically effective amount will depend on the purpose of the treatment and will be ascertainable by one skilled in the art using known techniques (see, e.g., Lloyd (1999), The Art, Science and Technology of Pharmaceutical Compounding).

As used herein, the term “treatment” and its cognates refer to slowing or stopping disease progression. “Treatment” and its cognates as used herein include, but are not limited to the following: complete or partial remission, curing a disease or condition or a symptom thereof, lower risk of a disease or condition, e.g., ALS, Parkinson's disease, multiple sclerosis, traumatic brain injury, diabetic neuropathy, and CIPN. The disease or condition may be caused by axonal degeneration. Improvements in or lessening the severity of any of these symptoms can be assessed according to methods and techniques known in the art.

The terms “about” and “approximately,” when used in connection with a number such as a percentage include the number as specified, and a range of the number (e.g., a range of percentages, for example, a range of +10% with respect to a specific point value) that is recognized by one of ordinary skill in the art.

### II. Compounds and Compositions

In a first embodiment, a compound of this disclosure is a compound of the following structural Formula I:

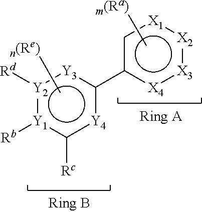

a tautomer thereof, a solvate or stereoisomer of the compound or the tautomer, or a pharmaceutically acceptable salt of the foregoing, wherein:
one or two of X₁, X₂, X₃, and X₄ in Ring A is N, and the rest of X₁, X₂, X₃, and X₄ are C;
Ring B is phenyl or one or two of Y₁, Y₂, Y₃, and Y₄ in Ring B is N, and the rest of Y₁, Y₂, Y₃, and Y₄ are C;
R^{a} is selected from H, —OR^{s}, halogen, —NR^{p}R^{q}, C₃-C₆ cycloalkyl, CN, and C₁-C₆ alkyl wherein the C₃-C₆ cycloalkyl and C₁-C₆ alkyl of R^{a} are optionally substituted with 1 to 3 groups selected from halogen, —OR^{s} and —NR^{p}R^{q},
R^{c} is selected from H, CN, —S(═O)_{w}NR^{p1}R^{q1}, —OR^{s}, halogen, —NR^{p1}R^{q1}, and C₁-C₆ alkyl optionally substituted with 1 to 3 groups selected from halogen, —OR^{s} and —NR^{p1}R^{q1}, provided that R^{c} is not CH₂OH;
R^{b} is absent or selected from H, halogen, —C(═O)(C₁-C₆ alkyl), —NR^{p1}R^{q1}, —OR^{s}, and C₁-C₆ alkyl optionally substituted with 1 to 3 groups selected from halogen, —OR^{s}, and —NR^{p1}R^{q1};
R^{d} is absent or selected from H, CN, —S(═O)_{w}NR^{p1}R^{q1}, —OR^{s}, halogen, —NR^{p1}R^{q1}, C₃-C₆ cycloalkyl and C₁-C₆ alkyl optionally substituted with 1 to 3 groups selected from halogen, —OR^{s} and —NR^{p1}R^{q1}, provided that R^{d} is not CH₂OH;
R^{b} and R^{e} may join together to form an optionally substituted 5- to 7-membered heterocyclic or heteroaromatic ring;
R^{b} and R^{d} may join together to form an optionally substituted 5- to 7-membered heterocyclic or heteroaromatic ring;
R^{c} is selected from H, halogen, —CN, C₁-C₆ alkyl optionally substituted with 1 to 3 groups selected from halogen, —OR^{s} and —NR^{p1}R^{q1};
R^{s}, for each occurrence, is independently selected from H, phenyl, 5-10 membered heteroaryl, C₃-C₆ cycloalkyl and C₁-C₆ alkyl, wherein the phenyl, 5-10 membered heteroaryl, C₃-C₆ cycloalkyl, and C₁-C₆ alkyl of R^{s} are each optionally substituted with 1-3 groups selected from halogen, —OH and —O(C₁-C₃ alkyl);
R^{p1} and R^{q1}, for each occurrence, are independently selected from hydrogen, phenyl, 9-10 membered aryl, 5-10 membered heteroaryl, C₃-C₆ cycloalkyl, and C₁-C₄ alkyl, wherein the phenyl, 9-10 membered aryl, 5-10 membered heteroaryl, C₃-C₆ cycloalkyl, and C₁-C₄ alkyl of R^{p} and R^{q} are each optionally substituted with 1 to 3 groups selected from —COO(C₁-C₄ alkyl), halogen, —OH, —CN, —COOH, —CONH₂, —CONH(C₁-C₃ alkyl), —NH(C₁-C₃ alkyl), —O(C₁-C₃ alkyl), and C₁-C₄ alkyl;
R^{p1} and R^{q1}, for each occurrence, are independently selected from hydrogen, —CO(C₁-C₆ alkyl), —SO₂CH₃, C₃-C₆ cycloalkyl, and C₁-C₆ alkyl, wherein the C₃-C₆ cycloalkyl and C₁-C₆ alkyl of R^{p1} and R^{q1} are each optionally substituted with 1 to 3 halogens;
m is an integer independently selected from 0, 1, 2, and 3;
n is an integer independently selected from 0, 1, and 2; and
w is an integer selected from 1 and 2.

Combinations of substituents as disclosed herein are those that result in the formation of stable or chemically feasible compounds. For abbreviation or according to common practice, certain hydrogen atoms attached to a certain atom (e.g., a carbon atom C or a nitrogen atom N) are not specifically spelled out in a chemical structure, formula, or notation; hydrogen atoms are deemed to be present to the extent the valences of the certain atom (e.g., C or N) are completed.

In a second embodiment, in a compound, tautomer, a solvate or stereoisomer of the compound or the tautomer, or pharmaceutically acceptable salt of this disclosure, Ring A is a pyridinyl, pyrimidinyl, or pyridazinyl group; all other variables not specifically defined herein are as defined in any one of the preceding embodiments.

In a third embodiment, in a compound, tautomer, a solvate or stereoisomer of the compound or the tautomer, or pharmaceutically acceptable salt of this disclosure, Ring B is a phenyl, or a pyridinyl group wherein one of Y₁, Y₂, Y₃, and Y₄ is N, and the rest of Y₁, Y₂, Y₃, and Y₄ are C; all other variables not specifically defined herein are as defined in any one of the preceding embodiments.

In a fourth embodiment, a compound of the disclosure is a compound of the following structural Formula IIa:

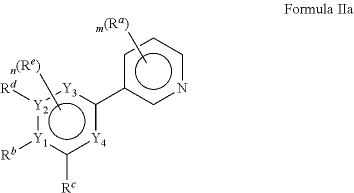

a tautomer thereof, a solvate or stereoisomer of the compound or the tautomer, or a pharmaceutically acceptable salt of the foregoing; and all other variables not specifically defined herein are as defined in any one of the preceding embodiments.

In a fifth embodiment, a compound of the disclosure is a compound of the following structural Formula IIb:

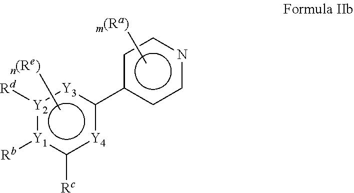

a tautomer thereof, a solvate or stereoisomer of the compound or the tautomer, or a pharmaceutically acceptable salt of the foregoing; and all other variables not specifically defined herein are as defined in any one of the preceding embodiments.

In a sixth embodiment, a compound of the disclosure is a compound of the following structural Formula IIc:

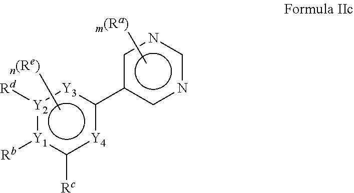

a tautomer thereof, a solvate or stereoisomer of the compound or the tautomer, or a pharmaceutically acceptable salt of the foregoing; and all other variables not specifically defined herein are as defined in any one of the preceding embodiments.

In a seventh embodiment, a compound of the disclosure is a compound of the following structural Formula IId:

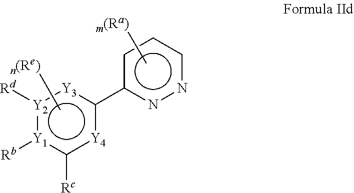

a tautomer thereof, a solvate or stereoisomer of the compound or the tautomer, or a pharmaceutically acceptable salt of the foregoing; and all other variables not specifically defined herein are as defined in any one of the preceding embodiments.

In an eighth embodiment, a compound of the disclosure is a compound of the following structural Formula IIIa:

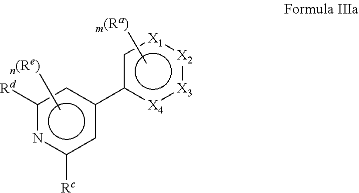

a tautomer thereof, a solvate or stereoisomer of the compound or the tautomer, or a pharmaceutically acceptable salt of the foregoing; and all other variables not specifically defined herein are as defined in any one of the preceding embodiments.

In a ninth embodiment, a compound of the disclosure is a compound of the following structural Formula IIIb:

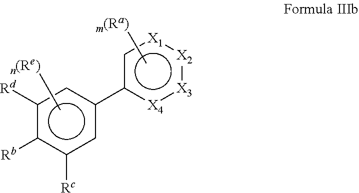

a tautomer thereof, a solvate or stereoisomer of the compound or the tautomer, or a pharmaceutically acceptable salt of the foregoing; and all other variables not specifically defined herein are as defined in any one of the preceding embodiments.

In a tenth embodiment, a compound of the disclosure is a compound of the following structural Formula IIIc:

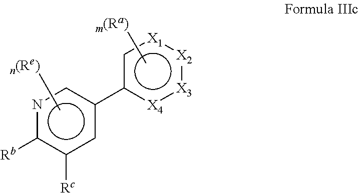

a tautomer thereof, a solvate or stereoisomer of the compound or the tautomer, or a pharmaceutically acceptable salt of the foregoing; and all other variables not specifically defined herein are as defined in any one of the preceding embodiments.

In an eleventh embodiment, a compound of the disclosure is a compound of the following structural Formula IIId:

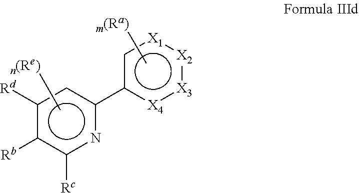

a tautomer thereof, a solvate or stereoisomer of the compound or the tautomer, or a pharmaceutically acceptable salt of the foregoing; and all other variables not specifically defined herein are as defined in any one of the preceding embodiments.

In a twelfth embodiment, a compound of the disclosure is a compound of the following structural Formula IIIe:

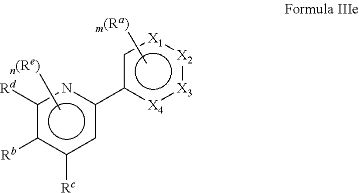

a tautomer thereof, a solvate or stereoisomer of the compound or the tautomer, or a pharmaceutically acceptable salt of the foregoing; and all other variables not specifically defined herein are as defined in any one of the preceding embodiments.

In a thirteenth embodiment, a compound of the disclosure is a compound of the following structural Formula IIIf:

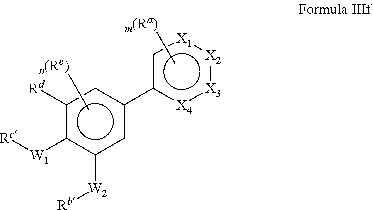

a tautomer thereof, a solvate or stereoisomer of the compound or the tautomer, or a pharmaceutically acceptable salt of the foregoing;
wherein W₁ and W₂ are each independently selected from NH and O; and wherein
when W₁ is O, R^{c′} is selected from H, C₃-C₆ cycloalkyl, and C₁-C₆ alkyl, wherein the C₃-C₆ cycloalkyl and C₁-C₆ alkyl of R^{c′} are each optionally substituted with 1-3 groups selected from halogen, —OH and —O(C₁-C₃alkyl);
when W₁ is NH, R^{c′} is selected from hydrogen, —CO(C₁-C₆ alkyl), —SO₂CH₃, C₃-C₆ cycloalkyl, and C₁-C₆ alkyl, wherein the C₃-C₆ cycloalkyl and C₁-C₆ alkyl of R^{c′} are each optionally substituted with 1 to 3 halogens;
when W₂ is O, R^{b′} is selected from H, C₃-C₆ cycloalkyl and C₁-C₆ alkyl, wherein the C₃-C₆ cycloalkyl and C₁-C₆ alkyl of R^{s} are each optionally substituted with 1-3 groups selected from halogen, —OH and —O(C₁-C₃ alkyl);
when W₂ is NH, R^{b} is selected from hydrogen, —CO(C₁-C₆ alkyl, —SO₂CH₃, C₃-C₆ cycloalkyl, and C₁-C₆ alkyl, wherein the C₃-C₆ cycloalkyl and C₁-C₆ alkyl of R^{b} are each optionally substituted with 1 to 3 halogens; and all other variables not specifically defined herein are as defined in any one of the preceding embodiments.

In a fourteenth embodiment, a compound of the disclosure is a compound of the following structural Formula IVa:

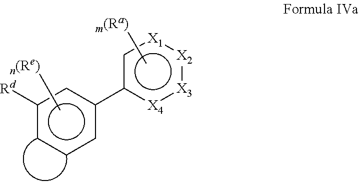

a tautomer thereof, a solvate or stereoisomer of the compound or the tautomer, or a pharmaceutically acceptable salt of the foregoing, wherein R^{b} and R^{c} join together to form an optionally substituted 5- to 7-membered heterocyclic or heteroaromatic ring; and all other variables not specifically defined herein are as defined in any one of the preceding embodiments.

In a fifteenth embodiment, a compound of the disclosure is a compound of the following structural Formula IVb:

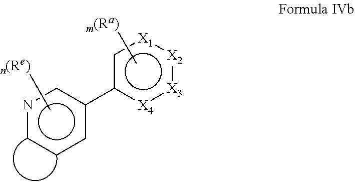

a tautomer thereof, a solvate or stereoisomer of the compound or the tautomer, or a pharmaceutically acceptable salt of the foregoing, wherein R^{b} and R^{c} join together to form an optionally substituted 5- to 7-membered heterocyclic or heteroaromatic ring; and all other variables not specifically defined herein are as defined in any one of the preceding embodiments.

In a sixteenth embodiment, a compound of the disclosure is a compound of the following structural Formula IVc:

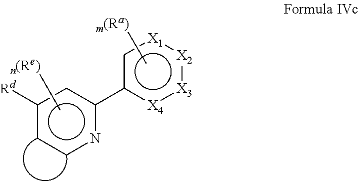

a tautomer thereof, a solvate or stereoisomer of the compound or the tautomer, or a pharmaceutically acceptable salt of the foregoing, wherein R^{b} and R^{c} join together to form an optionally substituted 5- to 7-membered heterocyclic or heteroaromatic ring; and all other variables not specifically defined herein are as defined in any one of the preceding embodiments.

In a seventeenth embodiment, a compound of the disclosure is a compound of the following structural Formula IVd:

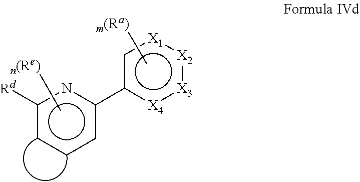

a tautomer thereof, a solvate or stereoisomer of the compound or the tautomer, or a pharmaceutically acceptable salt of the foregoing, wherein R^{b} and R^{c} join together to form an optionally substituted 5- to 7-membered heterocyclic or heteroaromatic ring; and all other variables not specifically defined herein are as defined in any one of the preceding embodiments.

In an eighteenth embodiment, a compound of the disclosure is a compound of the following structural Formula Va:

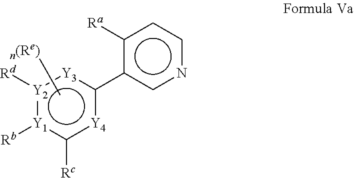

a tautomer thereof, a solvate or stereoisomer of the compound or the tautomer, or a pharmaceutically acceptable salt of the foregoing, wherein R^{a} is C₁-C₃ alkyl; and all other variables not specifically defined herein are as defined in any one of the preceding embodiments.

In a nineteenth embodiment, a compound of the disclosure is a compound of the following structural Formula VIa:

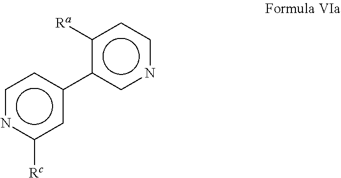

a tautomer thereof, a solvate or stereoisomer of the compound or the tautomer, or a pharmaceutically acceptable salt of the foregoing, wherein R^{c} is —OR^{s}; and all other variables not specifically defined herein are as defined in any one of the preceding embodiments.

In a twentieth embodiment, a compound of the disclosure is a compound of the following structural Formula VIb:

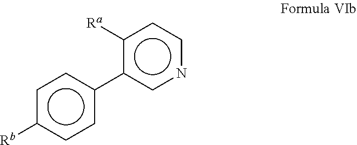

a tautomer thereof, a solvate or stereoisomer of the compound or the tautomer, or a pharmaceutically acceptable salt of the foregoing, wherein R^{b} is —OR^{s}; and all other variables not specifically defined herein are as defined in any one of the preceding embodiments.

In a twenty-first embodiment, a compound of the disclosure is a compound of the following structural Formula VIc:

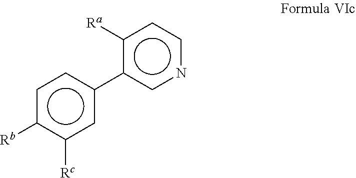

a tautomer thereof, a solvate or stereoisomer of the compound or the tautomer, or a pharmaceutically acceptable salt of the foregoing, wherein R^{b} is —OR^{s}; and R^{c} is selected from halogen, —OR^{s}, and C₁-C₃ alkyl; and all other variables not specifically defined herein are as defined in any one of the preceding embodiments.

In a twenty-second embodiment, a compound of the disclosure is a compound of the following structural Formula VId:

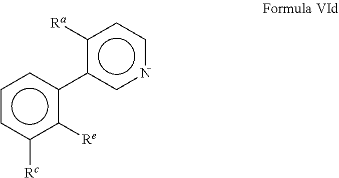

a tautomer thereof, a solvate or stereoisomer of the compound or the tautomer, or a pharmaceutically acceptable salt of the foregoing, wherein R^{c} is —OR^{s} and R^{c} is selected from halogen; and all other variables not specifically defined herein are as defined in any one of the preceding embodiments.

In a twenty-third embodiment, a compound of the disclosure is a compound of the following structural Formula VIe:

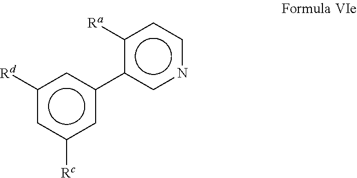

a tautomer thereof, a solvate or stereoisomer of the compound or the tautomer, or a pharmaceutically acceptable salt of the foregoing, wherein R^{c} and R^{d} are each independently selected from —OR^{s} and halogen; and all other variables not specifically defined herein are as defined in any one of the preceding embodiments.

In a twenty-fourth embodiment, a compound of the disclosure is a compound of the following structural Formula VIf:

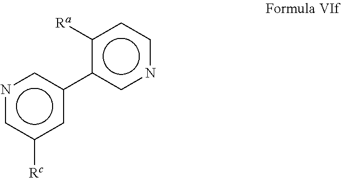

a tautomer thereof, a solvate or stereoisomer of the compound or the tautomer, or a pharmaceutically acceptable salt of the foregoing, wherein R^{c} is selected from —OR^{s} and halogen; and all other variables not specifically defined herein are as defined in any one of the preceding embodiments.

In a twenty-fifth embodiment, a compound of the disclosure is a compound of the following structural Formula VIIa:

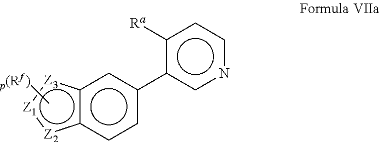

a tautomer thereof, a solvate or stereoisomer of the compound or the tautomer, or a pharmaceutically acceptable salt of the foregoing, wherein R^{a} is selected from H, —OR^{s}, halogen, —NR^{p}R^{q}, C₃-C₆ cycloalkyl, CN, and C₁-C₆ alkyl optionally substituted with 1 to 3 groups selected from halogen, —OR^{s} and —NR^{p}R^{q}, wherein R^{p} and R^{q}, for each occurrence, are independently selected from hydrogen, C₁-C₃ alkyl, phenyl, 9-10 membered aryl, 5-10 membered heteroaryl, wherein the phenyl, 9-10 membered aryl, and 5-10 membered heteroaryl of R^{p} and R^{q} are optionally substituted with 1 to 3 groups selected from —COO(C₁-C₄ alkyl), halogen, OH, —CN, —COOH, —CONH₂, —CONH(C₁-C₃ alkyl), —NH(C₁-C₃ alkyl), —O(C₁-C₃ alkyl), and C₁-C₄ alkyl; R^{f} is selected from halogen, C₁-C₄ alkyl, C₂-C₄ alkynyl, and CN, p is an integer selected from 0, 1, and 2; one, two, or three of Z₁, Z₂, and Z₃ are N, and when one or two of Z₁, Z₂, and Z₃ are N, the rest of Z₁, Z₂, and Z₃ is C; and all other variables not specifically defined herein are as defined in any one of the preceding embodiments.

In a twenty-sixth embodiment, a compound of the disclosure is a compound of the following structural Formula VIIb:

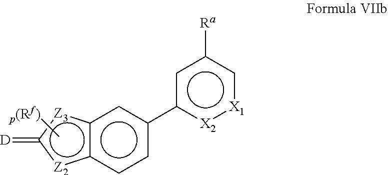

a tautomer thereof, a solvate or stereoisomer of the compound or the tautomer, or a pharmaceutically acceptable salt of the foregoing, wherein R^{a} is selected from H, —OR^{s}, halogen, —NR^{p}R^{q}, C₃-C₆ cycloalkyl, CN, and C₁-C₆ alkyl optionally substituted with 1 to 3 groups selected from halogen, —OR^{s} and —NR^{p}R^{q}, wherein R^{p} and R^{q}, for each occurrence, are independently selected from hydrogen, C₁-C₃ alkyl, phenyl, 9-10 membered aryl, 5-10 membered heteroaryl, wherein the phenyl, 9-10 membered aryl, and 5-10 membered heteroaryl of R^{p} and R^{q} are optionally substituted with 1 to 3 groups selected from —COO(C₁-C₄ alkyl), halogen, OH, —CN, —COOH, —CONH₂, —CONH(C₁-C₃ alkyl), —NH(C₁-C₃ alkyl), —O(C₁-C₃ alkyl), and C₁-C₄ alkyl; R^{f} is selected from halogen, C₁-C₄ alkyl, C₂-C₄ alkynyl, and ═O, p is an integer selected from 0, 1, and 2; Z₂ and Z₃ are each independently selected from O, N, S, and C wherein at least one of Z₂ and Z₃ is a heteroatom; D is selected from 0, S, and NH; one or two of X₁ and X₂ are N; and all other variables not specifically defined herein are as defined in any one of the preceding embodiments.

In a twenty-seventh embodiment, a compound of the disclosure is a compound of the following structural Formula VIIc:

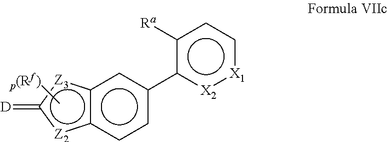

a tautomer thereof, a solvate or stereoisomer of the compound or the tautomer, or a pharmaceutically acceptable salt of the foregoing, wherein R^{a} is selected from H, —OR^{s}, halogen, —NR^{p}R^{q}, C₃-C₆ cycloalkyl, CN, and C₁-C₆ alkyl optionally substituted with 1 to 3 groups selected from halogen, —OR^{s} and —NR^{p}R^{q}, wherein R^{p} and R^{q}, for each occurrence, are independently selected from hydrogen, C₁-C₃ alkyl, phenyl, 9-10 membered aryl, 5-10 membered heteroaryl, wherein the phenyl, 9-10 membered aryl, and 5-10 membered heteroaryl of R^{p} and R^{q} are optionally substituted with 1 to 3 groups selected from —COO(C₁-C₄ alkyl), halogen, OH, —CN, —COOH, —CONH₂, —CONH(C₁-C₃ alkyl), —NH(C₁-C₃ alkyl), —O(C₁-C₃ alkyl), and C₁-C₄ alkyl; R^{f} is selected from halogen C₁-C₄ alkyl, C₂-C₄ alkynyl, and ═O, p is an integer selected from 0, 1, and 2; Z₂ and Z₃ are each independently selected from O, N, S, and C wherein at least one of Z₂ and Z₃ is a heteroatom; D is selected from 0, S, and NH; one or two of X₁ and X₂ are N; and all other variables not specifically defined herein are as defined in any one of the preceding embodiments.

In a twenty-eighth embodiment, a compound of the disclosure is a compound of the following structural Formula VIIc:

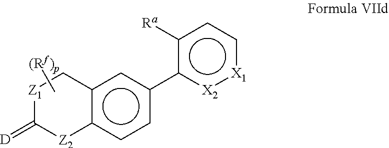

a tautomer thereof, a solvate or stereoisomer of the compound or the tautomer, or a pharmaceutically acceptable salt of the foregoing, wherein R^{a} is selected from H, —OR^{s}, halogen, —NR^{p}R^{q}, C₃-C₆ cycloalkyl, CN, and C₁-C₆ alkyl optionally substituted with 1 to 3 groups selected from halogen, —OR^{s} and —NR^{p}R^{q}, wherein R^{p} and R^{q}, for each occurrence, are independently selected from hydrogen, C₁-C₃ alkyl, phenyl, 9-10 membered aryl, 5-10 membered heteroaryl, wherein the phenyl, 9-10 membered aryl, and 5-10 membered heteroaryl of R^{p} and R^{q} are optionally substituted with 1 to 3 groups selected from —COO(C₁-C₄ alkyl), halogen, OH, —CN, —COOH, —CONH₂, —CONH(C₁-C₃ alkyl), —NH(C₁-C₃ alkyl), —O(C₁-C₃ alkyl), and C₁-C₄ alkyl; R^{f} is selected from halogen, C₁-C₄ alkyl, C₂-C₄ alkynyl, and ═O, p is an integer selected from 0, 1, and 2; Z₁ and Z₂ are each independently selected from O, N, S, and C wherein at least one of Z₁ and Z₂ is a heteroatom; D is selected from 0, NH, and S; one or two of X₁ and X₂ are N; and all other variables not specifically defined herein are as defined in any one of the preceding embodiments.

In a twenty-ninth embodiment, a compound of the disclosure is a compound of the following structural Formula VIIe:

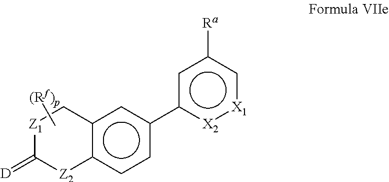

a tautomer thereof, a solvate or stereoisomer of the compound or the tautomer, or a pharmaceutically acceptable salt of the foregoing, wherein H, —OR^{s}, halogen, —NR^{p}R^{q}, C₃-C₆ cycloalkyl, CN, and C₁-C₆ alkyl optionally substituted with 1 to 3 groups selected from halogen, —OR^{s} and —NR^{p}R^{q}, wherein R^{p} and R^{q}, for each occurrence, are independently selected from hydrogen, C₁-C₃ alkyl, phenyl, 9-10 membered aryl, 5-10 membered heteroaryl, wherein the phenyl, 9-10 membered aryl, and 5-10 membered heteroaryl of R^{p} and R^{q} are optionally substituted with 1 to 3 groups selected from —COO(C₁-C₄ alkyl), halogen, OH, —CN, —COOH, —CONH₂, —CONH(C₁-C₃ alkyl), —NH(C₁-C₃ alkyl), —O(C₁-C₃ alkyl), and C₁-C₄ alkyl; R^{f} is selected from halogen, C₁-C₄ alkyl, C₂-C₄ alkynyl, and ═O, p is an integer selected from 0, 1, and 2; Z₁ and Z₂ are each independently selected from O, N, S, and C wherein at least one of Z₁ and Z₂ is a heteroatom; D is selected from O, NH, and S; one or two of X₁ and X₂ are N; and all other variables not specifically defined herein are as defined in any one of the preceding embodiments.

In a thirtieth embodiment, in a compound, tautomer, a solvate or stereoisomer of the compound or the tautomer, or pharmaceutically acceptable salt of this disclosure, R^{a} is selected from H, —OR^{s}, halogen, —NR^{p}R^{q}, C₃-C₆ cycloalkyl, CN, and C₁-C₆ alkyl optionally substituted with 1 to 3 groups selected from halogen, —OR^{s}, and —NR^{p}R^{q}, wherein R^{s}, for each occurrence, is independently selected from H, phenyl, —CFH₂, —CF₂H, —CF₃ and C₁-C₃ alkyl, and R^{p} and R^{q}, for each occurrence, are independently selected from hydrogen, C₁-C₃ alkyl, phenyl, 9-10 membered aryl, 5-10 membered heteroaryl, wherein the phenyl, 9-10 membered aryl, and 5-10 membered heteroaryl of R^{p} and R^{q} are optionally substituted with 1 to 3 groups selected from —COO(C₁-C₄ alkyl), halogen, OH, —CN, —COOH, —CONH₂, —CONH(C₁-C₃ alkyl), —NH(C₁-C₃ alkyl), —O(C₁-C₃ alkyl), and C₁-C₄ alkyl; all other variables not specifically defined herein are as defined in any one of the preceding embodiments.

In a thirty-first embodiment, in a compound, tautomer, a solvate or stereoisomer of the compound or the tautomer, or pharmaceutically acceptable salt of this disclosure, R^{a} is selected from H, —CH₃, —CH₂CH₃, —OCFH₂, —OCF₂H, —OCF₃, —OCH₃, CN, Cl, OH, NH₇, —NHCH₃, and

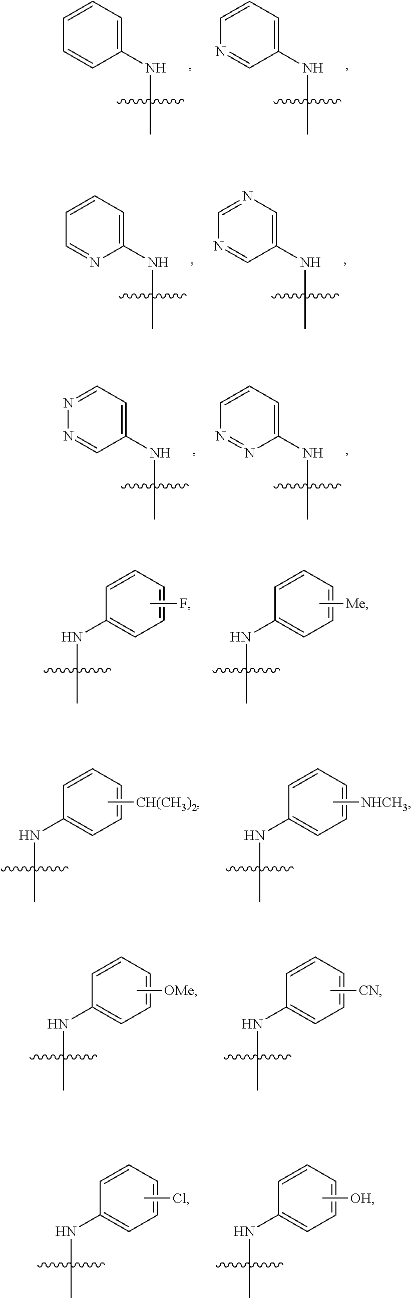

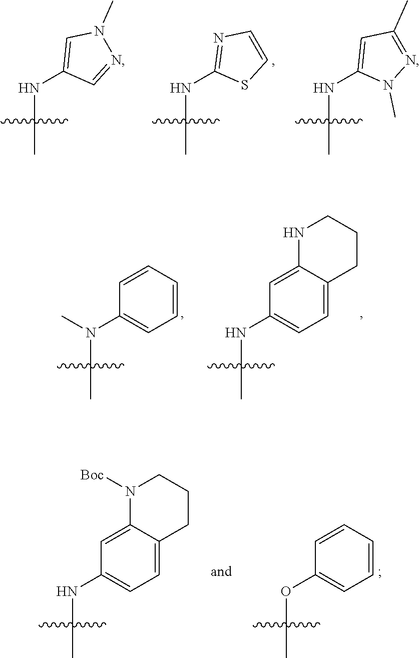

and all other variables not specifically defined herein are as defined in any one of the preceding embodiments.

In a thirty-second embodiment, in a compound, tautomer, a solvate or stereoisomer of the compound or the tautomer, or pharmaceutically acceptable salt of this disclosure, R^{b} is absent or selected from H, halogen, —C(═O)(C₁-C₃ alkyl), —NR^{p1}R^{q1}, —OR^{s}, and C₁-C₃ alkyl optionally substituted with 1 to 2 groups selected from halogen and —NR^{p1}R^{q1}; wherein R^{s}, for each occurrence, is independently selected from H, —CF₃, —CF₂H, and C₁-C₃ alkyl, and R^{p1} and R^{q1}, for each occurrence, are independently selected from hydrogen and optionally substituted C₁-C₃ alkyl; all other variables not specifically defined herein are as defined in any one of the preceding embodiments.

In a thirty-third embodiment, in a compound, tautomer, a solvate or stereoisomer of the compound or the tautomer, or pharmaceutically acceptable salt of this disclosure, R^{b} is selected from H, methyl, CH₂NH₂, —C(═O)CH₃, NH₂, F, Br, OH, and

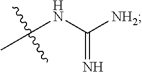 [SMILES: *NC(=N)N]

all other variables not specifically defined herein are as defined in any one of the preceding embodiments.

In a thirty-fourth embodiment, in a compound, tautomer, a solvate or stereoisomer of the compound or the tautomer, or pharmaceutically acceptable salt of this disclosure, R^{c} is selected from H, CN, —S(═O)_{w}NR^{p}R^{q}, —OR^{s}, halogen, —NR^{p1}R^{q1}, and C₁-C₃ alkyl optionally substituted with 1 to 3 groups selected from halogen and —NR^{p1}R^{q1}, wherein R^{s}, for each occurrence, is independently selected from H and C₁-C₃ alkyl, and R^{p1} and R^{q1}, for each occurrence, are independently selected from hydrogen and C₁-C₃ alkyl; all other variables not specifically defined herein are as defined in any one of the preceding embodiments.

In a thirty-fifth embodiment, in a compound, tautomer, a solvate or stereoisomer of the compound or the tautomer, or pharmaceutically acceptable salt of this disclosure, R^{c} is selected from H, methyl, ethyl, CHF₂, CF₃, F, Cl, Br, NH₂, OH, OCH₃, CN, and —S(═O)₂NH₂; all other variables not specifically defined herein are as defined in any one of the preceding embodiments.

In a thirty-sixth embodiment, in a compound, tautomer, a solvate or stereoisomer of the compound or the tautomer, or pharmaceutically acceptable salt of this disclosure, R^{d} is selected from H, CN, —S(═O)_{w}NR^{p}R^{q}, —OR^{s}, halogen, —NR^{p1}R^{q1}, and C₁-C₃ alkyl optionally substituted with 1 to 3 groups selected from halogen and —NR^{p1}R^{q1}, wherein R^{s}, for each occurrence, is independently selected from H and C₁-C₃ alkyl, and R^{p1} and R^{q1}, for each occurrence, are independently selected from hydrogen and C₁-C₃ alkyl; all other variables not specifically defined herein are as defined in any one of the preceding embodiments.

In a thirty-seventh embodiment, in a compound, tautomer, a solvate or stereoisomer of the compound or the tautomer, or pharmaceutically acceptable salt of this disclosure, R^{d} is selected from H, methyl, ethyl, CHF₂, CF₃, F, Cl, Br, NH₂, OH, OCH₃, CN, and —S(═O)₂NH₂; all other variables not specifically defined herein are as defined in any one of the preceding embodiments.

In a thirty-eighth embodiment, in a compound, tautomer, a solvate or stereoisomer of the compound or the tautomer, or pharmaceutically acceptable salt of this disclosure, R^{c} is selected from H, halogen, and C₁-C₃ alkyl optionally substituted with 1 to 3 groups selected from halogen and —NH₂; all other variables not specifically defined herein are as defined in any one of the preceding embodiments.

In a thirty-ninth embodiment, in a compound, tautomer, a solvate or stereoisomer of the compound or the tautomer, or pharmaceutically acceptable salt of this disclosure, R^{c} is selected from H, methyl, F, and Cl; all other variables not specifically defined herein are as defined in any one of the preceding embodiments.

In a fortieth embodiment, in a compound, tautomer, a solvate or stereoisomer of the compound or the tautomer, or pharmaceutically acceptable salt of this disclosure, wherein R^{b} and R^{c}, or R^{b} and R^{d}, join to form a 5- to 7-membered heterocyclic or heteroaromatic ring optionally substituted with 1 to 2 groups selected from CN, halogen, ═O, ═S, ═NH, C₁-C₃ alkyl optionally substituted with 1 to 3 groups selected from halogen, and —NR^{p}R^{q}, wherein R^{p} and R^{q}, for each occurrence, are independently selected from hydrogen, C₁-C₃ alkyl, and —C(═O)C₁-C₃ alkyl; all other variables not specifically defined herein are as defined in any one of the preceding embodiments.

In a forty-first embodiment, in a compound, tautomer, a solvate or stereoisomer of the compound or the tautomer, or pharmaceutically acceptable salt of this disclosure, wherein R^{b} and R^{c}, or R^{b} and R^{d}, join to form a structure selected from:

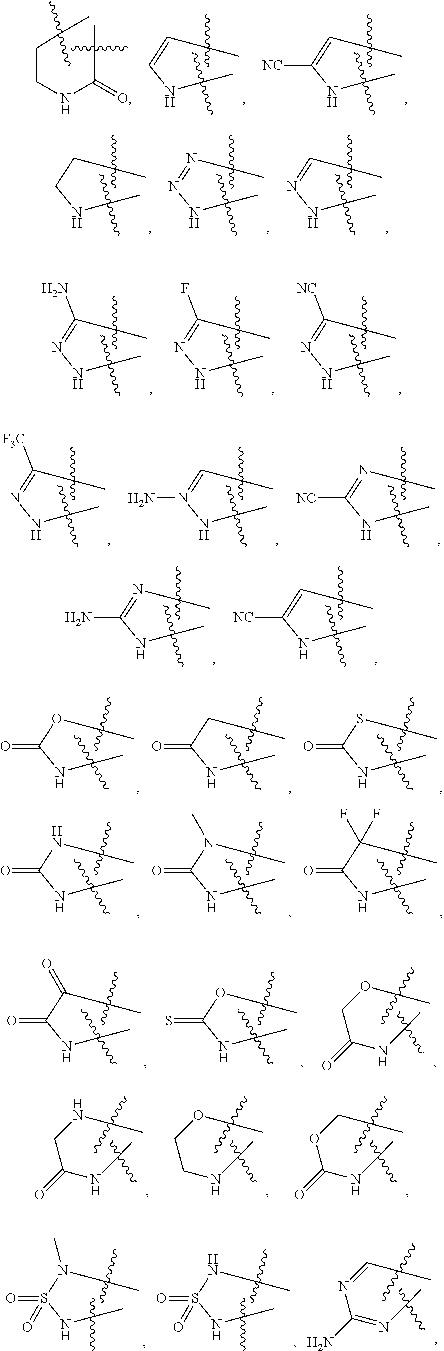

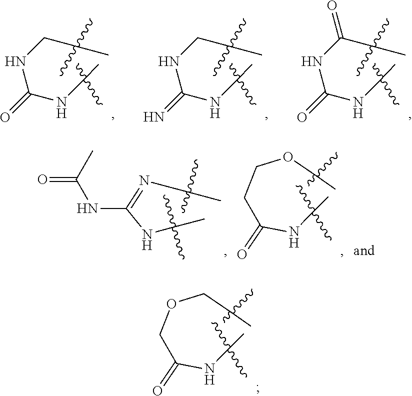

all other variables not specifically defined herein are as defined in any one of the preceding embodiments.

In a forty-second embodiment, a compound of the disclosure is a compound of the following structural Formula VIIa:

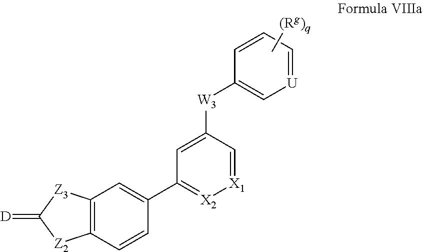

a tautomer thereof, a solvate or stereoisomer of the compound or the tautomer, or a pharmaceutically acceptable salt of the foregoing, wherein R⁹, for each occurrence, is selected from C₁-C₃ alkyl, CN, OH, —O(C₁-C₃ alkyl), —NH(C₁-C₃ alkyl), halogen, q is an integer selected from 0, 1, and 2; Z₁ and Z₂ are each independently selected from O, NH, S, and CH₂ wherein at least one of Z₂ and Z₃ is a heteroatom; D is selected from O and S; one or two of X₁ and X₂ are N; U is C or N; W₃ is O or NH.

In certain embodiments, the at least one compound of the disclosure is selected from Compounds 1 to 120 depicted in Table 1, a tautomer thereof, a solvate or stereoisomer of the compound or the tautomer, or a pharmaceutically acceptable salt of the foregoing.

TABLE 1
Compounds 1 to 120

| | |
|---|---|
| 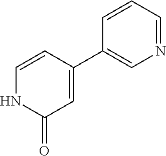 [SMILES: O=c1cc(-c2cccnc2)cc[nH]1] | 1 |
| 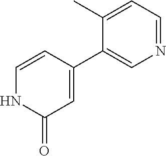 | 2 |
| 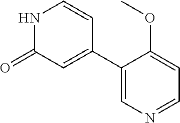 | 3 |
| 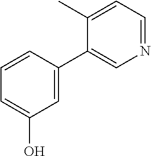 | 4 |
| 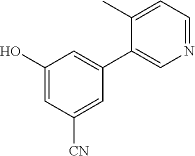 [SMILES: Cc1ccncc1-c1cc(O)cc(C#N)c1] | 5 |
| 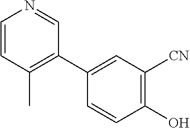 [SMILES: Cc1ccncc1-c1ccc(O)c(C#N)c1] | 6 |
| 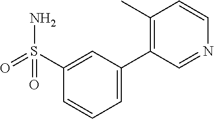 [SMILES: Cc1ccncc1-c1cccc(S(N)(=O)=O)c1] | 7 |
| 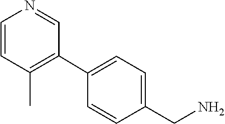 | 8 |
| 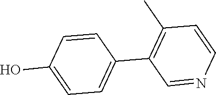 | 9 |
| 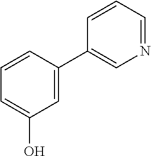 [SMILES: Oc1cccc(-c2cccnc2)c1] | 10 |
| 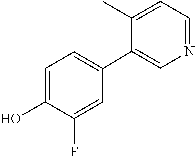 | 11 |
| 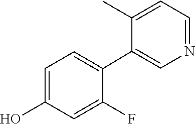 | 12 |
| 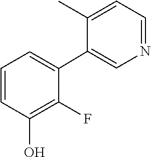 | 13 |
| 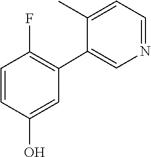 | 14 |
| 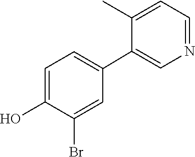 | 15 |
| 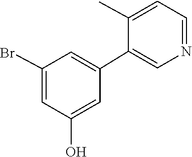 | 16 |
| 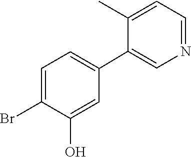 | 17 |
| 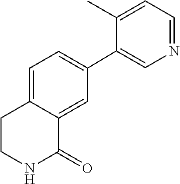 [SMILES: Cc1ccncc1-c1ccc2c(c1)C(=O)NCC2] | 18 |
| 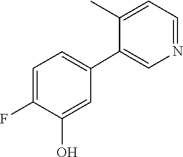 | 19 |
| 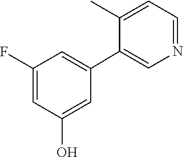 | 20 |
| 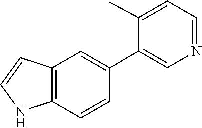 [SMILES: Cc1ccncc1-c1ccc2[nH]ccc2c1] | 21 |
| 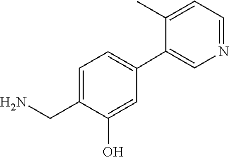 [SMILES: Cc1ccncc1-c1ccc(CN)c(O)c1] | 22 |
| 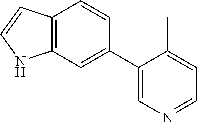 [SMILES: Cc1ccncc1-c1ccc2cc[nH]c2c1] | 23 |
| 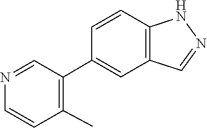 [SMILES: Cc1ccncc1-c1ccc2[nH]ncc2c1] | 24 |
| 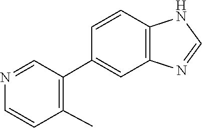 [SMILES: Cc1ccncc1-c1ccc2[nH]cnc2c1] | 25 |
| 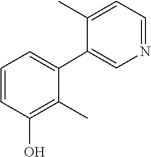 | 26 |
| 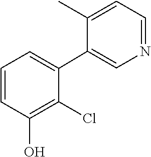 | 27 |
| 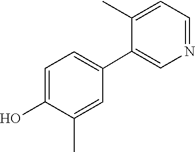 | 28 |
| 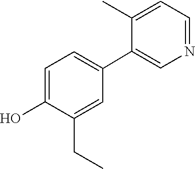 [SMILES: CCc1cc(-c2cnccc2C)ccc1O] | 29 |
| 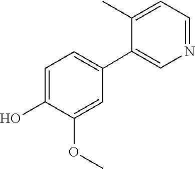 [SMILES: COc1cc(-c2cnccc2C)ccc1O] | 30 |
| 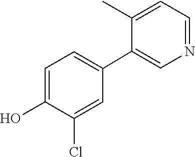 | 31 |
| 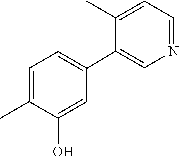 | 32 |
| 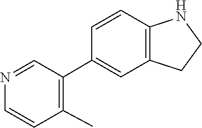 [SMILES: Cc1ccncc1-c1ccc2c(c1)CCN2] | 33 |
| 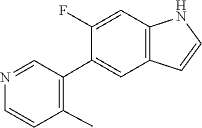 [SMILES: Cc1ccncc1-c1cc2cc[nH]c2cc1F] | 34 |
| 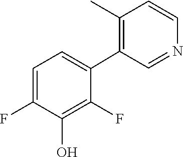 [SMILES: Cc1ccncc1-c1ccc(F)c(O)c1F] | 35 |
| 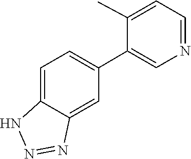 [SMILES: Cc1ccncc1-c1ccc2[nH]nnc2c1] | 36 |
| 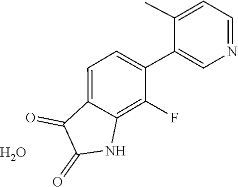 | 37 |
| 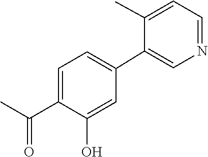 [SMILES: CC(=O)c1ccc(-c2cnccc2C)cc1O] | 38 |
| 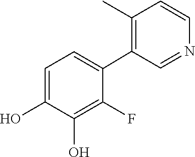 [SMILES: Cc1ccncc1-c1ccc(O)c(O)c1F] | 39 |
| 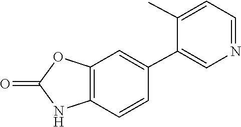 [SMILES: Cc1ccncc1-c1ccc2[nH]c(=O)oc2c1] | 40 |
| 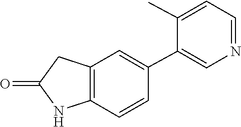 [SMILES: Cc1ccncc1-c1ccc2c(c1)CC(=O)N2] | 41 |
| 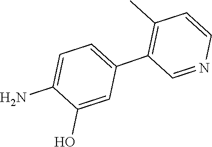 | 42 |
| 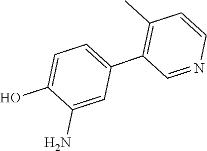 | 43 |
| 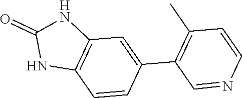 [SMILES: Cc1ccncc1-c1ccc2[nH]c(=O)[nH]c2c1] | 44 |
| 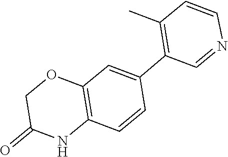 [SMILES: Cc1ccncc1-c1ccc2c(c1)OCC(=O)N2] | 45 |
| 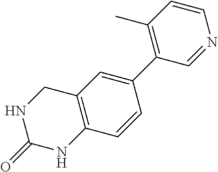 [SMILES: Cc1ccncc1-c1ccc2c(c1)CNC(=O)N2] | 46 |
| 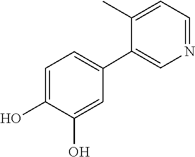 | 47 |
| 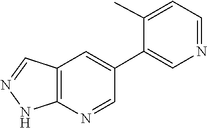 [SMILES: Cc1ccncc1-c1cnc2[nH]ncc2c1] | 48 |
| 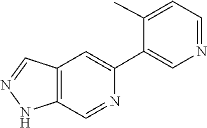 [SMILES: Cc1ccncc1-c1cc2cn[nH]c2cn1] | 49 |
| 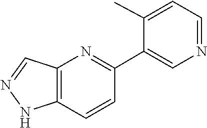 [SMILES: Cc1ccncc1-c1ccc2[nH]ncc2n1] | 50 |
| 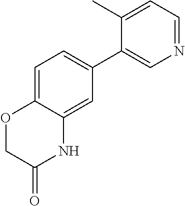 [SMILES: Cc1ccncc1-c1ccc2c(c1)NC(=O)CO2] | 51 |
| 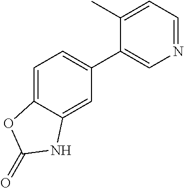 [SMILES: Cc1ccncc1-c1ccc2oc(=O)[nH]c2c1] | 52 |
| 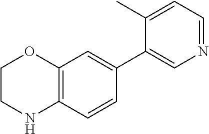 [SMILES: Cc1ccncc1-c1ccc2c(c1)OCCN2] | 53 |
| 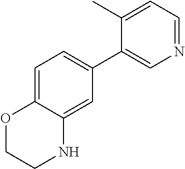 [SMILES: Cc1ccncc1-c1ccc2c(c1)NCCO2] | 54 |
| 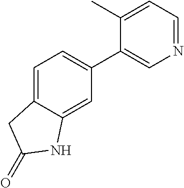 [SMILES: Cc1ccncc1-c1ccc2c(c1)NC(=O)C2] | 55 |
| 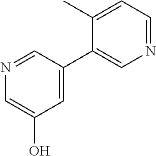 | 56 |
| 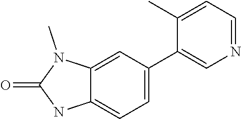 | 57 |
| 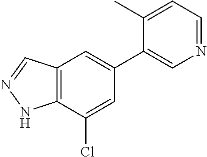 [SMILES: Cc1ccncc1-c1cc(Cl)c2[nH]ncc2c1] | 58 |
| 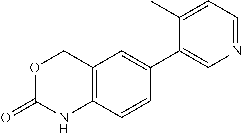 [SMILES: Cc1ccncc1-c1ccc2c(c1)COC(=O)N2] | 59 |
| 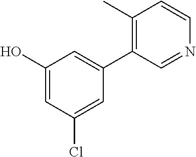 | 60 |
| 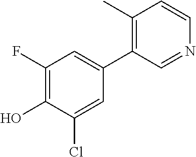 [SMILES: Cc1ccncc1-c1cc(F)c(O)c(Cl)c1] | 61 |
| 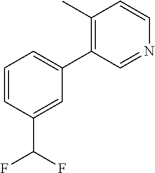 [SMILES: Cc1ccncc1-c1cccc(C(F)F)c1] | 62 |
| 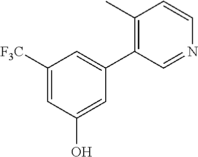 [SMILES: Cc1ccncc1-c1cc(O)cc(C(F)(F)F)c1] | 63 |
| 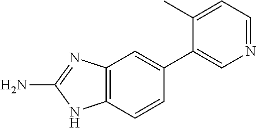 [SMILES: Cc1ccncc1-c1ccc2[nH]c(N)nc2c1] | 64 |
| 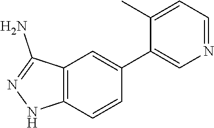 [SMILES: Cc1ccncc1-c1ccc2[nH]nc(N)c2c1] | 65 |
| 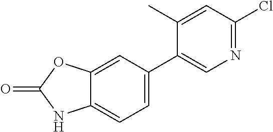 [SMILES: Cc1cc(Cl)ncc1-c1ccc2[nH]c(=O)oc2c1] | 66 |
| 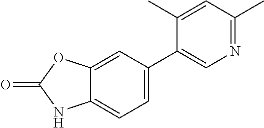 [SMILES: Cc1cc(C)c(-c2ccc3[nH]c(=O)oc3c2)cn1] | 67 |
| 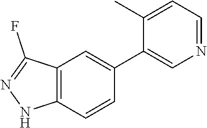 [SMILES: Cc1ccncc1-c1ccc2[nH]nc(F)c2c1] | 68 |
| 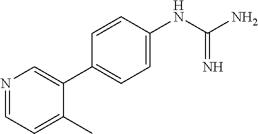 [SMILES: Cc1ccncc1-c1ccc(NC(=N)N)cc1] | 69 |
| 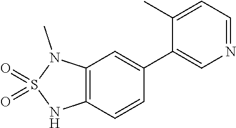 [SMILES: Cc1ccncc1-c1ccc2c(c1)N(C)S(=O)(=O)N2] | 70 |
| 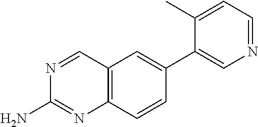 [SMILES: Cc1ccncc1-c1ccc2nc(N)ncc2c1] | 71 |
| 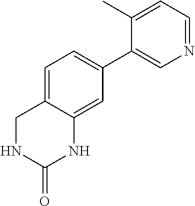 [SMILES: Cc1ccncc1-c1ccc2c(c1)NC(=O)NC2] | 72 |
| 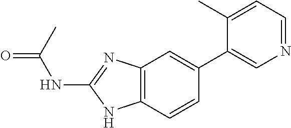 [SMILES: CC(=O)Nc1nc2cc(-c3cnccc3C)ccc2[nH]1] | 73 |
| 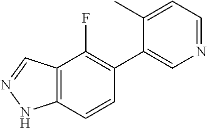 [SMILES: Cc1ccncc1-c1ccc2[nH]ncc2c1F] | 74 |
| 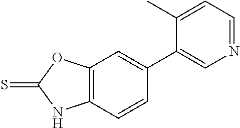 [SMILES: Cc1ccncc1-c1ccc2[nH]c(=S)oc2c1] | 75 |
| 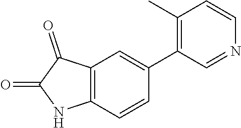 [SMILES: Cc1ccncc1-c1ccc2c(c1)C(=O)C(=O)N2] | 76 |
| 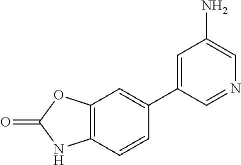 [SMILES: Nc1cncc(-c2ccc3[nH]c(=O)oc3c2)c1] | 77 |
| 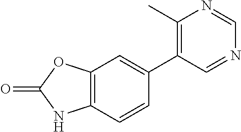 [SMILES: Cc1ncncc1-c1ccc2[nH]c(=O)oc2c1] | 78 |
| 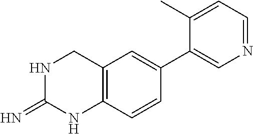 [SMILES: Cc1ccncc1-c1ccc2c(c1)CNC(=N)N2] | 79 |
| 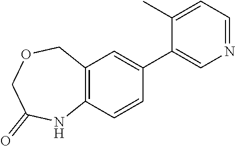 [SMILES: Cc1ccncc1-c1ccc2c(c1)COCC(=O)N2] | 80 |
| 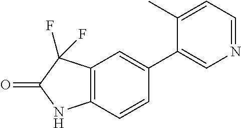 [SMILES: Cc1ccncc1-c1ccc2c(c1)C(F)(F)C(=O)N2] | 81 |
| 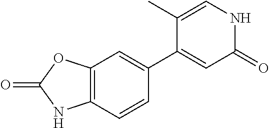 [SMILES: Cc1c[nH]c(=O)cc1-c1ccc2[nH]c(=O)oc2c1] | 82 |
| 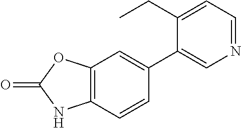 [SMILES: CCc1ccncc1-c1ccc2[nH]c(=O)oc2c1] | 83 |
| 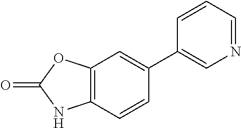 [SMILES: O=c1[nH]c2ccc(-c3cccnc3)cc2o1] | 84 |
| 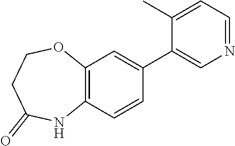 [SMILES: Cc1ccncc1-c1ccc2c(c1)OCCC(=O)N2] | 85 |
| 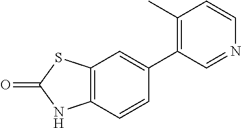 [SMILES: Cc1ccncc1-c1ccc2[nH]c(=O)sc2c1] | 86 |
| 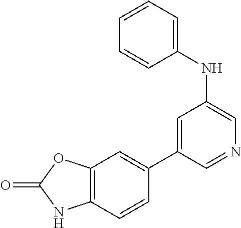 [SMILES: O=c1[nH]c2ccc(-c3cncc(Nc4ccccc4)c3)cc2o1] | 87 |
| 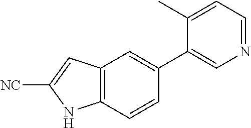 [SMILES: Cc1ccncc1-c1ccc2[nH]c(C#N)cc2c1] | 88 |
| 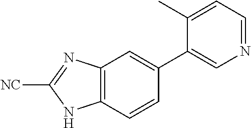 [SMILES: Cc1ccncc1-c1ccc2[nH]c(C#N)nc2c1] | 89 |
| 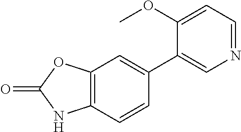 [SMILES: COc1ccncc1-c1ccc2[nH]c(=O)oc2c1] | 90 |
| 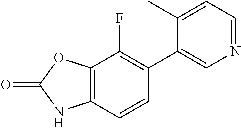 [SMILES: Cc1ccncc1-c1ccc2[nH]c(=O)oc2c1F] | 91 |
| 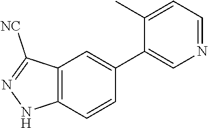 [SMILES: Cc1ccncc1-c1ccc2[nH]nc(C#N)c2c1] | 92 |
| 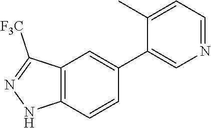 [SMILES: Cc1ccncc1-c1ccc2[nH]nc(C(F)(F)F)c2c1] | 93 |
| 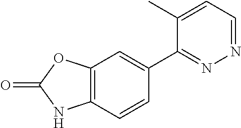 [SMILES: Cc1ccnnc1-c1ccc2[nH]c(=O)oc2c1] | 94 |
| 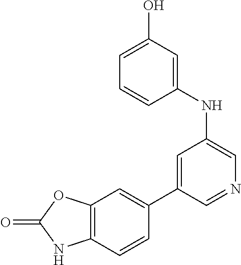 [SMILES: O=c1[nH]c2ccc(-c3cncc(Nc4cccc(O)c4)c3)cc2o1] | 95 |
| 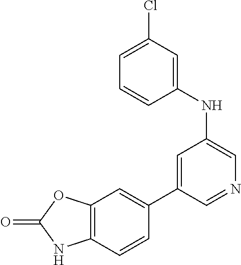 [SMILES: O=c1[nH]c2ccc(-c3cncc(Nc4cccc(Cl)c4)c3)cc2o1] | 96 |
| 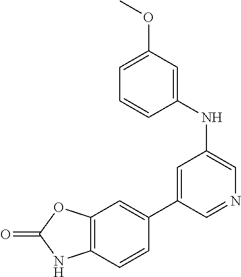 [SMILES: COc1cccc(Nc2cncc(-c3ccc4[nH]c(=O)oc4c3)c2)c1] | 97 |
| 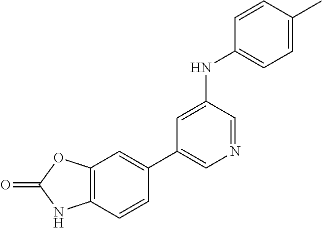 [SMILES: Cc1ccc(Nc2cncc(-c3ccc4[nH]c(=O)oc4c3)c2)cc1] | 98 |
| 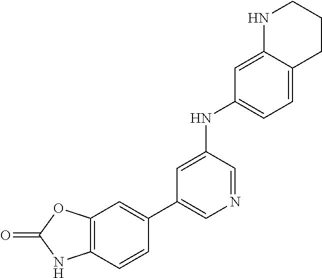 [SMILES: O=c1[nH]c2ccc(-c3cncc(Nc4ccc5c(c4)NCCC5)c3)cc2o1] | 99 |
| 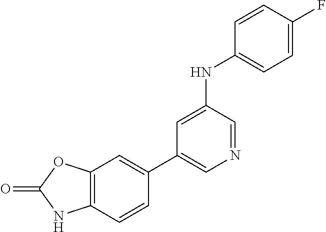 [SMILES: O=c1[nH]c2ccc(-c3cncc(Nc4ccc(F)cc4)c3)cc2o1] | 100 |
| 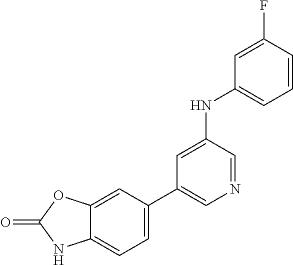 [SMILES: O=c1[nH]c2ccc(-c3cncc(Nc4cccc(F)c4)c3)cc2o1] | 101 |
| 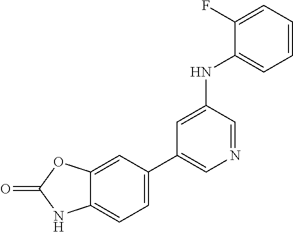 [SMILES: O=c1[nH]c2ccc(-c3cncc(Nc4ccccc4F)c3)cc2o1] | 102 |
| 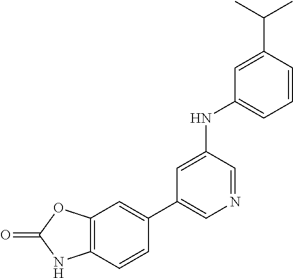 [SMILES: CC(C)c1cccc(Nc2cncc(-c3ccc4[nH]c(=O)oc4c3)c2)c1] | 103 |
| 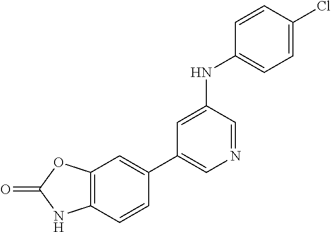 [SMILES: O=c1[nH]c2ccc(-c3cncc(Nc4ccc(Cl)cc4)c3)cc2o1] | 104 |
| 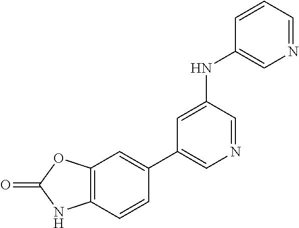 [SMILES: O=c1[nH]c2ccc(-c3cncc(Nc4cccnc4)c3)cc2o1] | 105 |
| 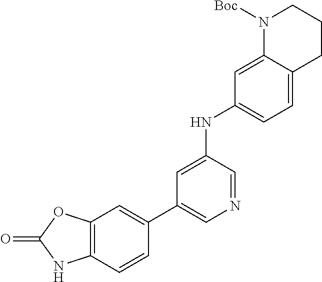 [SMILES: CC(C)(C)OC(=O)N1CCCc2ccc(Nc3cncc(-c4ccc5[nH]c(=O)oc5c4)c3)cc21] | 106 |
| 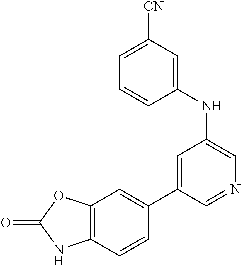 [SMILES: N#Cc1cccc(Nc2cncc(-c3ccc4[nH]c(=O)oc4c3)c2)c1] | 107 |
| 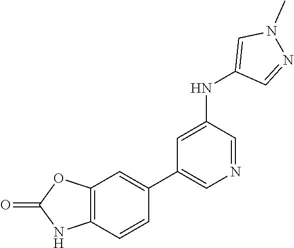 [SMILES: Cn1cc(Nc2cncc(-c3ccc4[nH]c(=O)oc4c3)c2)cn1] | 108 |
| 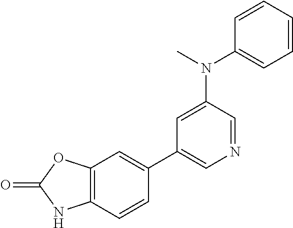 [SMILES: CN(c1ccccc1)c1cncc(-c2ccc3[nH]c(=O)oc3c2)c1] | 109 |
| 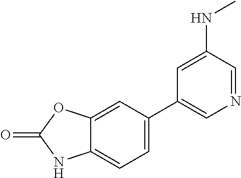 [SMILES: CNc1cncc(-c2ccc3[nH]c(=O)oc3c2)c1] | 110 |
| 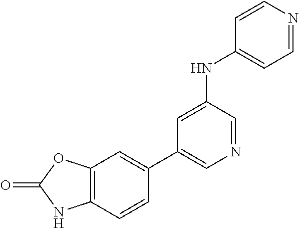 [SMILES: O=c1[nH]c2ccc(-c3cncc(Nc4ccncc4)c3)cc2o1] | 111 |
| 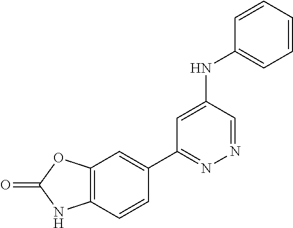 [SMILES: O=c1[nH]c2ccc(-c3cc(Nc4ccccc4)cnn3)cc2o1] | 112 |
| 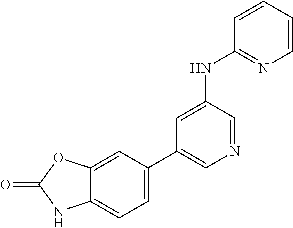 [SMILES: O=c1[nH]c2ccc(-c3cncc(Nc4ccccn4)c3)cc2o1] | 113 |
| 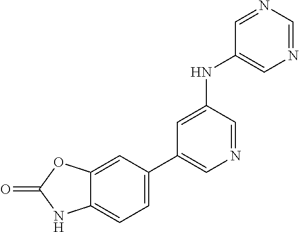 [SMILES: O=c1[nH]c2ccc(-c3cncc(Nc4cncnc4)c3)cc2o1] | 114 |
| 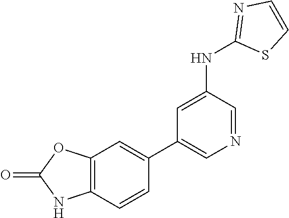 [SMILES: O=c1[nH]c2ccc(-c3cncc(Nc4nccs4)c3)cc2o1] | 115 |
| 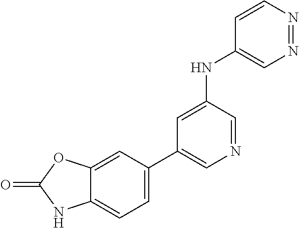 [SMILES: O=c1[nH]c2ccc(-c3cncc(Nc4ccnnc4)c3)cc2o1] | 116 |
| 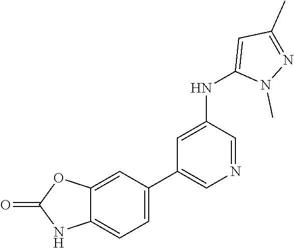 [SMILES: Cc1cc(Nc2cncc(-c3ccc4[nH]c(=O)oc4c3)c2)n(C)n1] | 117 |
| 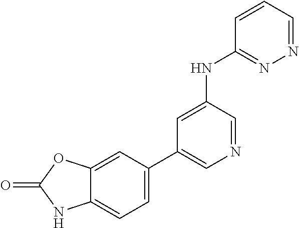 [SMILES: O=c1[nH]c2ccc(-c3cncc(Nc4cccnn4)c3)cc2o1] | 118 |
| 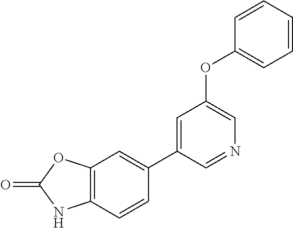 [SMILES: O=c1[nH]c2ccc(-c3cncc(Oc4ccccc4)c3)cc2o1] | 119 |
| 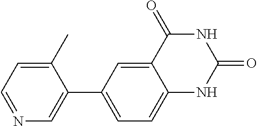 [SMILES: Cc1ccncc1-c1ccc2[nH]c(=O)[nH]c(=O)c2c1] | 120 |

Another aspect of the disclosure provides a pharmaceutical composition comprising at least one compound selected from a compound of the Formulae disclosed herein, Compounds 1 to 120, a tautomer thereof, a solvate or stereoisomer of the compound or the tautomer, or a pharmaceutically acceptable salt of the foregoing, and at least one pharmaceutically acceptable carrier.

In some embodiments, the pharmaceutically acceptable carrier is selected from pharmaceutically acceptable vehicles and pharmaceutically acceptable adjuvants. In some embodiments, the pharmaceutically acceptable carrier is chosen from pharmaceutically acceptable fillers, disintegrants, surfactants, binders, and lubricants.

It will also be appreciated that a pharmaceutical composition of this disclosure can be employed in combination therapies; that is, the pharmaceutical compositions described herein can further include an additional active pharmaceutical agent. Alternatively, a pharmaceutical composition comprising a compound selected from a compound of the Formulae disclosed herein, Compounds 1 to 120, a tautomer thereof, a solvate or stereoisomer of the compound or the tautomer, or a pharmaceutically acceptable salt of the foregoing can be administered as a separate composition concurrently with, prior to, or subsequent to, a composition comprising an additional active pharmaceutical agent.

In some embodiments, the pharmaceutically acceptable carrier may be chosen from adjuvants and vehicles. The pharmaceutically acceptable carrier, as used herein, can be chosen, for example, from any and all solvents, diluents, other liquid vehicles, dispersion aids, suspension aids, surface active agents, isotonic agents, thickening agents, emulsifying agents, preservatives, solid binders, and lubricants, which are suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988 to 1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

A compound selected from a compound of the Formulae disclosed herein, Compounds 1 to 120, a tautomer thereof, a solvate or stereoisomer of the compound or the tautomer, or a pharmaceutically acceptable salt of the foregoing, or a pharmaceutical composition disclosed herein can be administered orally in solid dosage forms, such as capsules, tablets, troches, dragées, granules and powders, or in liquid dosage forms, such as elixirs, syrups, emulsions, dispersions, and suspensions. The compound, tautomer, solvate, stereoisomer, or pharmaceutically acceptable salt described herein can also be administered parenterally, in sterile liquid dosage forms, such as dispersions, suspensions or solutions. Other dosages forms that can also be used to administer the compound, tautomer, solvate, stereoisomer, or pharmaceutically acceptable salt described herein as an ointment, cream, drops, transdermal patch or powder for topical administration, as an ophthalmic solution or suspension formation, e.g., eye drops, for ocular administration, as an aerosol spray or powder composition for inhalation or intranasal administration, or as a cream, ointment, spray or suppository for rectal or vaginal administration.

Gelatin capsules containing a compound, a tautomer thereof, a solvate or stereoisomer of the compound or the tautomer, and/or a pharmaceutically acceptable salt of the foregoing disclosed herein and powdered carriers, such as lactose, starch, cellulose derivatives, magnesium stearate, stearic acid, and the like, can also be used. Similar diluents can be used to make compressed tablets. Both tablets and capsules can be manufactured as sustained release products to provide for continuous release of medication over a period of time. Compressed tablets can be sugar coated or film coated to mask any unpleasant taste and protect the tablet from the atmosphere, or enteric coated for selective disintegration in the gastrointestinal tract.

Liquid dosage forms for oral administration can further comprise at least one agent selected from coloring and flavoring agents to increase patient acceptance.

In general, water, a suitable oil, saline, aqueous dextrose (glucose), and related sugar solutions and glycols such as propylene glycol or polyethylene glycols can be examples of suitable carriers for parenteral solutions. Solutions for parenteral administration may comprise a water-soluble salt of the at least one compound describe herein, at least one suitable stabilizing agent, and if necessary, at least one buffer substance. Antioxidizing agents such as sodium bisulfite, sodium sulfite, or ascorbic acid, either alone or combined, can be examples of suitable stabilizing agents. Citric acid and its salts and sodium EDTA can also be used as examples of suitable stabilizing agents. In addition, parenteral solutions can further comprise at least one preservative, selected, for example, from benzalkonium chloride, methyl- and propylparaben, and chlorobutanol.

A pharmaceutically acceptable carrier is, for example, selected from carriers that are compatible with active ingredients of the composition (and in some embodiments, capable of stabilizing the active ingredients) and not deleterious to the subject to be treated. For example, solubilizing agents, such as cyclodextrins (which can form specific, more soluble complexes with the at least one compound and/or at least one pharmaceutically acceptable salt disclosed herein), can be utilized as pharmaceutical excipients for delivery of the active ingredients. Examples of other carriers include colloidal silicon dioxide, magnesium stearate, cellulose, sodium lauryl sulfate, and pigments such as D&C Yellow #10. Suitable pharmaceutically acceptable carriers are described in Remington's Pharmaceutical Sciences, A. Osol.

For administration by inhalation, the compound, tautomer, solvate, stereoisomer, or pharmaceutically acceptable salt described herein may be conveniently delivered in the form of an aerosol spray presentation from pressurized packs or nebulisers. The compound, tautomer, solvate, stereoisomer, or pharmaceutically acceptable salt described herein may also be delivered as powders, which may be formulated, and the powder composition may be inhaled with the aid of an insufflation powder inhaler device. One exemplary delivery system for inhalation can be metered dose inhalation (MDI) aerosol, which may be formulated as a suspension or solution of a compound, tautomer, solvate, stereoisomer, or pharmaceutically acceptable salt described herein in at least one suitable propellant, selected, for example, from fluorocarbons and hydrocarbons.

For ocular administration, an ophthalmic preparation may be formulated with an appropriate weight percentage of a solution or suspension of the compound, tautomer, solvate, stereoisomer, or pharmaceutically acceptable salt described herein in an appropriate ophthalmic vehicle, such that the compound, tautomer, solvate, stereoisomer, or pharmaceutically acceptable salt described herein is maintained in contact with the ocular surface for a sufficient time period to allow the compound to penetrate the corneal and internal regions of the eye.

Useful pharmaceutical dosage-forms for administration of the compound, tautomer, solvate, stereoisomer, or pharmaceutically acceptable salt described herein include, but are not limited to, hard and soft gelatin capsules, tablets, parenteral injectables, and oral suspensions. In some embodiments, the pharmaceutical compositions disclosed herein may be in the form of controlled release or sustained release compositions as known in the art.

The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules, lozenges or the like in the case of solid compositions. In such compositions, the active material is usually a component ranging from about 0.1 to about 50% by weight or preferably from about 1 to about 40% by weight with the remainder being various vehicles or carriers and processing aids helpful for forming the desired dosing form. Unit dosage formulations are preferably about of 5, 10, 25, 50, 100, 250, 500, or 1,000 mg per unit. In a particular embodiment, unit dosage forms are packaged in a multipack adapted for sequential use, such as blisterpack comprising sheets of at least 6, 9 or 12 unit dosage forms.

In some embodiments, unit capsules can be prepared by filling standard two-piece hard gelatin capsules each with, for example, 100 milligrams of the compound, tautomer, solvate, stereoisomer, or pharmaceutically acceptable salt described herein in powder, 150 milligrams of lactose, 50 milligrams of cellulose, and 6 milligrams magnesium stearate.

In some embodiments, a mixture of the compound, tautomer, solvate, stereoisomer, or pharmaceutically acceptable salt described herein and a digestible oil such as soybean oil, cottonseed oil or olive oil can be prepared and injected by means of a positive displacement pump into gelatin to form soft gelatin capsules containing 100 milligrams of the active ingredient. The capsules are washed and dried.

In some embodiments, tablets can be prepared by conventional procedures so that the dosage unit comprises, for example, 100 milligrams of the compound, stereoisomers thereof, or pharmaceutically acceptable salts thereof, 0.2 milligrams of colloidal silicon dioxide, 5 milligrams of magnesium stearate, 275 milligrams of microcrystalline cellulose, 11 milligrams of starch and 98.8 milligrams of lactose. Appropriate coatings may be applied to increase palatability or delay absorption.

In some embodiments, a parenteral composition suitable for administration by injection can be prepared by stirring 1.5% by weight of the compound and/or at least an enantiomer, a diastereoisomer, or pharmaceutically acceptable salt thereof disclosed herein in 10% by volume propylene glycol. The solution is made to the expected volume with water for injection and sterilized.

In some embodiment, an aqueous suspension can be prepared for oral administration. For example, each 5 milliliters of an aqueous suspension comprising 100 milligrams of finely divided compound, stereoisomers thereof, or pharmaceutically acceptable salts thereof, 100 milligrams of sodium carboxymethyl cellulose, 5 milligrams of sodium benzoate, 1.0 grams of sorbitol solution, U.S.P., and 0.025 milliliters of vanillin can be used.

The same dosage forms can generally be used when the compound, tautomer, solvate, stereoisomer, or pharmaceutically acceptable salt described herein is administered stepwise or in conjunction with at least one other therapeutic agent. When drugs are administered in physical combination, the dosage form and administration route should be selected depending on the compatibility of the combined drugs. Thus, the term coadministration is understood to include the administration of at least two agents concomitantly or sequentially, or alternatively as a fixed dose combination of the at least two active components.

The compound, tautomer, solvate, stereoisomer, or pharmaceutically acceptable salt disclosed herein can be administered as the sole active ingredient or in combination with at least one second active ingredient.

The compound, tautomer, solvate, or stereoisomer described herein may be used per se, or in the form of their pharmaceutically acceptable salts, such as hydrochlorides, hydrobromides, acetates, sulfates, citrates, carbonates, trifluoroacetates and the like. When the compound, tautomer, solvate, stereoisomer, or pharmaceutically acceptable salt described herein contain relatively acidic functionalities, salts can be obtained by addition of the desired base, either neat or in a suitable inert solvent. Examples of pharmaceutically acceptable base addition salts include sodium, potassium, calcium, ammonium, organic amino, or magnesium salts, or the like. When the compound, tautomer, solvate, or stereoisomer described herein contain relatively basic functionalities, salts can be obtained by addition of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, for example, Berge et al., “Pharmaceutical Salts,” Journal of Pharmaceutical Science, 1977, 66, 1-19).

Neutral forms of the pharmaceutically acceptable salt described herein may be regenerated by contacting the salt with a base or acid, and isolating the parent compound in the conventional manner.

This disclosure provides prodrugs. Prodrugs of the compound, tautomer, solvate, stereoisomer, or pharmaceutically acceptable salt described herein that readily undergo chemical changes under physiological conditions to provide the compound, tautomer, solvate, stereoisomer, or pharmaceutically acceptable salt of the present disclosure. Additionally, prodrugs can be converted to the compound, tautomer, solvate, stereoisomer, or a pharmaceutically acceptable salt of the present disclosure by chemical or biochemical methods in an ex vivo environment.

For example, prodrugs can be slowly converted to the compound, tautomer, solvate, stereoisomer, or pharmaceutically acceptable salt of the present disclosure when placed in a transdermal patch reservoir with a suitable enzyme or chemical reagent. Prodrugs are often useful because, in some situations, they may be easier to administer than the parent drug. They may, for instance, be more bioavailable by oral administration than the parent drug. The prodrug may also have improved solubility in pharmacological compositions over the parent drug. A wide variety of prodrug derivatives are known in the art, such as those that rely on hydrolytic cleavage or oxidative activation of the prodrug. An example, without limitation, of a prodrug would be a compound of the present disclosure which is administered as an ester (the “prodrug”), but then is metabolically hydrolyzed to the carboxylic acid, i.e., the active entity.

Certain compound, tautomer, stereoisomer, or pharmaceutically acceptable salt of the disclosure can exist in unsolvated forms as well as solvated forms, including hydrate forms. Certain compound, tautomer, solvate, stereoisomer, or pharmaceutically acceptable salt of the disclosure may exist in multiple crystalline or amorphous forms.

Certain compound, tautomer, solvate, or pharmaceutically acceptable salt in this disclosure possesses asymmetric carbon atoms (optical centers) or double bonds; the racemates, enantiomers, diastereoisomers, geometric isomers and individual isomers are all intended to be encompassed within the scope of the present disclosure.

### III. Methods of Treatment and Uses

Another aspect of the disclosure provides a method of treating a disease or condition, comprising administering to a subject in need thereof, a therapeutically effective amount of a compound of Formulae disclosed herein, a tautomer thereof, a solvate or stereoisomer of the compound or the tautomer, or a pharmaceutically acceptable salt of the foregoing, or a pharmaceutical composition comprising any of the foregoing, wherein the disease or condition includes, but is not limited to, amyotrophic lateral sclerosis (ALS), Parkinson's disease, Parkinsonian syndromes, ischemia, stroke, herpes infection, a demyelinating disease such as multiple sclerosis, traumatic brain injury, sepsis, a chronic disease of PNS comprises inherited neuropathies, such as, but is not limited to Charcot-Marie-Tooth disease and chronic inflammatory demyelinating polyneuropathy (CIDP), an optic nerve disorder such as glaucoma, and retinal ganglion degeneration, colitis, a metabolic disease or disorder such as diabetic neuropathy, nonalcoholic fatty liver disease (NAFLD) and nonalcoholic steatohepatitis (NASH) and a peripheral neuropathy like CIPN induced by various drugs, such as, but not limited to taxanes, vinca alkaloids and proteasome inhibitors. In some embodiments, the disease or condition is caused by axonal degeneration or neuronal cells damage.

In another aspect, disclosed herein is a compound, tautomer, solvate, stereoisomer, or pharmaceutically acceptable salt as described herein, including a compound of the Formulae disclosed herein, Compounds 1 to 120, a tautomer thereof, a solvate or stereoisomer of the compound or the tautomer, or a pharmaceutically acceptable salt of the foregoing, or a pharmaceutical composition thereof, for use as a medicament.

In another aspect, disclosed herein is use of a compound, tautomer, solvate, stereoisomer, or pharmaceutically acceptable salt as described herein, including a compound of the Formulae disclosed herein, Compounds 1 to 120, a tautomer thereof, a solvate or stereoisomer of the compound or the tautomer, or a pharmaceutically acceptable salt of the foregoing, or a pharmaceutical composition thereof, for the manufacture of a medicament for treating a disease or condition includes, but is not limited to, amyotrophic lateral sclerosis (ALS), Parkinson's disease, Parkinsonian syndromes, ischemia, stroke, herpes infection, a demyelinating disease such as multiple sclerosis, traumatic brain injury, sepsis, a chronic disease of PNS comprises inherited neuropathies, such as, but is not limited to Charcot-Marie-Tooth disease and chronic inflammatory demyelinating polyneuropathy (CIDP), an optic nerve disorder such as glaucoma, and retinal ganglion degeneration, colitis, a metabolic disease or disorder such as diabetic neuropathy, nonalcoholic fatty liver disease (NAFLD) and nonalcoholic steatohepatitis (NASH) and a peripheral neuropathy like CIPN induced by various drugs, such as, but is not limited to taxanes, vinca alkaloids and proteasome inhibitors. In some embodiments, the disease or condition is caused by axonal degeneration or neuronal cell damage.

In a further aspect of this disclosure, a compound, tautomer, solvate, stereoisomer, or pharmaceutically acceptable salt as described herein, including a compound of the Formulae disclosed herein, Compounds 1 to 120, a tautomer thereof, a solvate or stereoisomer of the compound or the tautomer, or a pharmaceutically acceptable salt of the foregoing, or a pharmaceutical composition thereof, is for use in treating a disease or condition includes, but is not limited to, amyotrophic lateral sclerosis (ALS), Parkinson's disease, Parkinsonian syndromes, ischemia, stroke, herpes infection, a demyelinating disease such as multiple sclerosis, traumatic brain injury, sepsis, a chronic disease of PNS comprises inherited neuropathies, such as, but is not limited to Charcot-Marie-Tooth disease and chronic inflammatory demyelinating polyneuropathy (CIDP), an optic nerve disorder such as glaucoma, and retinal ganglion degeneration, colitis, a metabolic disease or disorder such as diabetic neuropathy, nonalcoholic fatty liver disease (NAFLD) and nonalcoholic steatohepatitis (NASH) and a peripheral neuropathy like CIPN induced by various drugs, such as, but is not limited to taxanes, vinca alkaloids and proteasome inhibitors. In some embodiments, the disease or condition is caused by axonal degeneration or neuronal cell damage.

Another aspect of the disclosure provides a method of inhibiting or preventing axonal degeneration, comprising administering to a subject in need thereof, a therapeutically effective amount of a compound of Formulae disclosed herein, a tautomer thereof, a solvate or stereoisomer of the compound or the tautomer, or a pharmaceutically acceptable salt of the foregoing, or a pharmaceutical composition comprising any of the foregoing.

In another aspect, disclosed herein is use of a compound, tautomer, solvate, stereoisomer, or pharmaceutically acceptable salt as described herein, including a compound of the Formulae disclosed herein, Compounds 1 to 120, a tautomer thereof, a solvate or stereoisomer of the compound or the tautomer, or a pharmaceutically acceptable salt of the foregoing, or a pharmaceutical composition thereof, for the manufacture of a medicament for inhibiting or preventing axonal degeneration or neuronal cells damage.

In a further aspect of this disclosure, a compound, tautomer, solvate, stereoisomer, or pharmaceutically acceptable salt as described herein, including a compound of the Formulae disclosed herein, Compounds 1 to 120, a tautomer thereof, a solvate or stereoisomer of the compound or the tautomer, or a pharmaceutically acceptable salt of the foregoing, or a pharmaceutical composition thereof, is for use in inhibiting or preventing axonal degeneration or neuronal cells damage.

Another aspect of the disclosure provides a method of modulating, e.g., inhibiting, SARM1 in a subject in need thereof, comprising administering to the subject, a therapeutically effective amount of a compound of Formulae disclosed herein, a tautomer thereof, a solvate or stereoisomer of the compound or the tautomer, or a pharmaceutically acceptable salt of the foregoing, or a pharmaceutical composition comprising any of the foregoing.

In another aspect, disclosed herein is use of a compound, tautomer, solvate, stereoisomer, or pharmaceutically acceptable salt as described herein, including a compound of the Formulae disclosed herein, Compounds 1 to 120, a tautomer thereof, a solvate or stereoisomer of the compound or the tautomer, or a pharmaceutically acceptable salt of the foregoing, or a pharmaceutical composition thereof, for modulating, e.g., inhibiting, SARM1 in a subject in need thereof.

In another aspect of this disclosure, a compound, tautomer, a solvate or stereoisomer of the compound or the tautomer, or pharmaceutically acceptable salt as described herein, including a compound of the Formulae disclosed herein, Compounds 1 to 120, a tautomer thereof, a solvate or stereoisomer of the compound or the tautomer, or a pharmaceutically acceptable salt of the foregoing, or a pharmaceutical composition thereof, is for use in modulating, e.g., inhibiting, SARM1 in a subject in need thereof by contacting the subject with the compound, tautomer, a solvate or stereoisomer of the compound or the tautomer, pharmaceutically acceptable salt, or pharmaceutical composition.

A compound of the Formulae disclosed herein, Compounds 1 to 120, a tautomer thereof, a solvate or stereoisomer of the compound or the tautomer, or a pharmaceutically acceptable salt of the foregoing, or a pharmaceutical composition thereof may be administered once daily, twice daily, or three times daily, for example, for the treatment of a disease or condition, includes, but is not limited to, amyotrophic lateral sclerosis (ALS), Parkinson's disease, Parkinsonian syndromes, ischemia, stroke, herpes infection, a demyelinating disease such as multiple sclerosis, traumatic brain injury, sepsis, a chronic disease of PNS comprises inherited neuropathies, such as, but is not limited to Charcot-Marie-Tooth disease and chronic inflammatory demyelinating polyneuropathy (CIDP), an optic nerve disorder such as glaucoma, and retinal ganglion degeneration, colitis, a metabolic disease or disorder such as diabetic neuropathy, nonalcoholic fatty liver disease (NAFLD) and nonalcoholic steatohepatitis (NASH) and a peripheral neuropathy like CIPN induced by various drugs, such as, but is not limited to taxanes, vinca alkaloids and proteasome inhibitors. In some embodiments, the disease or condition is caused by axonal degeneration or neuronal cells damage.

A compound of the Formulae disclosed herein, Compounds 1 to 120, a tautomer thereof, a solvate or stereoisomer of the compound or the tautomer, or a pharmaceutically acceptable salt of the foregoing, or a pharmaceutical composition thereof may be administered, for example, various manners, such as orally, topically, rectally, parenterally, by inhalation spray, or via an implanted reservoir, although the most suitable route in any given case will depend on the particular host, and nature and severity of the conditions for which the active ingredient is being administered. The term “parenteral” as used herein includes subcutaneous, intracutaneous, intravenous, intramuscular, intraarticular, intraarterial, intrasynovial, intrasternal, intrathecal, intralesional and intracranial injection or infusion techniques. The compositions disclosed herein may be conveniently presented in unit dosage form and prepared by any of the methods well known in the art. Parenteral administration can be by continuous infusion over a selected period of time. Other forms of administration contemplated in this disclosure are as described in International Patent Application Nos. WO 2013/075083, WO 2013/075084, WO 2013/078320, WO 2013/120104, WO 2014/124418, WO 2014/151142, and WO 2015/023915.

The contacting is generally effected by administering to the subject an effective amount of one or more compounds, tautomers, solvates, stereoisomers, and pharmaceutically acceptable salt disclosed herein.

Generally, administration is adjusted to achieve a therapeutic dosage of about 0.1 to 50, preferably 0.5 to 10, more preferably 1 to 10 mg/kg, though optimal dosages are compound specific, and generally empirically determined for each compound.

The dosage administered will be dependent on factors, such as the age, health and weight of the recipient, the extent of disease, type of concurrent treatment, if any, frequency of treatment, and the nature of the effect desired. In general, a daily dosage of the active ingredient can vary, for example, from 0.1 to 2000 milligrams per day. For example, 10-500 milligrams once or multiple times per day may be effective to obtain the desired results.

In some embodiments, 2 mg to 1500 mg or 5 mg to 1000 mg of a compound of the Formulae disclosed herein, Compounds 1 to 120, a tautomer thereof, a solvate or stereoisomer of the compound or the tautomer, or a pharmaceutically acceptable salt of the foregoing, or a pharmaceutical composition thereof are administered once daily, twice daily, or three times daily. The compound, tautomer, solvate, stereoisomer, or pharmaceutically acceptable salt described herein is administered for morning/daytime dosing, with off period at night.

### EXAMPLES

In order that the disclosure described herein may be more fully understood, the following examples are disclosed herein. It should be understood that these examples are for illustrative purposes only and are not to be construed as limiting this disclosure in any way.

### Example 1. Synthesis of Exemplary Compounds

The compounds of the disclosure, selected from a compound of the Formulae depicted herein, a tautomer thereof, a solvate or stereoisomer of the compound or the tautomer, or a pharmaceutically acceptable salt of the foregoing, can be made according to standard chemical practices or as illustrated herein, including the following synthetic schemes for Compounds 1 to 120 as representative examples of Formula I.

### [3,4′-bipyridin]-2′(1′H)-one (1)

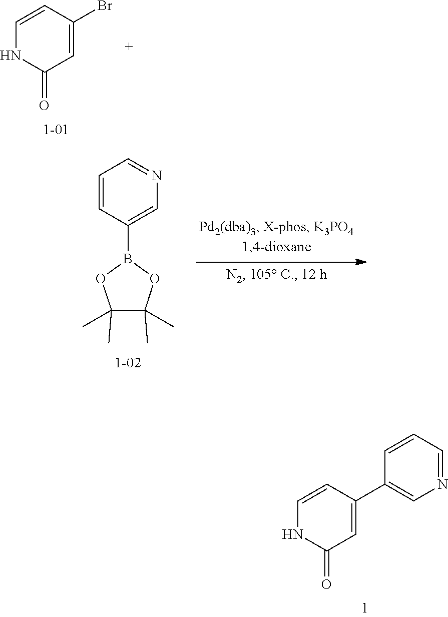

4-Bromopyridin-2(1H)-one 1-01 (174 mg, 1.0 mmol), 3-(4,4,5,5-tetramethyl-1,3,2-dioxaborolan-2-yl)pyridine 1-02 (246 mg, 1.2 mmol), Pd₂(dba)₃ (92 mg, 0.1 mmol), X-phos (95 mg, 0.2 mmol) and aqueous K₃PO₄ (5 M, 1 mL, 5.0 mmol) were placed in 1,4-dioxane (2 mL) under N₂. The mixture was stirred at 105° C. for 12 hours. The reaction mixture was cooled to room temperature, extracted with EA, washed with brine, dried over Na₂SO₄ and concentrated in vacuo. The crude product was purified by silica gel chromatography to give compound 1 (24 mg, yield: 13.9%) as a white solid. Mass (m/z): 173.1 [M+H]⁺. ¹H NMR (400 MHz, DMSO-d₆) δ 11.70 (s, 1H), 8.91 (d, J=2.4 Hz, 1H), 8.65 (dd, J=4.7, 1.6 Hz, 1H), 8.12 (dt, J=8.0, 2.1 Hz, 1H), 7.54-7.46 (m, 2H), 6.69 (d, J 10=1.8 Hz, 1H), 6.57 (dd, J=6.8, 1.9 Hz, 1H).

### 4-methyl-[3,4′-bipyridin]-2′(1′H)-one (2)

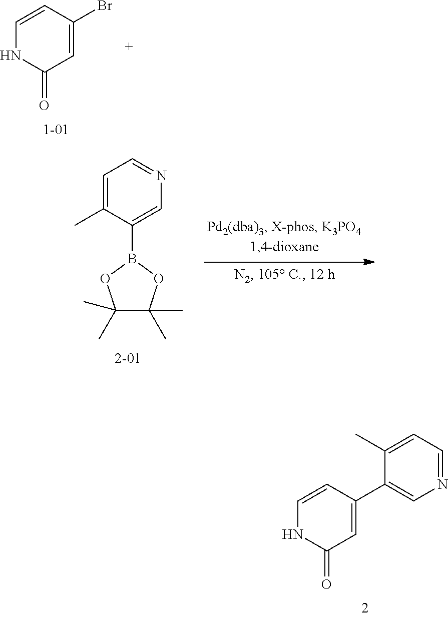

The titled compound 2 was prepared in a 4.3% yield according to the procedure outlined for compound 1. Mass (m/z): 187.1 [M+H]⁺. ¹H NMR (400 MHz, DMSO-d₆) δ 8.62 (dd, J=5.4, 1.6 Hz, 1H), 8.57 (d, J=2.0 Hz, 1H), 7.65 (d, J=5.4 Hz, 1H), 7.50 (d, J=6.7 Hz, 1H), 6.37 (d, J=1.8 Hz, 1H), 6.26 (dd, J=6.7, 1.8 Hz, 1H), 2.40 (s, 3H).

### 4-methoxy-[3,4′-bipyridin]-2′(1′H)-one (3)

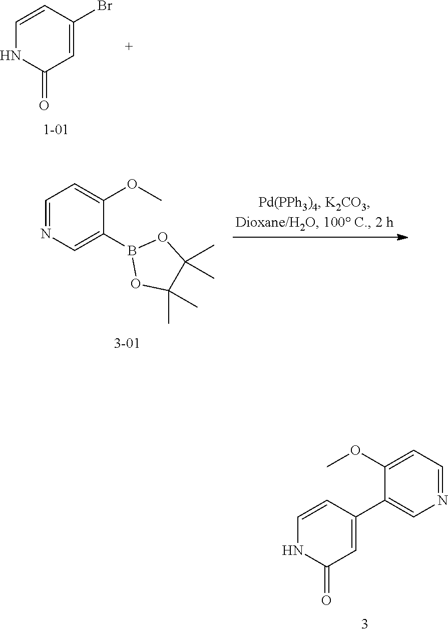 [SMILES: O=c1cc(Br)cc[nH]1 ; COc1ccncc1B1OC(C)(C)C(C)(C)O1 ; COc1ccncc1-c1cc[nH]c(=O)c1]

A mixture of 4-Bromopyridin-2(1H)-one 1-01 (150 mg, 0.86 mmol), Pd(PPh₃)₄ (149 mg, 0.13 mmol), K₂CO₃ (357 mg, 2.58 mmol) and 4-methoxy-3-(4,4,5,5-tetramethyl-1,3,2-dioxaborolan-2-yl)pyridine 3-01 (243 mg, 1.03 mmol) in dioxane (12 ml) and H₂O (3 ml) was stirred under N₂ at 100° C. for 2 h. The reaction mixture was cooled to rt and diluted with water. The aqueous phase was extracted with ethyl acetate. The combined organic extracts were washed with brine and dried over Na₂SO₄. The solvent was removed under vacuum and purified by prep-TLC to give compound 3 (20 mg, yield: 11%) as a white solid. Mass (m/z): 203.1 [M+H]⁺. ¹H NMR (400 MHz, DMSO-d6) δ 11.61 (s, 1H), 8.49 (d, J=5.8 Hz, 11H), 8.38 (s, 11H), 7.39 (dd, J=6.7, 0.7 Hz, 11H), 7.18 (d, J=5.8 Hz, 11H), 6.43 (dd, J=1.8, 0.7 Hz, 1H), 6.33 (dd, J=6.8, 1.8 Hz, 1H), 3.88 (s, 3H).

### 3-(4-methylpyridin-3-yl)phenol (4)

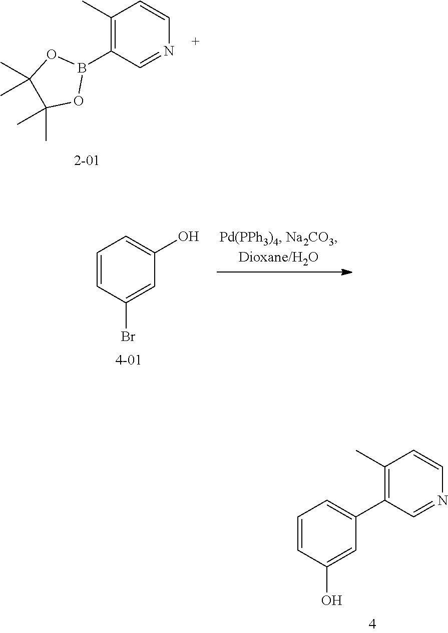 [SMILES: Cc1ccncc1B1OC(C)(C)C(C)(C)O1 ; Oc1cccc(Br)c1 ; Cc1ccncc1-c1cccc(O)c1]

The titled compound 4 was prepared in a yield of 35.10% according to the procedure outlined for compound 3. Mass (m/z): 186.1 [M+H]⁺. ¹H NMR (400 MHz, DMSO-d₆) δ 9.59 (s, 1H), 8.41 (d, J=5.0 Hz, 1H), 8.33 (s, 1H), 7.32 (d, J=5.0 Hz, 1H), 7.27 (t, J=7.8 Hz, 1H), 6.84-6.68 (m, 3H), 2.25 (s, 3H).

### 3-hydroxy-5-(4-methylpyridin-3-yl)benzonitrile (5)

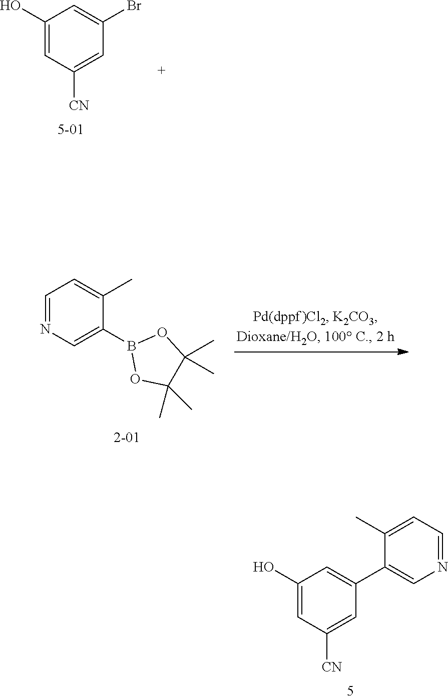 [SMILES: N#Cc1cc(O)cc(Br)c1 ; Cc1ccncc1B1OC(C)(C)C(C)(C)O1 ; Cc1ccncc1-c1cc(O)cc(C#N)c1]

A mixture of 3-bromo-5-hydroxybenzonitrile 5-01 (150 mg, 0.76 mmol), Pd(dppf)Cl₂ (83 mg, 0.11 mmol), K₂CO₃ (314 mg, 2.27 mmol) and 4-methyl-3-(4,4,5,5-tetramethyl-1,3,2-dioxaborolan-2-yl)pyridine 2-01 (191 mg, 0.83 mmol) in dioxane (10 mL) and H₂O (2 mL) was stirred under N₂ at 100° C. for 2 h. The reaction mixture was cooled to rt and diluted with water. The aqueous phase was extracted with EA. The combined organic extracts were washed with brine and dried over Na₂SO₄. The solvent was removed under vacuum and purified by flash to afford 3-hydroxy-5-(4-methylpyridin-3-yl)benzonitrile compound 5 (63 mg, yield: 40%) as a white solid. Mass (m/z): 211.1 [M+H]⁺. ¹H NMR (400 MHz, DMSO-d₆) δ 10.46 (s, 1H), 8.45 (d, J=5.0 Hz, 1H), 8.37 (s, 1H), 7.35 (d, J=4.9 Hz, 11H), 7.32 (t, J=1.5 Hz, 11H), 7.22-7.17 (m, 11H), 7.08 (t, J=2.0 Hz, 1H), 2.25 (s, 3H).

### 2-hydroxy-5-(4-methylpyridin-3-yl)benzonitrile (6)

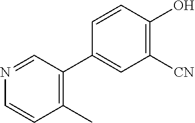 [SMILES: Cc1ccncc1-c1ccc(O)c(C#N)c1]

The titled compound 6 was prepared in a yield of 24.9% according to the procedure outlined for compound 5. Mass (m/z): 211.1 [M+H]⁺. ¹H NMR (400 MHz, DMSO-d₆) δ 11.57 (s, 1H), 8.86-8.39 (m, 2H), 7.79 (t, J=3.6 Hz, 2H), 7.62 (dd, J=8.6, 2.3 Hz, 1H), 7.16 (d, J=8.6 Hz, 1H), 2.43 (s, 3H).

### 3-(4-methylpyridin-3-yl)benzenesulfonamide (7)

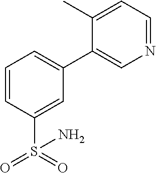 [SMILES: Cc1ccncc1-c1cccc(S(N)(=O)=O)c1]

The titled compound 7 was prepare in a yield of 43% according to the procedure outlined for compound 5. Mass (m/z): 249.1 [M+H]⁺. ¹H NMR (400 MHz, DMSO-d₆) δ 8.50 (s, 1H), 8.42 (s, 1H), 7.88 (dt, J=7.2, 1.9 Hz, 1H), 7.83 (t, J=1.7 Hz, 1H), 7.73-7.65 (m, 2H), 7.44 (s, 2H), 7.40 (d, J=5.0 Hz, 1H), 2.28 (s, 3H).

### (4-(4-methylpyridin-3-yl)phenyl)methanamine (8)

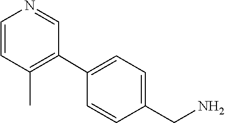

The titled compound 8 was prepared in a yield of 44.4% according to the procedure outlined for compound 5. Mass (m/z): 198.9 [M+H]⁺. ¹H NMR (400 MHz, Chloroform-d) δ 8.44 (d, J=5.0 Hz, 1H), 8.38 (s, 1H), 7.43 (d, J=8.0 Hz, 2H), 7.30 (d, J=8.0 Hz, 2H), 7.19 (d, J=5.0 Hz, 1H), 3.97 (s, 2H), 2.29 (s, 3H).

### 4-(4-methylpyridin-3-yl)phenol (9)

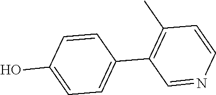

The titled compound 9 was prepared in a yield of 45.8% according to the procedure outlined for compound 5. Mass (m/z): 185.9 [M+H]⁺. ¹H NMR (400 MHz, Chloroform-d) δ 8.42 (s, 2H), 7.26-7.23 (m, 1H), 7.19 (d, J=8.5 Hz, 2H), 6.96 (d, J=8.5 Hz, 2H), 2.34 (s, 3H).

### 3-(pyridin-3-yl)phenol 10

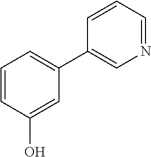 [SMILES: Oc1cccc(-c2cccnc2)c1]

The titled compound 10 was prepared in a yield of 54.3% according to the procedure outlined for compound 5. Mass (m/z): 171.9 [M+H]⁺. ¹H NMR (400 MHz, DMSO-d₆) δ 9.68 (s, 1H), 8.85-8.79 (m, 1H), 8.55 (d, J=3.6 Hz, 1H), 7.99 (d, J=7.9 Hz, 1H), 7.46 (dd, J=7.7, 4.8 Hz, 1H), 7.29 (t, J=7.8 Hz, 1H), 7.13-7.04 (m, 2H), 6.83 (dd, J=7.9, 1.6 Hz, 1H).

### 2-fluoro-4-(4-methylpyridin-3-yl)phenol (11)

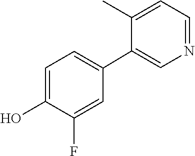

The titled compound 11 was prepared in a yield of 18.2% according to the procedure outlined for compound 5. Mass (m/z): 204.2 [M+H]⁺. ¹H NMR (400 MHz, DMSO-d₆) δ 8.43 (d, J=5.0 Hz, 1H), 8.31 (s, 1H), 8.12 (s, 1H), 7.39-7.28 (m, 1H), 7.20-6.90 (m, 2H), 6.69 (m, 1H), 2.13 (s, 3H).

### 3-fluoro-4-(4-methylpyridin-3-yl)phenol (12)

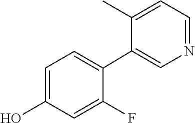

The titled compound 12 was prepared in a yield of 19.9% according to the procedure outlined for compound 5. Mass (m/z): 204.1 [M+H]⁺. ¹H NMR (400 MHz, Methanol-d₄) δ 8.39 (d, J=5.1 Hz, 1H), 8.29 (s, 1H), 7.38 (d, J=5.2 Hz, 11H), 7.12 (t, J=8.6 Hz, 11H), 6.80-6.74 (m, 11H), 6.74-6.64 (m, 1H), 2.27 (s, 3H).

### 2-fluoro-3-(4-methylpyridin-3-yl)phenol (13)

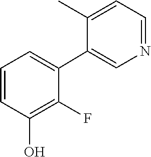

The titled compound 13 was prepared in a yield of 24.8% according to the procedure outlined for compound 5. Mass (m/z): 204.1 [M+H]⁺. ¹H NMR (400 MHz, DMSO-d₆) δ 10.02 (s, 1H), 8.41-8.21 (m, 2H), 7.39-7.12 (m, 2H), 7.12-6.89 (m, 2H), 2.23 (s, 3H).

### 4-fluoro-3-(4-methylpyridin-3-yl)phenol (14)

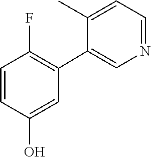

The titled compound 14 was prepared in a yield of 11.5% according to the procedure outlined for compound 5. Mass (m/z): 203.9 [M+H]⁺. ¹H NMR (400 MHz, Methanol-d₄) δ 8.42 (d, J=5.2 Hz, 1H), 8.31 (s, 1H), 7.40 (d, J=5.2 Hz, 1H), 7.07 (t, J=9.2 Hz, 1H), 6.87 (m, 1H), 6.69 (dd, J=6.0, 3.0 Hz, 1H), 2.28 (s, 3H).

### 2-bromo-4-(4-methylpyridin-3-yl)phenol (15)

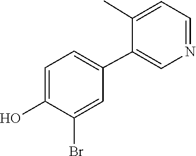

The titled compound 15 was prepared in a yield of 12.3% according to the procedure outlined for compound 5. Mass (m/z): 263.7 [M+H]⁺. ¹H NMR (400 MHz, DMSO-d₆) δ 10.46 (s, 1H), 8.39 (d, J=5.0 Hz, 1H), 8.33 (s, 11H), 7.51 (d, J=2.2 Hz, 11H), 7.30 (d, J=4.9 Hz, 11H), 7.22 (dd, J=8.3, 2.2 Hz, 1H), 7.04 (d, J=8.3 Hz, 1H), 2.25 (s, 3H).

### 3-bromo-5-(4-methylpyridin-3-yl)phenol (16)

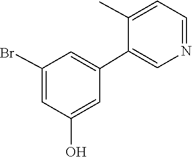

The titled compound 16 was prepared in a yield of 6.7% according to the procedure outlined for compound 5. Mass (m/z): 263.7 [M+H]⁺. ¹H NMR (400 MHz, DMSO-d₆) δ 10.14 (s, 1H), 8.43 (d, J=5.0 Hz, 1H), 8.33 (s, 1H), 7.32 (d, J=5.0 Hz, 1H), 7.00 (t, J=2.0 Hz, 1H), 6.99 (t, J=1.6 Hz, 1H), 6.75-6.73 (m, 1H), 2.25 (s, 3H).

### 2-bromo-5-(4-methylpyridin-3-yl)phenol (17)

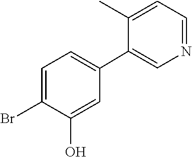

The titled compound 17 was prepared in a yield of 14.2% according to the procedure outlined for compound 5. Mass (m/z): 263.7 [M+H]⁺. ¹H NMR (400 MHz, DMSO-d₆) δ 10.65 (s, 1H), 8.42 (d, J=5.0 Hz, 1H), 8.33 (s, 1H), 7.55 (d, J=8.1 Hz, 1H), 7.32 (d, J=5.0 Hz, 1H), 6.90 (d, J=2.0 Hz, 1H), 6.73 (dd, J=8.1, 1.8 Hz, 1H), 2.24 (s, 3H).

### 7-(4-methylpyridin-3-yl)-3,4-dihydroisoquinolin-1(2H)-one (18)

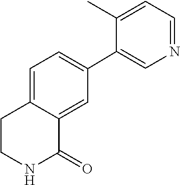 [SMILES: Cc1ccncc1-c1ccc2c(c1)C(=O)NCC2]

The titled compound 18 was prepared in a yield of 33.3% according to the procedure outlined for compound 5. Mass (m/z): 239.2 [M+H]⁺. ¹H NMR (400 MHz, DMSO-d₆) δ 8.45 (d, J=4.9 Hz, 1H), 8.37 (s, 1H), 8.05 (s, 1H), 7.80 (d, J=1.9 Hz, 1H), 7.52 (dd, J=7.7, 2.0 Hz, 1H), 7.43 (d, J=7.8 Hz, 1H), 7.36 (d, J=5.0 Hz, 1H), 3.43 (dd, J=6.6, 3.8 Hz, 2H), 2.97 (t, J=6.6 Hz, 2H), 2.26 (s, 3H).

### 2-fluoro-5-(4-methylpyridin-3-yl)phenol (19)

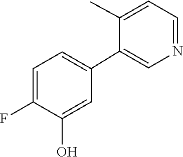

The titled compound 19 was prepared in a yield of 32.7% according to the procedure outlined for compound 5. Mass (m/z): 204.1 [M+H]⁺. ¹H NMR (400 MHz, Methanol-d₄) δ 8.65 (s, 2H), 7.95 (s, 1H), 7.26 (dd, J=11.0, 8.3 Hz, 1H), 7.03 (dd, J=8.2, 2.1 Hz, 1H), 6.89 (m, 1H), 2.57 (s, 3H).

### 3-fluoro-5-(4-methylpyridin-3-yl)phenol (20)

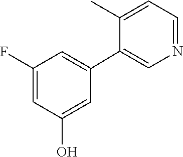

The titled compound 20 was prepared in a yield of 20.1% according to the procedure outlined for compound 5. Mass (m/z): 203.9[M+H]⁺. ¹H NMR (400 MHz, Methanol-d₄) δ 8.35 (d, J=5.1 Hz, 1H), 8.31 (d, J=25.8 Hz, 1H), 7.34 (d, J=5.1 Hz, 1H), 6.54 (qt, J=2.3, 1.9 Hz, 3H), 2.30 (s, 3H).

### 5-(4-methylpyridin-3-yl)-1H-indole (21)

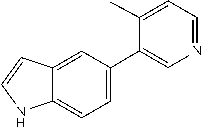 [SMILES: Cc1ccncc1-c1ccc2[nH]ccc2c1]

The titled compound 21 was prepared in a yield of 6.4% according to the procedure outlined for compound 3. Mass (m/z): 209.1[M+H]⁺. ¹H NMR (400 MHz, DMSO-d₆) δ 11.23 (s, 1H), 8.42 (s, 2H), 7.58-7.44 (m, 2H), 7.43-7.28 (m, 2H), 7.09 (d, J=8.3 Hz, 1H), 6.48 (s, 1H), 2.30 (s, 3H).

### 3-fluoro-5-(4-methylpyridin-3-yl)phenol (22)

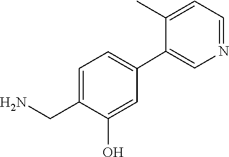 [SMILES: Cc1ccncc1-c1ccc(CN)c(O)c1]

The titled compound 22 was prepared in a yield of 10.4% according to the procedure outlined for compound 5. Mass (m/z): 215.0 [M+H]⁺. ¹H NMR (400 MHz, DMSO-d₆) δ 8.42 (d, J=5.0 Hz, 1H), 8.32 (s, 3H), 7.34 (t, J=6.8 Hz, 2H), 6.88 (d, J=1.6 Hz, 1H), 6.82 (dd, J=7.7, 1.6 Hz, 1H), 3.98 (s, 2H), 2.26 (s, 3H).

### 6-(4-methylpyridin-3-yl)-1H-indole (23)

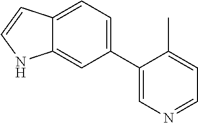 [SMILES: Cc1ccncc1-c1ccc2cc[nH]c2c1]

The titled compound 23 was prepared in a yield of 6.4% according to the procedure outlined for compound 3. Mass (m/z): 209.1 [M+H]⁺. ¹H NMR (400 MHz, DMSO-d₆) δ 11.20 (s, 1H), 8.40 (d, J=4.8 Hz, 2H), 7.62 (dd, J=8.2, 3.1 Hz, 1H), 7.46-7.24 (m, 3H), 7.00 (t, J=5.5 Hz, 1H), 6.53-6.39 (m, 1H), 2.29 (d, J=3.3 Hz, 3H).

### 5-(4-methylpyridin-3-yl)-1H-indazole (24)

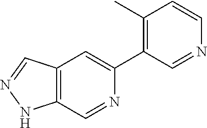 [SMILES: Cc1ccncc1-c1cc2cn[nH]c2cn1]

The titled compound 24 was prepared in a yield of 27% according to the procedure outlined for compound 5. Mass (m/z): 210.1 [M+H]⁺. ¹H NMR (400 MHz, DMSO-d₆) δ 13.19 (s, 1H), 8.46-8.39 (m, 2H), 8.12 (s, 1H), 7.76 (s, 1H), 7.63 (d, J=8.6 Hz, 1H), 7.40-7.30 (m, 2H), 2.27 (s, 3H).

### 5-(4-methylpyridin-3-yl)-1H-benzo[d]imidazole (25)

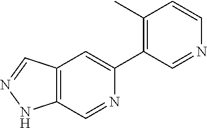 [SMILES: Cc1ccncc1-c1cc2cn[nH]c2cn1]

The titled compound 25 was prepared in a yield of 43% according to the procedure outlined for compound 5. Mass (m/z): 210.0 [M+H]⁺. ¹H NMR (400 MHz, DMSO-d₆) δ 12.56 (s, 1H), 8.46-8.38 (m, 2H), 8.28 (s, 1H), 7.82-7.46 (m, 2H), 7.37-7.29 m, 1H), 7.25-7.15 (m, 1H), 2.28 (d, J=2.4 Hz, 3H).

### 2-methyl-3-(4-methylpyridin-3-yl)phenol (26)

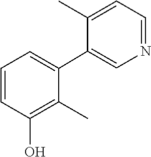

The titled compound 26 was prepared in a yield of 22.5% according to the procedure outlined for compound 5. Mass (m/z): 200.0 [M+H]⁺. ¹H NMR (400 MHz, DMSO-d₆) δ 9.50 (s, 1H), 8.42 (d, J=5.0 Hz, 1H), 8.21 (s, 1H), 7.32 (d, J=5.0 Hz, 1H), 7.07 (t, J=7.8 Hz, 1H), 6.86 (d, J=7.2 Hz, 1H), 6.55 (dd, J=7.6, 1.0 Hz, 1H), 2.03 (s, 3H), 1.80 (s, 3H).

### 2-chloro-3-(4methylpyridin-3-yl)phenol (27)

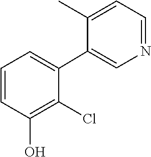

The titled compound 27 was prepared in a yield of 22.4% according to the procedure outlined for compound 5. Mass (m/z): 219.1 [M+H]⁺. ¹H NMR (400 MHz, DMSO-d₆) δ 8.45 (d, J=5.0 Hz, 1H), 8.25 (s, 1H), 7.34 (d, J=5.0 Hz, 11H), 7.19 (t, J=7.8 Hz, 11H), 7.01 (d, J=8.0 Hz, 11H), 6.67 (d, J=7.2 Hz, 1H), 2.08 (d, J=3.4 Hz, 3H).

### 2-methyl-4-(4-methylpyridin-3-yl)phenol (28)

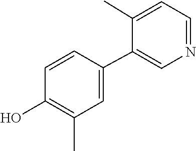

The titled compound 28 was prepared in a yield of 15% according to the procedure outlined for compound 5. Mass (m/z): 200.1 [M+H]⁺. ¹H NMR (400 MHz, DMSO-d₆) δ 9.48 (d, J=2.6 Hz, 1H), 8.38-8.27 (m, 2H), 7.27 (d, J=4.9 Hz, 1H), 7.08 (s, 1H), 7.01 (d, J=8.2 Hz, 1H), 6.86 (d, J=8.2 Hz, 1H), 2.25 (d, J=2.5 Hz, 3H), 2.16 (s, 3H).

### 2-ethyl-4-(4-methylpyridin-3-yl)phenol (29)

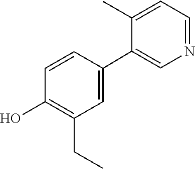 [SMILES: CCc1cc(-c2cnccc2C)ccc1O]

The titled compound 29 was prepared in a yield of 5.2% according to the procedure outlined for compound 5. Mass (m/z): 214.1 [M+H]⁺. ¹H NMR (400 MHz, DMSO-d₆) δ 9.46 (d, J=2.6 Hz, 1H), 8.37-8.27 (m, 2H), 7.27 (d, J=4.8 Hz, 1H), 7.08 (s, 1H), 7.02 (d, J=8.1 Hz, 1H), 6.86 (d, J=8.3 Hz, 1H), 2.58 (d, J=7.6 Hz, 2H), 2.26 (s, 3H), 1.15 (t, J=7.8 Hz, 3H).

### 2-methoxy-4-(4-methylpyridin-3-yl)phenol (30)

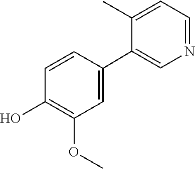 [SMILES: COc1cc(-c2cnccc2C)ccc1O]

The titled compound 30 was prepared in a yield of 7.7% according to the procedure outlined for compound 5. Mass (m/z): 216.1 [M+H]⁺. ¹H NMR (400 MHz, DMSO-d₆) δ 9.15 (d, J=2.7 Hz, 1H), 8.37 (d, J=7.0 Hz, 2H), 7.28 (d, J=4.9 Hz, 1H), 6.92 (s, 1H), 6.89-6.83 (m, 1H), 6.77 (d, J=8.2 Hz, 1H), 3.79 (d, J=2.4 Hz, 3H), 2.28 (s, 3H).

### 2-chloro-4-(4-methylpyridin-3-yl)phenol (31)

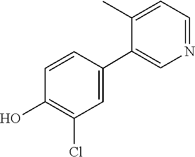

The titled compound 31 was prepared in a yield of 19.7% according to the procedure outlined for compound 5. Mass (m/z): 220.0 [M+H]⁺. ¹H NMR (400 MHz, DMSO-d₆) δ 10.39 (s, 1H), 8.46-8.27 (m, 2H), 7.37 (s, 1H), 7.30 (t, J=3.7 Hz, 1H), 7.17 (d, J=8.2 Hz, 1H), 7.06 (dd, J=8.3, 2.4 Hz, 1H), 2.25 (d, J=2.7 Hz, 3H).

### 2-methyl-5-(4-methylpyridin-3-yl)phenol (32)

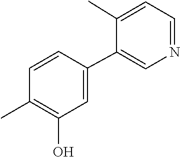

The titled compound 32 was prepared in a yield of 8.3% according to the procedure outlined for compound 5. Mass (m/z): 200.1 [M+H]⁺. ¹H NMR (400 MHz, DMSO-d₆) δ 9.45 (s, 1H), 8.43-8.35 (m, 1H), 8.31 (s, 1H), 7.30 (d, J=4.8 Hz, 1H), 7.15 (d, J=7.6 Hz, 1H), 6.79-6.66 (m, 2H), 2.25 (s, 3H), 2.17 (s, 3H).

### 5-(4-methylpyridin-3-yl)indoline (33)

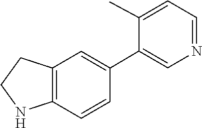 [SMILES: Cc1ccncc1-c1ccc2c(c1)CCN2]

The titled compound 33 was prepared in a yield of 41% according to the procedure outlined for compound 5. Mass (m/z): 211.1 [M+H]⁺. ¹H NMR (400 MHz, DMSO-d₆) δ 8.36-8.26 (m, 2H), 7.29-7.22 (m, 1H), 7.04 (s, 1H), 6.97-6.87 (m, 1H), 6.62-6.53 (m, 1H), 5.72 (s, 1H), 3.52-3.42 (m, 2H), 3.00-2.90 (m, 2H), 2.31-2.23 (s, 3H).

### 6-fluoro-5-(4-methylpyridin-3-yl)-1H-indole (34)

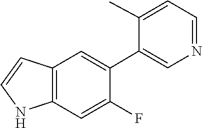 [SMILES: Cc1ccncc1-c1cc2cc[nH]c2cc1F]

The titled compound 34 was prepared in a yield of 47% according to the procedure outlined for compound 5. Mass (m/z): 227.1 [M+H]⁺. ¹H NMR (400 MHz, DMSO-d₆) δ 11.28 (s, 1H), 8.47-8.42 (m, 1H), 8.40-8.36 (m, 1H), 7.46 (dd, J=7.5, 2.5 Hz, 1H), 7.43-7.38 (m, 1H), 7.37-7.26 (m, 2H), 6.50-6.46 (m, 1H), 2.17 (s, 3H).

### 2,6-difluoro-3-(4-methylpyridin-3-yl)phenol (35)

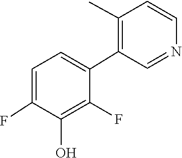 [SMILES: Cc1ccncc1-c1ccc(F)c(O)c1F]

The titled compound 35 was prepared in a yield of 33.9% according to the procedure outlined for compound 5. Mass (m/z): 221.9 [M+H]⁺. ¹H NMR (400 MHz, Methanol-d₄) δ 8.43 (d, J=5.2 Hz, 1H), 8.32 (s, 1H), 7.41 (d, J=5.2 Hz, 1H), 7.05 (m, 1H), 6.74 (m, 1H), 2.27 (s, 3H).

### 5-(4-methylpyridin-3-yl)-1H-benzol[d][1,2,3]triazole (36)

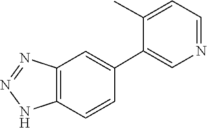 [SMILES: Cc1ccncc1-c1ccc2[nH]nnc2c1]

The titled compound 36 was prepared in a yield of 41% according to the procedure outlined for compound 5. Mass (m/z): 211.0 [M+H]⁺. ¹H NMR (400 MHz, Methanol-d₄) δ 8.58 (s, 1H), 8.56-8.50 (m, 1H), 7.89-7.79 (m, 3H), 7.35 (dd, J=8.5, 2.8 Hz, 1H), 2.38 (d, J=3.0 Hz, 3H).

### 7-fluoro-6-(4-methylpyridin-3-yl)indoline-2,3-dione (37)

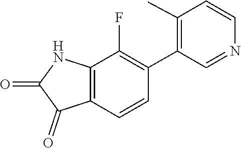 [SMILES: Cc1ccncc1-c1ccc2c(c1F)NC(=O)C2=O]

The titled compound 37 was prepared in a yield of 14% according to the procedure outlined for compound 5. Mass (m/z): 275.0 [M+H+H₂O]⁺. ¹H NMR (400 MHz, Methanol-d₄) δ 8.73 (s, 2H), 7.96 (s, 1H), 7.88-7.40 (m, 1H), 7.16-7.05 (m, 0.62H), 6.65-6.57 (m, 0.44H), 2.50 (d, J=5.2 Hz, 3H).

### 1-(2-hydroxy-4-(4-methylpyridin-3-yl)phenyl)ethan-1-one (38)

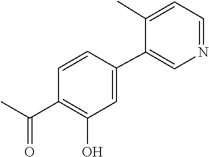 [SMILES: CC(=O)c1ccc(-c2cnccc2C)cc1O]

The titled compound 38 was prepared in a yield of 47% according to the procedure outlined for compound 5. Mass (m/z): 228.1 [M+H]⁺. ¹H NMR (400 MHz, DMSO-d₆) δ 12.06 (d, J=3.0 Hz, 1H), 8.55-8.31 (m, 2H), 7.97 (dd, J=8.5, 2.9 Hz, 1H), 7.42-7.27 (m, 1H), 6.99 (t, J=4.7 Hz, 2H), 2.80-2.62 (m, 3H), 2.28 (d, J=2.9 Hz, 3H).

### 3-fluoro-4-(4-methylpyridin-3-yl)benzene-1,2-diol (39)

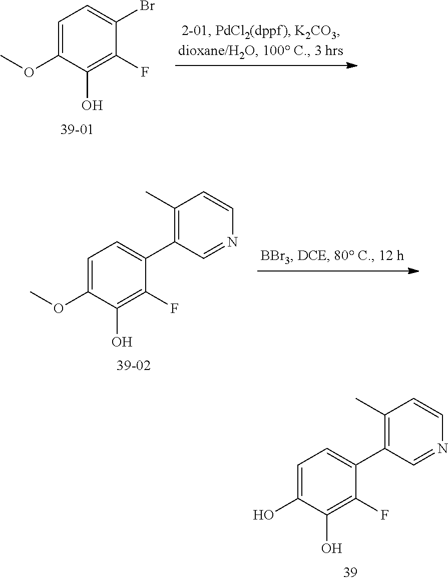 [SMILES: COc1ccc(Br)c(F)c1O ; COc1ccc(-c2cnccc2C)c(F)c1O ; Cc1ccncc1-c1ccc(O)c(O)c1F]

Step 1: Compound 39-02 was prepared according to the procedure outlined for compound 5. Purification by silica gel chromatography (MeOH in DCM=0% to 10%) to give 2-fluoro-6-methoxy-3-(4-methylpyridin-3-yl)phenol 39-02 (120 mg, 45%) as a white solid. Mass (m/z): 234.2 [M+H]⁺.

Step 2: To a solution of 2-fluoro-6-methoxy-3-(4-methylpyridin-3-yl)phenol 39-02 (120 mg, 0.51 mmol) in DCE (6 mL) was added BBr₃ (1.29 g, 5.14 mmol) dropwise under nitrogen protection. After the addition, the mixture was heated to 80° C. and stirred for 12 h. The reaction mixture was quenched with water (8 mL) then extracted with ethyl acetate (25 mL×2). The combined organic layer was washed with brine, dried over Na₂SO₄, and concentrated in vacuo to give a residue. The crude product was purified by silica gel chromatography (MeOH in DCM=0% to 10%) and re-purified by prep. HPLC to give the titled compound 39 (17.3 mg, yield: 15.3%) as a black oil. Mass (m/z): 220.2 [M+H]⁺. ¹H NMR (400 MHz, DMSO-d₆) δ 9.84 (s, 1H), 9.28 (s, 1H), 8.74-8.50 (m, 2H), 7.73 (d, J=5.6 Hz, 1H), 6.78-6.56 (m, 2H), 2.29 (s, 3H).

### 6-(4-methylpyridin-3-yl)benzo[d]oxazol-2(3H)-one (40)

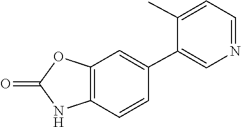 [SMILES: Cc1ccncc1-c1ccc2[nH]c(=O)oc2c1]

The titled compound 40 was prepared in a yield of 14.2% according to the procedure outlined for compound 5. Mass (m/z): 227.1 [M+H]⁺. ¹H NMR (400 MHz, DMSO-d₆) δ 11.76 (s, 1H), 8.42 (d, J=5.0 Hz, 1H), 8.36 (s, 1H), 7.39 (d, J=1.4 Hz, 1H), 7.33 (d, J=5.0 Hz, 1H), 7.22-7.13 (m, 2H), 2.27 (s, 3H).

### 5-(4-methylpyridin-3-yl)indolin-2-one (41)

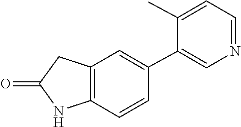 [SMILES: Cc1ccncc1-c1ccc2c(c1)CC(=O)N2]

The titled compound 41 was prepared in a yield of 11.3% according to the procedure outlined for compound 5. Mass (m/z): 224.9 [M+H]⁺. ¹H NMR (400 MHz, Methanol-d₄) δ 8.36 (d, J=5.1 Hz, 1H), 8.32 (s, 1H), 7.38 (d, J=5.1 Hz, 1H), 7.29 (s, 1H), 7.23 (dd, J=8.0, 1.2 Hz, 1H), 7.03 (d, J=8.0 Hz, 1H), 3.63 (s, 1H), 3.61 (s, 1H), 2.35 (s, 3H).

### 2-amino-5-(4-methylpyridin-3-yl)phenol (42)

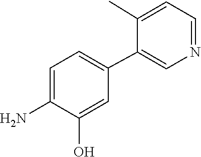

The titled compound 42 was prepared in a yield of 26.8% according to the procedure outlined for compound 5. Mass (m/z): 200.9 [M+H]⁺. ¹H NMR (400 MHz, Methanol-d₄) δ 8.27-8.21 (m, 2H), 7.28 (dd, J=4.4, 0.6 Hz, 11H), 6.80 (d, J=8.0 Hz, 11H), 6.66 (d, J=2.0 Hz, 11H), 6.61 (dd, J=8.0, 2.0 Hz, 1H), 2.31 (s, 3H).

### 2-amino-4-(4-methylpyridin-3-yl)phenol (43)

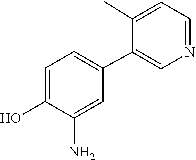

The titled compound 43 was prepared in a yield of 20.1% according to the procedure outlined for compound 5. Mass (m/z): 201.2 [M+H]⁺. ¹H NMR (400 MHz, Methanol-d₄) δ 8.33-8.14 (m, 2H), 7.28 (d, J=5.2 Hz, 1H), 6.73 (dd, J=24.4, 5.2 Hz, 2H), 6.52 (dd, J=8.0, 2.2 Hz, 1H), 2.30 (s, 3H).

### 5-(4-methylpyridin-3-yl)-1,3-dihydro-2H-benzo[d]imidazol-2-one (44)

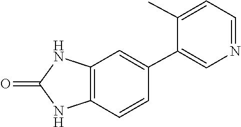 [SMILES: Cc1ccncc1-c1ccc2[nH]c(=O)[nH]c2c1]

The titled compound 44 was prepared in a yield of 20.2% according to the procedure outlined for compound 5. Mass (m/z): 225.9 [M+H]⁺. ¹H NMR (400 MHz, Methanol-d₄) δ 8.39-8.23 (m, 2H), 7.34 (d, J=5.2 Hz, 1H), 7.15-7.09 (m, 1H), 7.01-6.96 (m, 2H), 2.31 (s, 3H).

### 7-(4-methylpyridin-3-yl)-2H-benzo[b][1,4]oxazin-3(4H)-one (45)

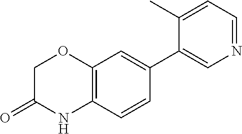 [SMILES: Cc1ccncc1-c1ccc2c(c1)OCC(=O)N2]

The titled compound 45 was prepared in a yield of 18.8% according to the procedure outlined for compound 5. Mass (m/z): 240.9 [M+H]⁺. ¹H NMR (400 MHz, DMSO-d₆) δ 10.84 (s, 1H), 8.41 (s, 1H), 8.34 (s, 1H), 7.32 (s, 1H), 7.01 (s, 1H), 6.98 (s, 2H), 4.63 (s, 2H), 2.27 (s, 3H).

### 6-(4-methylpyridin-3-yl)-3,4-dihydroquinazolin-2(1H)-one (46)

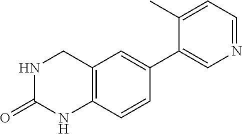 [SMILES: Cc1ccncc1-c1ccc2c(c1)CNC(=O)N2]

The titled compound 46 was prepared in a yield of 18.8% according to the procedure outlined for compound 5. Mass (m/z): 239.9 [M+H]⁺. ¹H NMR (400 MHz, DMSO-d₆) δ 9.15 (s, 1H), 8.38 (d, J=5.0 Hz, 1H), 8.33 (s, 1H), 7.30 (d, J=5.0 Hz, 1H), 7.14 (s, 2H), 6.86 (d, J=7.9 Hz, 2H), 4.36 (s, 2H), 2.27 (s, 3H).

### 4-(4-methylpyridin-3-yl)benzene-1,2-diol (47)

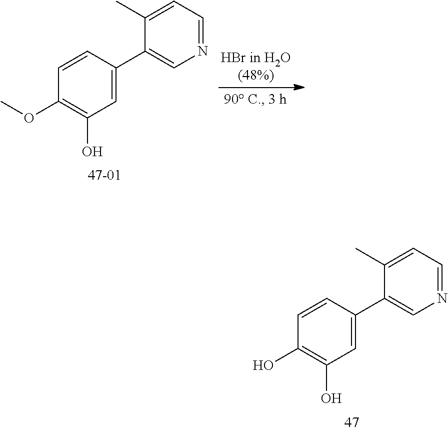 [SMILES: COc1ccc(-c2cnccc2C)cc1O ; Cc1ccncc1-c1ccc(O)c(O)c1]

A mixture of compound 47-01 (108 mg, 0.5 mmol) in HBr (48%, 4 mL) was stirred at 90° C. for 3 hours. The mixture concentrated in vacuo. Purification by silica gel chromatography gave the compound 47 (50 mg, yield: 49.7%) as a white solid. Mass (m/z): 202.1 [M+H]⁺. ¹H NMR (400 MHz, DMSO-d₆) δ 9.35 (s, 2H), 8.62 (d, J=9.2 Hz, 2H), 7.80 (d, J=5.5 Hz, 1H), 6.91-6.80 (m, 2H), 6.72 (d, J=8.2 Hz, 1H), 2.45 (s, 3H).

### 5-(4-methylpyridin-3-yl)-1H-pyrazolo[3,4-b]pyridine (48)

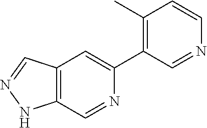 [SMILES: Cc1ccncc1-c1cc2cn[nH]c2cn1]

The titled compound 48 was prepared in a yield of 28% according to the procedure outlined for compound 5. Mass (m/z): 211.1 [M+H]⁺. ¹H NMR (400 MHz, DMSO-d₆) δ 13.79 (s, 1H), 8.55 (s, 1H), 8.51-8.43 (m, 2H), 8.30 (s, 1H), 8.20 (s, 1H), 7.42-7.36 (m, 1H), 2.29 (d, J=3.1 Hz, 3H).

### 5-(4-methylpyridin-3-yl)-1H-pyrazolo[3,4-c]pyridine (49)

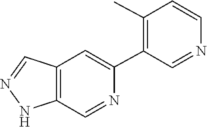 [SMILES: Cc1ccncc1-c1cc2cn[nH]c2cn1]

The titled compound 49 was prepared in a yield of 3% according to the procedure outlined for compound 5. Mass (m/z): 211.0 [M+H]⁺. ¹H NMR (400 MHz, Methanol-d₄) δ 9.13 (s, 1H), 8.79 (s, 1H), 8.62 (d, J=5.9 Hz, 1H), 8.25 (d, J=3.0 Hz, 1H), 8.05 (d, J=2.8 Hz, 1H), 7.89 (d, J=5.9 Hz, 1H), 2.60 (d, J=2.9 Hz, 3H).

### 5-(4-methylpyridin-3-yl)-1H-pyrazolo[4,3-b]pyridine (50)

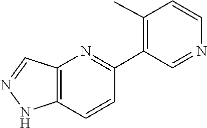 [SMILES: Cc1ccncc1-c1ccc2[nH]ncc2n1]

The titled compound 50 was prepared in a yield of 40% according to the procedure outlined for compound 5. Mass (m/z): 211.0 [M+H]⁺. ¹H NMR (400 MHz, DMSO-d₆) δ 13.43 (s, 1H), 8.59 (s, 1H), 8.51-8.45 (m, 11H), 8.36 (s, 11H), 8.13 (d, J=8.6 Hz, 11H), 7.59 (d, J=8.7 Hz, 11H), 7.40-7.34 (m, 1H), 2.38 (d, J=3.2 Hz, 3H).

### 6-(4-methylpyridin-3-yl)-2H-benzo[b][1,4]oxazin-3(4H)-one (51)

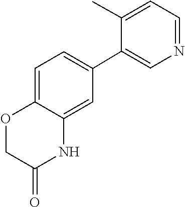 [SMILES: Cc1ccncc1-c1ccc2c(c1)NC(=O)CO2]

The titled compound 51 was prepared in a yield of 13.8% according to the procedure outlined for compound 5. Mass (m/z): 241.3 [M+H]⁺. ¹H NMR (400 MHz, Methanol-d₄) δ 8.35 (s, 1H), 8.30 (s, 1H), 7.36 (s, 1H), 7.06 (d, J=8.2 Hz, 1H), 6.96 (d, J=7.9 Hz, 1H), 6.88 (s, 1H), 4.63 (s, 2H), 2.33 (s, 3H).

### 5-(4-methylpyridin-3-yl)benzo[d]oxazol-2(3H)-one (52)

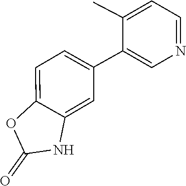 [SMILES: Cc1ccncc1-c1ccc2oc(=O)[nH]c2c1]

The titled compound 52 was prepared in a yield of 8.9% according to the procedure outlined for compound 5. Mass (m/z): 227.0 [M+H]⁺. ¹H NMR (400 MHz, Methanol-d₄) δ 8.38 (s, 1H), 8.33 (s, 1H), 7.43-7.21 (m, 2H), 7.08 (d, J=12.0 Hz, 2H), 2.32 (s, 3H).

### 7-(4-methylpyridin-3-yl)-3,4-dihydro-2H-benzo[b][1,4]oxazine (53)

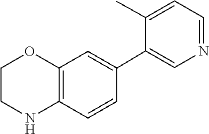 [SMILES: Cc1ccncc1-c1ccc2c(c1)OCCN2]

The titled compound 53 was prepared in a yield of 25.3% according to the procedure outlined for compound 5. Mass (m/z): 226.9 [M+H]⁺. ¹H NMR (400 MHz, DMSO-d₆) δ 8.34 (t, J=18.4 Hz, 1H), 7.26 (s, 1H), 6.66 (d, J=11.0 Hz, 1H), 5.97 (s, 1H), 4.13 (d, J=25.9 Hz, 2H), 3.31 (dd, J=13.4, 9.3 Hz, 2H), 2.27 (s, 3H).

### 6-(4-methylpyridin-3-yl)-3,4-dihydro-2H-benzo[b][1,4]oxazine (54)

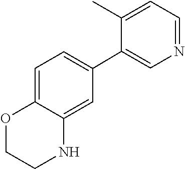 [SMILES: Cc1ccncc1-c1ccc2c(c1)NCCO2]

The titled compound 54 was prepared in a yield of 27.8% according to the procedure outlined for compound 5. Mass (m/z): 226.9 [M+H]⁺. ¹H NMR (400 MHz, DMSO-d₆) δ 8.35 (d, J=5.0 Hz, 1H), 8.28 (s, 1H), 7.27 (d, J=5.0 Hz, 1H), 6.72 (d, J=8.1 Hz, 1H), 6.54 (d, J=2.1 Hz, 1H), 6.45 (d, J=6.0 Hz, 1H), 5.87 (s, 1H), 4.16 (s, 2H), 3.31-3.30 (m, 2H), 2.25 (s, 3H).

### 6-(4-methylpyridin-3-yl)indolin-2-one (55)

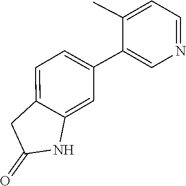 [SMILES: Cc1ccncc1-c1ccc2c(c1)NC(=O)C2]

The titled compound 55 was prepared in a yield of 28.0% according to the procedure outlined for compound 5. Mass (m/z): 225.1 [M+H]⁺. ¹H NMR (400 MHz, Methanol-d₄) δ 8.60 (s, 2H), 7.89 (s, 1H), 7.40 (d, J=7.2 Hz, 1H), 7.03 (d, J=7.3 Hz, 1H), 6.92 (s, 1H), 3.59 (s, 2H), 2.52 (s, 3H).

### 4′-methyl-[3,3′-bipyridin]-5-ol (56)

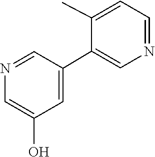

The titled compound 56 was prepared in a yield of 21.7% according to the procedure outlined for compound 5. Mass (m/z): 187.1 [M+H]⁺. ¹H NMR (400 MHz, DMSO-d₆) δ 10.13 (s, 1H), 8.46 (d, J=5.0 Hz, 1H), 8.38 (s, 1H), 8.19 (d, J=2.7 Hz, 1H), 8.06 (d, J=1.8 Hz, 1H), 7.36 (d, J=5.0 Hz, 1H), 7.18 (t, J=2.3 Hz, 1H), 2.27 (s, 3H).

### 1-methyl-6-(4-methylpyridin-3-yl)-1,3-dihydro-2H-benzo[d]imidazol-2-one (57)

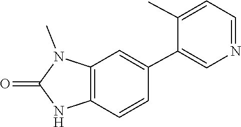 [SMILES: Cc1ccncc1-c1ccc2[nH]c(=O)n(C)c2c1]

The titled compound 57 was prepared in a yield of 46.3% according to the procedure outlined for compound 5. Mass (m/z): 240.2 [M+H]⁺. ¹H NMR (400 MHz, DMSO-d₆) δ 10.95 (s, 1H), 8.41 (d, J=5.0 Hz, 1H), 8.39 (s, 1H), 7.32 (d, J=5.0 Hz, 1H), 7.15 (d, J=1.5 Hz, 1H), 7.06 (d, J=8.0 Hz, 1H), 6.99 (dd, J=8.0, 1.6 Hz, 1H), 3.31 (s, 3H), 2.29 (s, 3H).

### 7-chloro-5-(4-methylpyridin-3-yl)-1H-indazole (58)

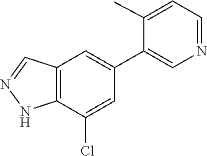 [SMILES: Cc1ccncc1-c1cc(Cl)c2[nH]ncc2c1]

The titled compound 58 was prepared in a yield of 51.2% according to the procedure outlined for compound 5. Mass (m/z): 244.2 [M+H]⁺. ¹H NMR (400 MHz, DMSO-d₆) δ 13.73 (s, 1H), 8.49-8.39 (m, 2H), 8.25 (d, J=1.4 Hz, 11H), 7.77 (d, J=1.2 Hz, 11H), 7.52 (d, J=1.3 Hz, 11H), 7.36 (d, J=5.0 Hz, 1H), 2.28 (s, 3H).

### 6-(4-methylpyridin-3-yl)-1,4-dihydro-2H-benzo[d][1,3]oxazin-2-one (59)

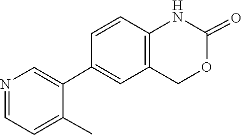 [SMILES: Cc1ccncc1-c1ccc2c(c1)COC(=O)N2]

The titled compound 59 was prepared in a yield of 51.2% according to the procedure outlined for compound 5. Mass (m/z): 241.1 [M+H]⁺. ¹H NMR (400 MHz, DMSO-d₆) δ 8.64 (s, 1H), 8.47 (d, J=5.0 Hz, 1H), 8.41 (s, 1H), 7.76 (d, J=7.8 Hz, 1H), 7.67-7.59 (m, 1H), 7.50 (dd, J=7.8, 1.5 Hz, 1H), 7.37 (d, J=5.0 Hz, 1H), 4.44 (s, 2H), 2.28 (s, 3H).

### 3-chloro-5-(4-methylpyridin-3-yl)phenol (60)

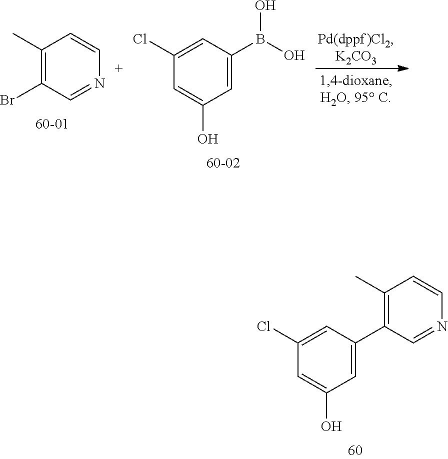

A mixture of compound 3-bromo-4-methylpyridine 60-01 (100 mg, 0.58 mmol), compound (3-chloro-5-hydroxyphenyl)boronic acid 60-02 (101 mg, 0.58 mmol), Pd(dppf)Cl₂ (45 mg, 0.05 mmol) and K₂CO₃ (243 mg, 1.74 mmol) in dioxane (5 mL)/H₂O (1 mL) was stirred under N₂ at 95° C. for 2 h. The reaction mixture was cooled to room temperature and diluted with water. The aqueous phase was extracted with ethyl acetate. The combined organic extracts were washed with brine and dried over Na₂SO₄. The mixture was concentrated and further purified by silica gel column chromatography to give compound 60 (17.8 mg, yield: 13.8%) as a white solid. Mass (m/z): 220.1 [M+H]⁺. 1H NMR (400 MHz, DMSO-d₆) δ 10.13 (s, 1H), 8.43 (d, J=4.8 Hz, 1H), 8.34 (s, 1H), 7.33 (d, J=4.8 Hz, 1H), 6.87-6.86 (m, 2H), 6.71 (s, 1H), 2.25 (s, 3H),

### 2-chloro-6-fluoro-4-(4-methylpyridin-3-yl)phenol (61)

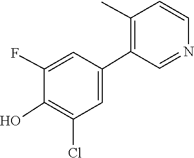 [SMILES: Cc1ccncc1-c1cc(F)c(O)c(Cl)c1]

The titled compound 61 was prepare in a yield of 16.4% according to the procedure outlined for compound 60. Mass (m/z): 238.1 [M+H]⁺. ¹H NMR (400 MHz, DMSO-d₆) δ 10.60 (s, 1H), 8.42 (d, J=4.8 Hz, 1H), 8.36 (s, 1H), 7.33-7.26 (m, 3H), 2.28 (s, 3H)

### 3-(3-(difluoromethyl)phenyl)-4-methylpyridine (62)

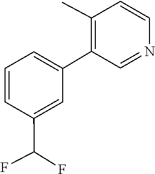 [SMILES: Cc1ccncc1-c1cccc(C(F)F)c1]

The titled compound 62 was prepared in a yield of 25.5% according to the procedure outlined for compound 60. Mass (m/z): 220.1 [M+H]⁺. ¹H NMR (400 MHz, DMSO-d₆) δ 8.47 (d, J=5.0 Hz, 1H), 8.40 (s, 1H), 7.68-7.57 (m, 4H), 7.37 (d, J=5.0 Hz, 1H), 7.10 (t, J=55.8 Hz, 1H), 2.27 (s, 3H).

### 3-(4-methylpyridin-3-yl)-5-(trifluoromethyl)phenol (63)

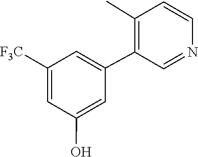 [SMILES: Cc1ccncc1-c1cc(O)cc(C(F)(F)F)c1]

The titled compound 63 was prepared in a yield of 25.5% according to the procedure outlined for compound 60. Mass (m/z): 254.2 [M+H]⁺. ¹H NMR (400 MHz, DMSO-d₆) δ 10.36 (s, 1H), 8.46 (d, J=5.0 Hz, 1H), 8.38 (s, 1H), 7.35 (d, J=5.0 Hz, 1H), 7.16-7.08 (m, 2H), 7.04 (m, 1H), 2.26 (s, 3H).

### 5-(4-methylpyridin-3-yl)-1H-benzo[d]imidazol-2-amine (64)

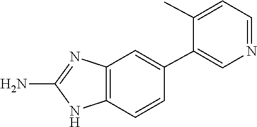 [SMILES: Cc1ccncc1-c1ccc2[nH]c(N)nc2c1]

The titled compound 64 was prepared in a yield of 12.6% according to the procedure outlined for compound 5. Mass (m/z): 225.0 [M+H]⁺. ¹H NMR (400 MHz, Methanol-d4) δ 8.49 (d, J=4.6 Hz, 1H), 7.51 (d, J=5.1 Hz, 11H), 7.43 (d, J=8.0 Hz, 11H), 7.30 (d, J=1.3 Hz, 11H), 7.11 (dd, J=8.0, 1.5 Hz, 1H), 2.51 (s, 3H).

### 5-(4-methylpyridin-3-yl)-1H-indazol-3-amine (65)

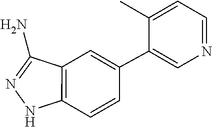 [SMILES: Cc1ccncc1-c1ccc2[nH]nc(N)c2c1]

The titled compound 65 was prepared in a yield of 46.8% according to the procedure outlined for compound 5. Mass (m/z): 225.1 [M+H]⁺. ¹H NMR (400 MHz, DMSO-d₆) δ 11.50 (s, 1H), 8.41-8.39 (m, 2H), 7.71 (s, 1H), 7.33-7.30 (m, 2H), 7.25 (dd, J=8.5, 1.5 Hz, 1H), 5.42 (s, 2H), 2.29 (s, 3H).

### 6-(6-chloro-4-methylpyridin-3-yl)benzo[d]oxazol-2(3H)-one (66)

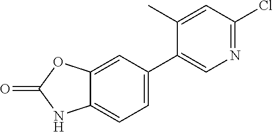 [SMILES: Cc1cc(Cl)ncc1-c1ccc2[nH]c(=O)oc2c1]

The titled compound 66 was prepared in a yield of 1.1% according to the procedure outlined for compound 5. Mass (m/z): 261.0 [M+H]⁺. ¹H NMR (400 MHz, DMSO-d₆) δ 11.79 (s, 1H), 8.21 (s, 1H), 7.52 (s, 1H), 7.42 (s, 1H), 7.19 (t, J=6.4 Hz, 2H), 2.28 (s, 3H).

### 6-(4,6-dimethylpyridin-3-yl)benzo[d]oxazol-2(3H)-one (67)

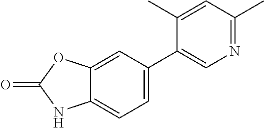 [SMILES: Cc1cc(C)c(-c2ccc3[nH]c(=O)oc3c2)cn1]

The titled compound 67 was prepared in a yield of 15.6% according to the procedure outlined for compound 60. Mass (m/z): 241.0 [M+H]⁺. ¹H NMR (400 MHz, DMSO-d₆) δ 11.90 (s, 1H), 8.55 (s, 1H), 7.70 (s, 1H), 7.47 (s, 1H), 7.23 (d, J=2.1 Hz, 2H), 2.63 (s, 3H), 2.41 (s, 3H).

### 3-fluoro-5-(4-methylpyridin-3-yl)-1H-indazole (68)

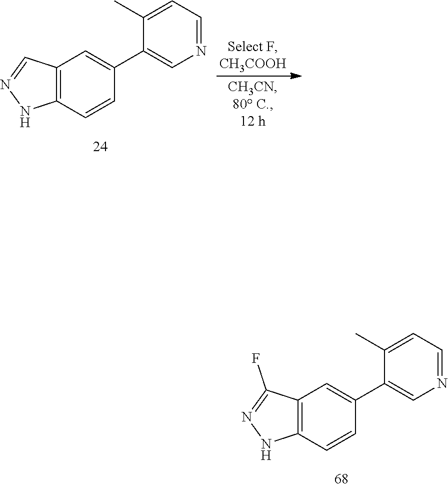 [SMILES: Cc1ccncc1-c1ccc2[nH]ncc2c1 ; Cc1ccncc1-c1ccc2[nH]nc(F)c2c1]

To a solution of 5-(4-methylpyridin-3-yl)-1H-indazole 24 (40 mg, 0.19 mmol) in CH₃CN (10 mL) and acetic acid (4 mL) was added Selectfluor (135 mg, 0.382 mmol) at rt (room temperature). The reaction mixture was stirred at 80° C. for 12 h. The reaction was cooled to rt and quenched with H₂O (20 mL). The pH of the resulting solution was adjusted to 7˜8 and the aqueous was extracted with ethyl acetate. The combined organic layers were washed with brine, dried over MgSO₄, filtered, and concentrated in vacuo. The crude product was purified by Prep-HPLC to afford compound 68 (6.5 mg, yield: 14.96%) as a white solid. Mass (m/z): 228.1 [M+H]⁺. ¹H NMR (400 MHz, DMSO-d₆) δ 8.41-8.28 (m, 2H), 7.57 (d, J=1.7 Hz, 11H), 7.38 (dd, J=8.0, 1.7 Hz, 11H), 7.22 (d, J=8.0 Hz, 11H), 7.04-6.93 (m, 2H).

### 1-(4-(4-methylpyridin-3-yl)phenyl)guanidine (69)

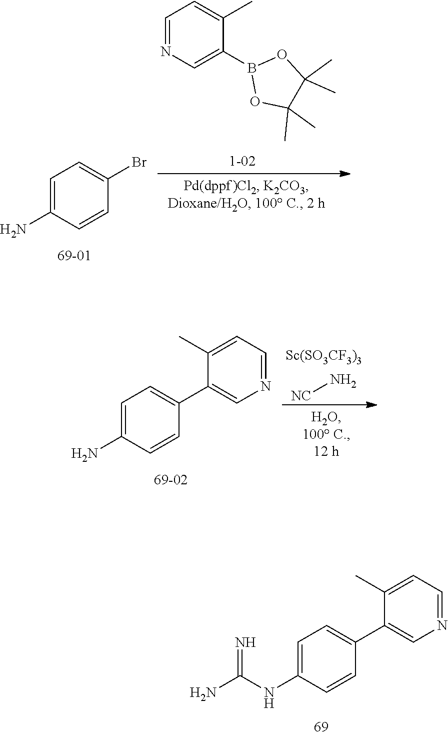

Step 1: 4-(4-Methylpyridin-3-yl) aniline 69-02 was prepared in a yield of 78% (250 mg, 1.36 mmol) from 4-bromoaniline 69-01 (300 mg, 1.74 mmol) according to the procedure outlined for compound 5. Mass (m/z): 185.1 [M+H]⁺.

Step 2: To a solution of 4-(4-methylpyridin-3-yl) aniline 69-02 (100 mg, 0.54 mmol) and Sc(SO₃CF₃)₃ (40 mg, 0.08 mmol) in water (4 mL) was added cyanamide (27 mg, 0.65 mmol) at rt. The reaction was stirred at 100° C. for 12 h. The reaction mixture was cooled to rt and extracted with EA. The combined organic layers were washed with brine, dried over MgSO₄, filtered, and concentrated in vacuo. The crude product was purified by prep-HPLC to afford compound 69 (4.5 mg, yield: 3.66%) as a white solid. Mass (m/z): 227.1 [M+H]⁺. ¹H NMR (400 MHz, Methanol-d₄) δ 8.70-8.61 (m, 2H), 7.92 (d, J=5.8 Hz, 1H), 7.60-7.52 (m, 2H), 7.50-7.43 (m, 2H), 2.56 (s, 3H).

### 1-methyl-6-(4-methylpyridin-3-yl)-1,3-dihydrobenzo[c][1,2,5]thiadiazole 2,2-dioxide (70)

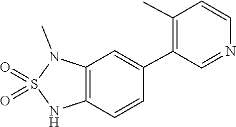 [SMILES: Cc1ccncc1-c1ccc2c(c1)N(C)S(=O)(=O)N2]

The titled compound 70 was prepared in a yield of 16.5% according to the procedure outlined for compound 5. Mass (m/z): 276.1 [M+H]⁺. ¹H NMR (400 MHz, DMSO-d₆) δ 8.63-8.54 (m, 2H), 7.68 (d, J=5.5 Hz, 1H), 7.08 (s, 1H), 6.99 (s, 2H), 3.21 (s, 3H), 2.42 (s, 3H).

### 6-(4-methylpyridin-3-yl)quinazolin-2-amine (71)

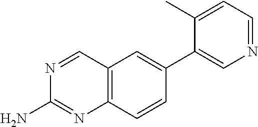 [SMILES: Cc1ccncc1-c1ccc2nc(N)ncc2c1]

The titled compound 71 was prepared in a yield of 15.5% according to the procedure outlined for compound 5. Mass (m/z): 237.1 [M+H]⁺. ¹H NMR (400 MHz, DMSO-d₆) δ 9.16 (s, 1H), 8.45 (t, J=4.0 Hz, 2H), 7.84 (d, J=2.0 Hz, 11H), 7.74 (dd, J=8.7, 2.0 Hz, 11H), 7.50 (d, J=8.0 Hz, 11H), 7.36 (d, J=4.0 Hz, 1H), 6.96 (s, 2H), 2.31 (s, 3H).

### 7-(4-methylpyridin-3-yl)-3,4-dihydroquinazolin-2(1H)-one (72)

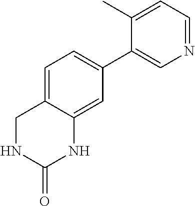 [SMILES: Cc1ccncc1-c1ccc2c(c1)NC(=O)NC2]

The titled compound 72 was prepared in a yield of 12.6% according to the procedure outlined for compound 5. Mass (m/z): 240.1 [M+H]⁺. ¹H NMR (400 MHz, DMSO-d₆) δ 9.08 (s, 1H), 8.42 (d, J=5.0 Hz, 1H), 8.32 (s, 11H), 7.33 (d, J=5.0 Hz, 11H), 7.18 (d, J=7.7 Hz, 11H), 6.87 (dd, J=7.6, 1.6 Hz, 2H), 6.74 (d, J=1.5 Hz, 1H), 4.37 (s, 2H), 2.25 (s, 3H).

### N-(5-(4-methylpyridin-3-yl)-1H-benzo[d]imidazol-2-yl)acetamide (73)

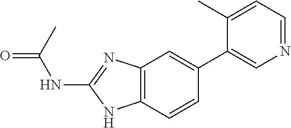 [SMILES: CC(=O)Nc1nc2cc(-c3cnccc3C)ccc2[nH]1]

The titled compound 73 was prepared in a yield of 20.0% according to the procedure outlined for compound 5. Mass (m/z): 267.0 [M+H]⁺. ¹H NMR (400 MHz, DMSO-d₆) δ 12.09 (d, J=10.0 Hz, 1H), 11.56 (s, 1H), 8.39 (dd, J=9.6, 4.0 Hz, 2H), 7.64-7.28 (m, 3H), 7.20-6.94 (m, 1H), 2.28 (s, 3H), 2.18 (s, 3H).

### 4-fluoro-5-(4-methylpyridin-3-yl)-1H-indazole (74)

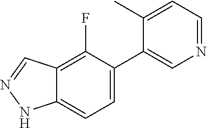 [SMILES: Cc1ccncc1-c1ccc2[nH]ncc2c1F]

The titled compound 74 was prepared in a yield of 17.6% according to the procedure outlined for compound 5. Mass (m/z): 228.1 [M+H]⁺. ¹H NMR (400 MHz, DMSO-d₆) δ 13.60 (s, 1H), 8.68 (td, J=5.7, 2.7 Hz, 2H), 8.30 (d, J=1.0 Hz, 1H), 7.76 (q, J=5.2 Hz, 1H), 7.54 (dd, J=8.5, 1.0 Hz, 1H), 7.36 (dd, J=8.5, 6.8 Hz, 1H), 2.32 (s, 3H).

### 6-(4-methylpyridin-3-yl)benzo[d]oxazole-2(3H)-thione (75)

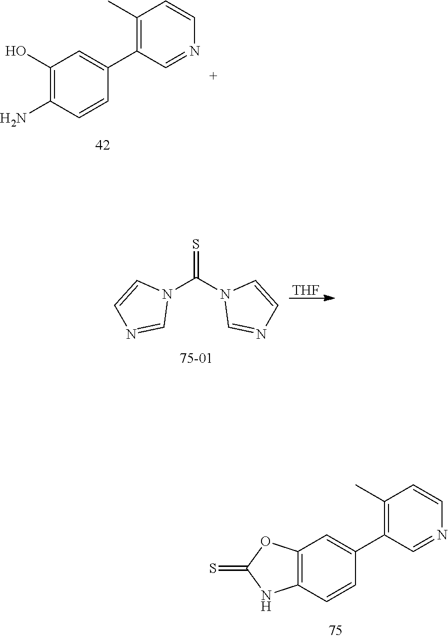 [SMILES: Cc1ccncc1-c1ccc(N)c(O)c1 ; S=C(n1ccnc1)n1ccnc1 ; "THF" ; Cc1ccncc1-c1ccc2[nH]c(=S)oc2c1]

A mixture of compound 2-amino-5-(4-methylpyridin-3-yl)phenol 42 (60 mg, 0.3 mmol) and compound di(1H-imidazol-1-yl)methanethione 75-01 (53.4 mg, 0.3 mmol) in THE (2 mL) was stirred at 80° C. for 2 h. The reaction mixture was cooled to rt and diluted with water. The aqueous phase was extracted with ethyl acetate. The combined organic extracts were washed with brine and dried over Na₂SO₄. The solvent was removed under vacuum and purified by prep-TLC to give compound 75 (30 mg, yield: 41.1%). Mass (m/z): 243.2 [M+H]⁺. ¹H NMR (400 MHz, DMSO-d₆) δ 14.00 (s, 1H), 8.45 (d, J=5.0 Hz, 1H), 8.40 (s, 1H), 7.64 (t, J=1.0 Hz, 1H), 7.39-7.31 (m, 3H), 2.28 (s, 3H).

### 5-(4-methylpyridin-3-yl)indoline-2,3-dione (76)

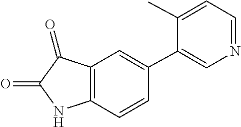 [SMILES: Cc1ccncc1-c1ccc2c(c1)C(=O)C(=O)N2]

The titled compound 76 was prepared in a yield of 38.1% according to the procedure outlined for compound 5. Mass (m/z): 239.2 [M+H]⁺. ¹H NMR (400 MHz, DMSO-d₆) δ 11.16 (s, 1H), 8.43 (d, J=5.0 Hz, 1H), 8.36 (s, 1H), 7.63 (dd, J=8.1, 1.9 Hz, 1H), 7.54 (d, J=1.8 Hz, 1H), 7.34 (d, J=5.0 Hz, 1H), 7.02 (d, J=8.1 Hz, 1H), 2.27 (s, 3H).

### 6-(5-aminopyridin-3-yl)benzo[d]oxazol-2(3H)-one (77)

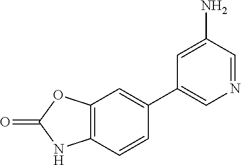 [SMILES: Nc1cncc(-c2ccc3[nH]c(=O)oc3c2)c1]

The titled compound 77 was prepared in a yield of 2.6% according to the procedure outlined for compound 5. Mass (m/z): 228.1 [M+H]⁺. ¹H NMR (400 MHz, DMSO-d₆) δ 11.89 (s, 1H), 8.26 (d, J=1.8 Hz, 1H), 7.95 (d, J=2.4 Hz, 1H), 7.69 (d, J=1.7 Hz, 1H), 7.65 (s, 1H), 7.48 (dd, J=8.1, 1.8 Hz, 1H), 7.24 (d, J=8.1 Hz, 1H), 6.24 (s, 2H).

### 6-(4-methylpyrimidin-5-yl)benzo[d]oxazol-2(3H)-one (78)

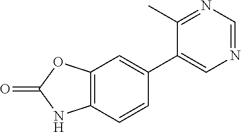 [SMILES: Cc1ncncc1-c1ccc2[nH]c(=O)oc2c1]

The titled compound 78 was prepare in a yield of 89% according to the procedure outlined for compound 5. Mass (m/z): 228.0 [M+H]⁺. ¹H NMR (400 MHz, DMSO-d₆) δ 9.03 (s, 1H), 8.59 (s, 1H), 7.49 (d, J=1.4 Hz, 1H), 7.31-7.16 (m, 2H), 2.47 (s, 3H).

### 6-(4-methylpyridin-3-yl)-3,4-dihydroquinazolin-2(1H)-imine (79)

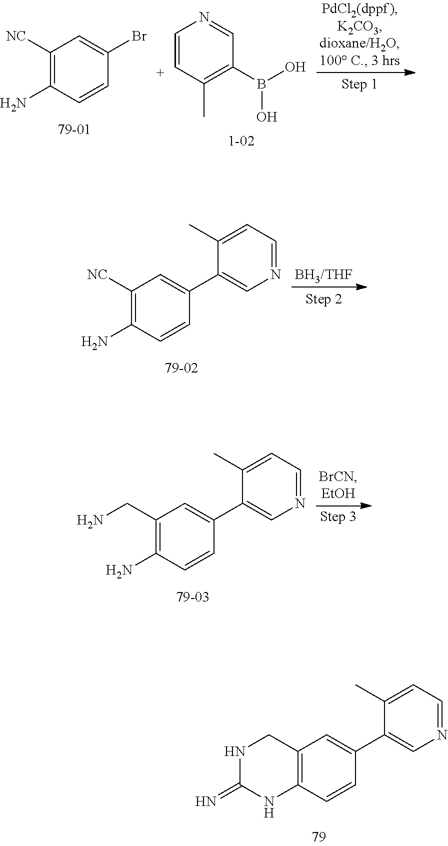

Step 1: 2-Amino-5-(4-methylpyridin-3-yl)benzonitrile 79-02 was prepared in a yield of 48.0% according to the procedure outlined for compound 5. Mass (m/z): 210.3 [M+H]⁺.

Step 2: To a solution of 2-amino-5-(4-methylpyridin-3-yl)benzonitrile 79-02 (300 mg, 1.43 mmol) in THF (3 mL) was added BH₃ (6 mL, 6 mmol, 1 M in THF) slowly. After the addition, the reaction mixture was stirred at 65° C. for 3 h under nitrogen protection. Then the reaction mixture was quenched by the addition of the MeOH (3 mL) dropwise at 0° C. and then stirred at 50° C. for 5 h. The solvent was removed under vacuum to give a residue. The residue was dissolved with DCM (10 mL) and water (10 mL), the organic layer was separated and the aqueous layer was extracted with DCM (20 mL×3). The combined organic phase was washed with brine, dried over Na₂SO₄ and concentrated in vacuo to give a crude product, which was purified by silica gel chromatography (MeOH in DCM=0% to 20%) to give 2-(aminomethyl)-4-(4-methylpyridin-3-yl) aniline 79-03 (100 mg, 32.7%) as a light brown slurry. Mass (m/z): 214.3 [M+H]⁺.

Step 3: A mixture of 2-(aminomethyl)-4-(4-methylpyridin-3-yl) aniline 79-03 (200 mg, 0.94 mmol) and BrCN (149 mg, 1.41 mmol) in EtOH (15 mL) was heated to 60° C. and stirred for 3 h under nitrogen protection. The reaction mixture was quenched with water (6 mL) and extracted with DCM (20 mL×2). The combined organic layer was washed with brine, dried over Na₂SO₄, and concentrated in vacuo to give a residue, which was purified by silica gel chromatography (MeOH in DCM=0% to 10%) and re-purified by prep-HPLC to give the titled compound 79 (18 mg, yield: 8.9%) as a light orange solid. Mass (m/z): 239.1 [M+H]⁺. ¹H NMR (400 MHz, Methanol-d₄) δ 8.35 (d, J=5.1 Hz, 1H), 8.30 (s, 1H), 7.36 (d, J=5.1 Hz, 1H), 7.23 (dd, J=8.2, 2.0 Hz, 1H), 7.13 (s, 1H), 7.02 (d, J=8.2 Hz, 1H), 4.58 (s, 2H), 2.33 (s, 3H).

### 7-(4-methylpyridin-3-yl)-1,5-dihydrobenzo[e][1,4]oxazepin-2(3H)-one (80)

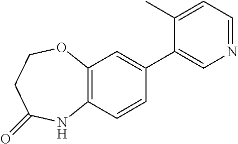 [SMILES: Cc1ccncc1-c1ccc2c(c1)OCCC(=O)N2]

The titled compound 80 was prepared in a yield of 29.1% according to the procedure outlined for compound 5. Mass (m/z): 255.1 [M+H]⁺. ¹H NMR (400 MHz, Methanol-d₄) δ 8.47-8.10 (m, 2H), 7.36 (d, J=5.1 Hz, 1H), 7.28 (dd, J=8.3, 2.0 Hz, 1H), 7.23-7.12 (m, 2H), 4.81 (s, 2H), 4.55 (s, 2H), 2.33 (s, 3H).

### 3,3-difluoro-5-(4-methylpyridin-3-yl)indolin-2-one (81)

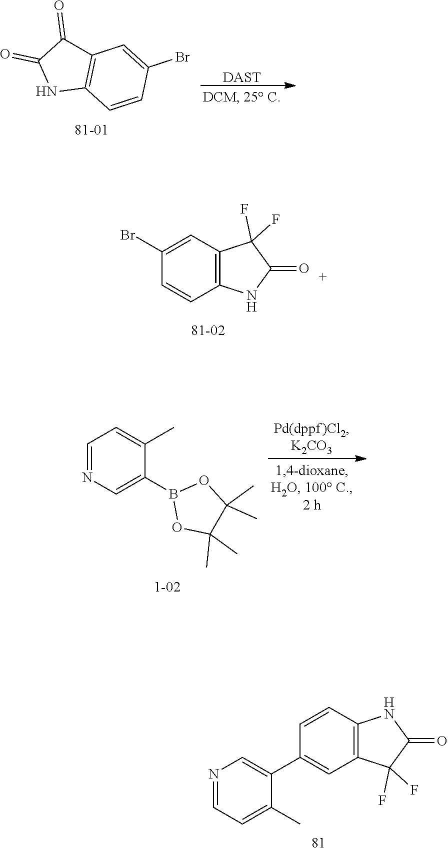

Step 1: To a solution of 5-bromoindoline-2,3-dione 81-01 (1.00 eq, 500 mg, 2.21 mmol) in DCM (10 mL) was added DAST (1.0 eq, 357 mg, 2.21 mmol). The mixture was stirred at RT for 2 h. The reaction was concentrated, and the residue was dissolved in ethyl acetate, washed with water, dried over Na₂SO₄ and concentrated to give the crude product. The crude product was purified by flash column chromatography to give the compound 81-02 (450 mg, yield: 75.5%). Step 2: The titled compound 81 was prepared in a yield of 36.2% according to the procedure outlined for compound 5. Mass (m/z): 243.1 [M+H]⁺. ¹H NMR (400 MHz, DMSO-d₆) δ 11.33 (s, 11H), 8.44 (d, J=5.0 Hz, 11H), 8.38 (s, 11H), 7.76 (d, J=2.0 Hz, 11H), 7.55 (dd, J=8.2, 1.8 Hz, 11H), 7.34 (d, J=5.0 Hz, 11H), 7.10 (dd, J=8.1, 1.8 Hz, 1H), 2.28 (s, 3H).

### 6-(5-methyl-2-oxo-1,2-dihydropyridin-4-yl)benzo[d]oxazol-2(3H)-one (82)

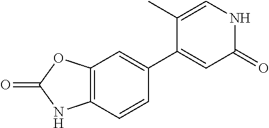 [SMILES: Cc1c[nH]c(=O)cc1-c1ccc2[nH]c(=O)oc2c1]

The titled compound 82 was prepared in a yield of 6.6% according to the procedure outlined for compound 60. Mass (m/z): 243.1 [M+H]⁺. ¹H NMR (400 MHz, DMSO-d₆) δ 11.77 (s, 1H), 11.49 (s, 1H), 7.34 (d, J=1.4 Hz, 1H), 7.27 (s, 1H), 7.16-7.08 (m, 2H), 6.16 (s, 1H), 1.90 (s, 3H).

### 6-(4-ethylpyridin-3-yl)benzo[d]oxazol-2(3H)-one (83)

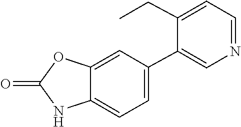 [SMILES: CCc1ccncc1-c1ccc2[nH]c(=O)oc2c1]

The titled compound 83 was prepared in a yield of 11.1% according to the procedure outlined for compound 60. Mass (m/z): 241.1 [M+H]⁺. ¹H NMR (400 MHz, DMSO-d₆) δ 11.86 (s, 1H), 8.68 (dd, J=5.8, 2.3 Hz, 1H), 8.58 (d, J=2.5 Hz, 11H), 7.76 (d, J=5.5 Hz, 11H), 7.43 (d, J=1.5 Hz, 11H), 7.27-7.15 (m, 2H), 2.72 (q, J=7.5 Hz, 2H), 1.10 (t, J=7.5 Hz, 3H).

### 6-(pyridin-3-yl)benzo[d]oxazol-2(3H)-one (84)

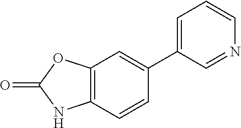 [SMILES: O=c1[nH]c2ccc(-c3cccnc3)cc2o1]

The titled compound 84 was prepared in a yield of 23.6% according to the procedure outlined for compound 5. Mass (m/z): 213.2 [M+H]^{+ 1}H NMR (400 MHz, DMSO-d₆) δ 11.84 (s, 1H), 9.00 (d, J=2.3 Hz, 1H), 8.65 (dd, J=5.0, 1.5 Hz, 1H), 8.34 (dt, J=8.2, 1.9 Hz, 1H), 7.78 (d, J=1.7 Hz, 1H), 7.68 (dd, J=8.1, 5.1 Hz, 1H), 7.57 (dd, J=8.1, 1.7 Hz, 1H), 7.23 (d, J=8.1 Hz, 1H).

### 8-(4-methylpyridin-3-yl)-2,3-dihydrobenzo[b][1,4]oxazepin-4(5H)-one (85)

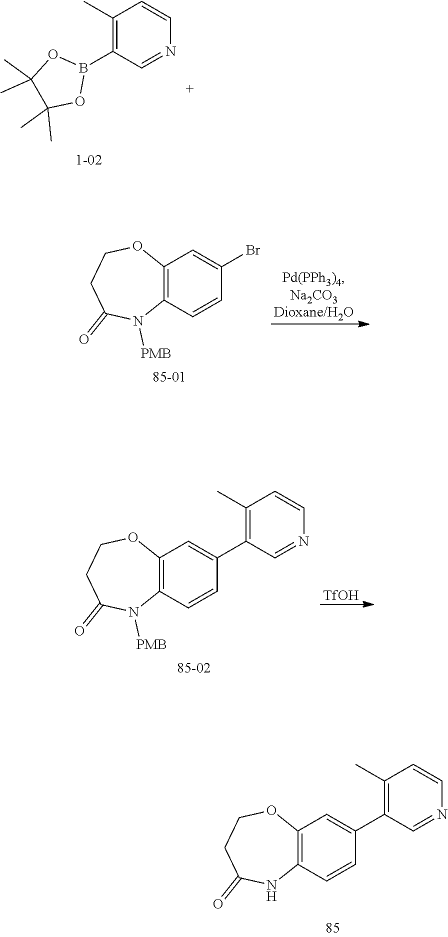

Step 1: 5-(4-methoxybenzyl)-8-(4-methylpyridin-3-yl)-2,3-dihydrobenzo[b][1,4]oxazepin-4(5H)-one 85-02 was prepared in a yield of 77.9% according to the procedure outlined for compound 3.

Step 2: A mixture of 5-(4-methoxybenzyl)-8-(4-methylpyridin-3-yl)-2,3-dihydrobenzo[b][1,4]oxazepin-4(5H)-one 85-02 (85 mg, 0.23 mmol) and TfOH (2 mL) was stirred at 70° C. for 1 h. The reaction mixture was cooled to rt and diluted with water. The aqueous phase was extracted with EA. The combined organic extracts were washed with brine and dried over Na₂SO₄. The solvent was removed under vacuum and purified by prep-HPLC to give compound 85 (45 mg, yield: 77.6%) as a white solid. Mass (m/z): 255.2 [M+H]⁺. ¹H NMR (400 MHz, DMSO-d₆) δ 9.92 (s, 1H), 8.59-8.50 (m, 2H), 7.62 (d, J=5.4 Hz, 1H), 7.19 (d, J=8.9 Hz, 1H), 7.08-6.93 (m, 2H), 4.39 (dd, J=6.1, 5.1 Hz, 2H), 2.78 (t, J=5.6 Hz, 2H), 2.40 (s, 3H).

### 6-(4-methylpyridin-3-yl)benzo[d]thiazol-2(3H)-one (86)

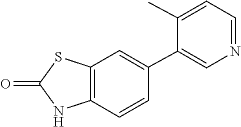 [SMILES: Cc1ccncc1-c1ccc2[nH]c(=O)sc2c1]

The titled compound 86 was prepared in a yield of 49.8% according to the procedure outlined for compound 5. Mass (m/z): 243.1 [M+H]⁺. ¹H NMR (400 MHz, DMSO-d₆) δ 12.11 (s, 1H), 8.63 (d, J=1.6 Hz, 2H), 7.73 (d, J=1.7 Hz, 2H), 7.37 (d, J=1.8 Hz, 1H), 7.26 (s, 1H), 2.43 (s, 3H).

### 6-(5-(phenylamino)pyridin-3-yl)benzo[d]oxazol-2(3H)-one (87)

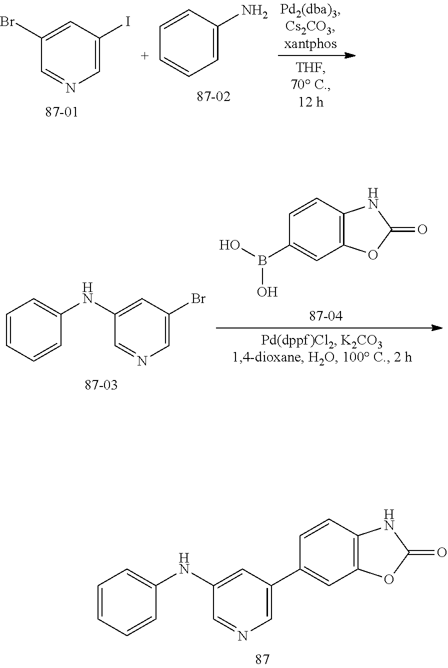 [SMILES: Brc1cncc(I)c1 ; NC1=CC=[C+]C=C1 ; O=c1[nH]c2ccc(B(O)O)cc2o1 ; Brc1cncc(Nc2ccccc2)c1 ; O=c1[nH]c2ccc(-c3cncc(Nc4ccccc4)c3)cc2o1]

Step 1: A mixture of 3-bromo-5-iodopyridine 87-01 (5 g, 17.6 mmol), aniline 87-02 (1.64 g, 17.6 mmol), Pd₂(dba)₃ (732 mg, 0.8 mmol), Cs₂CO₃ (17.2 g, 53 mmol) and Xantphos (462 mg, 0.8 mmol) was stirred under N₂ at 70° C. for 12 h. The reaction mixture was cooled to rt and diluted with water. The aqueous phase was extracted with EA. The combined organic extracts were washed with brine and dried over Na₂SO₄. The solvent was removed under vacuum to give crude 5-bromo-N-phenylpyridin-3-amine 87-03 which was used directly to the next step.

Step 2: Compound 87 was prepared in a yield of 38.2% according to the procedure outlined for compound 5. Mass (m/z): 304.1 [M+H]⁺. ¹H NMR (400 MHz, DMSO-d₆) δ 11.85 (s, 1H), 8.82 (s, 1H), 8.41 (d, J=1.9 Hz, 11H), 8.33 (d, J=2.5 Hz, 11H), 7.84 (t, J=2.2 Hz, 11H), 7.72 (d, J=1.7 Hz, 1H), 7.50 (dd, J=8.1, 1.7 Hz, 1H), 7.35 (dd, J=8.5, 7.3 Hz, 2H), 7.26-7.19 (m, 3H), 7.03-6.96 (m, 1H).

### 5-(4-methylpyridin-3-yl)-1H-indole-2-carbonitrile (88)

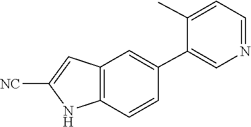 [SMILES: Cc1ccncc1-c1ccc2[nH]c(C#N)cc2c1]

The titled compound 88 was prepared in a yield of 5.52% according to the procedure outlined for compound 5. Mass (m/z): 234.1 [M+H]⁺. ¹H NMR (400 MHz, Methanol-d₄) δ 8.36 (d, J=5.6 Hz, 2H), 7.63 (d, J=0.7 Hz, 1H), 7.54 (d, J=8.6 Hz, 1H), 7.43-7.27 (m, 2H), 7.26 (s, 1H), 2.33 (s, 3H).

### 5-(4-methylpyridin-3-yl)-1H-benzo[d]imidazole-2-carbonitrile (89)

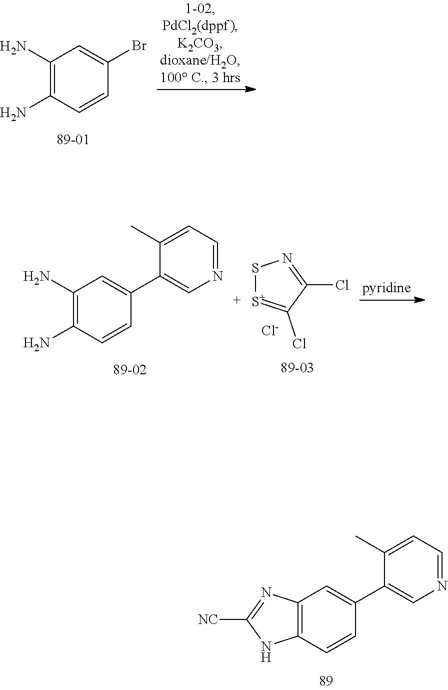 [SMILES: Nc1ccc(Br)cc1N ; Cc1ccncc1-c1ccc(N)c(N)c1 ; Clc1ns[s+]c1Cl ; [Cl-] ; Cc1ccncc1-c1ccc2[nH]c(C#N)nc2c1]

Step 1: The titled compound 89-02 was prepared in a yield of 56.2% according to the procedure outlined for compound 5. Mass (m/z): 200.1 [M+H]⁺.

Step 2: To a solution of 4-(4-methylpyridin-3-yl)benzene-1,2-diamine 89-02 (150 mg, 0.75 mmol) and 4,5-dichloro-1,2,3-dithiazol-1-ium 89-03 (256.9 mg, 0.75 mmol) in pyridine (3 mL) was stirred under nitrogen protection at 25° C. for 12 h. The solvent was removed under vacuum to give a crude product, which was purified by prep-HPLC to give compound 89 (23 mg, yield: 12.9%) as a white solid. Mass (m/z): 235.0 [M+H]⁺. ¹H NMR (400 MHz, DMSO-d₆) δ 14.30 (s, 1H), 8.47-8.43 (m, 2H), 7.83 (d, J=8.5 Hz, 1H), 7.75 (s, 1H), 7.44 (dd, J=8.5, 1.3 Hz, 1H), 7.37 (d, J=5.0 Hz, 1H), 2.28 (s, 3H).

### 6-(4-methoxypyridin-3-yl)benzo[d]oxazol-2(3H)-one (90)

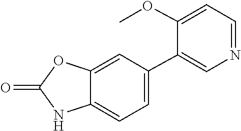 [SMILES: COc1ccncc1-c1ccc2[nH]c(=O)oc2c1]

The titled compound 90 was prepared in a yield of 2.9% according to the procedure outlined for compound 5. Mass (m/z): 243.1 [M+H]⁺. ¹H NMR (400 MHz, DMSO-d₆) δ 11.88 (s, 1H), 8.71 (d, J=30.4 Hz, 2H), 7.67-7.52 (m, 2H), 7.36 (dd, J=8.1, 1.6 Hz, 1H), 7.22 (d, J=8.1 Hz, 1H), 4.05 (s, 3H).

### 7-fluoro-6-(4-methylpyridin-3-yl)benzo[d]oxazol-2(3H)-one (91)

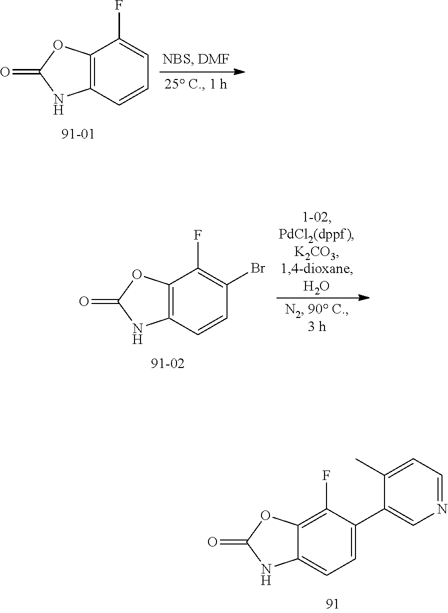 [SMILES: O=c1[nH]c2cccc(F)c2o1 ; O=c1[nH]c2ccc(Br)c(F)c2o1 ; Cc1ccncc1-c1ccc2[nH]c(=O)oc2c1F]

Step 1: To a solution of 7-fluorobenzo[d]oxazol-2(3H)-one 91-01 (306 mg, 2.0 mmol) in DMF (5 mL) was added NBS (356 mg, 2.0 mmol). The mixture was stirred at 25° C. for 1 h. The mixture was diluted with water, extracted with ethyl acetate, washed with brine, dried over Na₂SO₄ and concentrated in vacuo to give crude product. The crude product was purified by silica gel chromatography to give 6-bromo-7-fluorobenzo[d]oxazol-2(3H)-one 91-02 (463 mg, yield: 99%) as a yellow solid. Mass (m/z): 231.9 [M+H]⁺.

Step 2: The titled compound 91 was prepared in a yield of 5.10% according to the procedure outlined for compound 5. Mass (m/z): 245.1 [M+H]⁺. ¹H NMR (400 MHz, DMSO-d₆) δ 12.21 (s, 1H), 8.70-8.55 (m, 2H), 7.71 (d, J=5.5 Hz, 1H), 7.19 (dd, J=8.1, 6.6 Hz, 1H), 7.11 (d, J=8.1 Hz, 1H), 2.31 (s, 3H).

### 5-(4-methylpyridin-3-yl)-1H-indazole 3-carbonitrile (92)

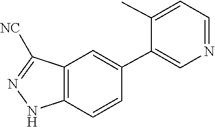 [SMILES: Cc1ccncc1-c1ccc2[nH]nc(C#N)c2c1]

The titled compound 92 was prepared in a yield of 22% according to the procedure outlined for compound 5. Mass (m/z): 235.1 [M+H]⁺. ¹H NMR (400 MHz, DMSO-d₆) δ 14.50 (s, 1H), 8.53-8.35 (m, 2H), 7.92-7.80 (m, 2H), 7.58 (dd, J=8.6, 1.6 Hz, 1H), 7.37 (d, J=5.1 Hz, 1H), 2.28 (s, 3H).

### 5-(4-methylpyridin-3-yl)-3-(trifluoromethyl)-1H-indazole (93)

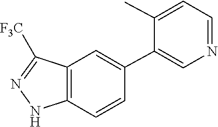 [SMILES: Cc1ccncc1-c1ccc2[nH]nc(C(F)(F)F)c2c1]

The titled compound 93 was prepared in a yield of 26% according to the procedure outlined for compound 5. Mass (m/z): 278.1 [M+H]⁺. ¹H NMR (400 MHz, DMSO-d₆) δ 14.14 (s, 1H), 8.48-8.38 (m, 2H), 7.81 (dd, J=8.7, 0.9 Hz, 11H), 7.77-7.73 (m, 11H), 7.55 (dd, J=8.7, 1.6 Hz, 11H), 7.36 (d, J=5.0 Hz, 1H), 2.26 (s, 3H).

### 6-(4-methylpyridazin-3-yl)benzo[d]oxazol-2(3H)-one (94)

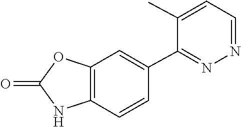 [SMILES: Cc1ccnnc1-c1ccc2[nH]c(=O)oc2c1]

The titled compound 94 was prepared in a yield of 10.7% according to the procedure outlined for compound 5. Mass (m/z): 228.0 [M+H]⁺. ¹H NMR (400 MHz, DMSO-d₆) δ 11.86 (s, 1H), 9.08 (d, J=5.1 Hz, 1H), 7.66 (d, J=5.2 Hz, 1H), 7.59 (d, J=1.5 Hz, 1H), 7.42 (dd, J=8.0, 1.6 Hz, 1H), 7.23 (d, J=8.1 Hz, 1H), 2.36 (s, 3H).

### 6-(5-((3-hydroxyphenyl)amino)pyridin-3-yl)benzo[d]oxazol-2(3H)-one (95)

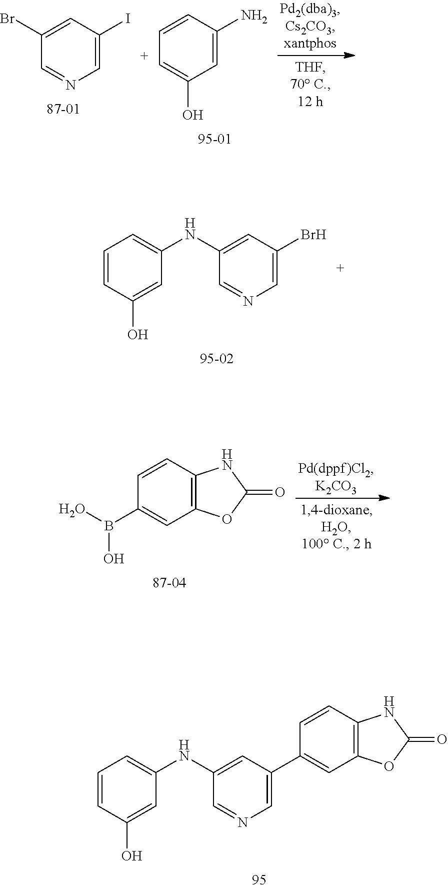 [SMILES: Brc1cncc(I)c1 ; NC1=CC(O)=[C+]C=C1 ; Oc1cccc(Nc2cncc(Br)c2)c1 ; O=c1[nH]c2ccc(B(O)O)cc2o1 ; O=c1[nH]c2ccc(-c3cncc(Nc4cccc(O)c4)c3)cc2o1]

The titled compound 95 was prepared in a yield of 11.6% according to the procedure outlined for compound 87. Mass (m/z): 320.2 [M+H]⁺. ¹H NMR (400 MHz, DMSO-d₆) δ 11.77 (s, 1H), 9.32 (s, 1H), 8.38 (s, 1H), 8.29 (d, J=2.4 Hz, 2H), 7.68-7.58 (m, 2H), 7.44 (dd, J=8.2, 1.7 Hz, 1H), 7.19 (d, J=8.0 Hz, 1H), 7.06 (t, J=7.9 Hz, 1H), 6.63-6.53 (m, 2H), 6.31 (dd, J=8.1, 2.2 Hz, 1H).

### 6-(5-((3-chlorophenyl)amino)pyridin-3-yl)benzo[d]oxazol-2(3H)-one (96)

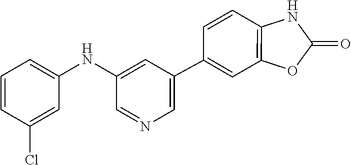 [SMILES: O=c1[nH]c2ccc(-c3cncc(Nc4cccc(Cl)c4)c3)cc2o1]

The titled compound 96 was prepared in a yield of 9.2% according to the procedure outlined for compound 87. Mass (m/z): 338.2 [M+H]⁺. ¹H NMR (400 MHz, DMSO-d₆) δ 8.67 (s, 1H), 8.39 (d, J=2.0 Hz, 1H), 8.34 (d, J=2.5 Hz, 1H), 7.70-7.63 (m, 2H), 7.45 (dd, J=8.1, 1.7 Hz, 1H), 7.29 (t, J=8.0 Hz, 1H), 7.18 (d, J=8.1 Hz, 1H), 7.15-7.07 (m, 2H), 6.91 (dd, J=8.0, 2.0 Hz, 1H).

### 6-(5-((3-methoxyphenyl)amino)pyridin-3-yl)benzo[d]oxazol-2(3H)-one (97)

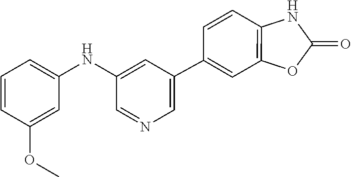 [SMILES: COc1cccc(Nc2cncc(-c3ccc4[nH]c(=O)oc4c3)c2)c1]

The titled compound 97 was prepared in a yield of 11.6% according to the procedure outlined for compound 87. Mass (m/z): 334.1 [M+H]⁺. ¹H NMR (400 MHz, DMSO-d₆) δ 11.81 (s, 1H), 8.48 (s, 1H), 8.37-8.22 (m, 2H), 7.70-7.58 (m, 2H), 7.42 (dd, J=8.1, 1.8 Hz, 1H), 7.19 (t, J=8.3 Hz, 2H), 6.79-6.72 (m, 1H), 6.67 (t, J=2.2 Hz, 1H), 6.49 (dd, J=8.2, 2.4 Hz, 1H), 3.73 (s, 3H).

### 6-(5-(p-tolylamino)pyridin-3-yl)benzo[d]oxazol-2(3H)-one (98)

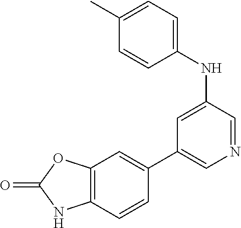 [SMILES: Cc1ccc(Nc2cncc(-c3ccc4[nH]c(=O)oc4c3)c2)cc1]

The titled compound 98 was prepare according to the procedure outlined for compound 87. Mass (m/z): 318.1 [M+H]⁺. ¹H NMR (400 MHz, DMSO-d₆) δ 11.85 (s, 1H), 8.74 (s, 1H), 8.37 (d, J=1.8 Hz, 1H), 8.26 (d, J=2.5 Hz, 1H), 7.79 (s, 1H), 7.71 (d, J=1.7 Hz, 1H), 7.48 (dd, J=8.1, 1.7 Hz, 1H), 7.22 (d, J=8.1 Hz, 1H), 7.19-7.11 (m, 4H), 2.28 (s, 3H).

### 6-(5-((1,2,3,4-tetrahydroquinolin-7-yl)amino)pyridin-3-yl)benzo[d]oxazol-2(3)-one (99)

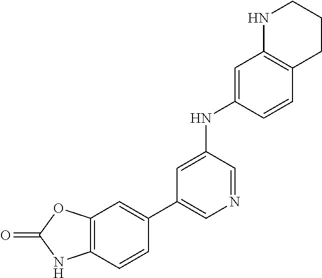 [SMILES: O=c1[nH]c2ccc(-c3cncc(Nc4ccc5c(c4)NCCC5)c3)cc2o1]

The titled compound 99 was prepared in a yield of 58% according to the procedure outlined for compound 87. Mass (m/z): 359.4 [M+H]⁺. ¹H NMR (400 MHz, DMSO-d₆) δ 11.74 (s, 1H), 8.20 (dd, J=13.9, 2.2 Hz, 2H), 8.06 (s, 1H), 7.59 (d, J=1.6 Hz, 1H), 7.50 (t, J=2.3 Hz, 1H), 7.39 (dd, J=8.1, 1.7 Hz, 1H), 7.17 (d, J=8.0 Hz, 1H), 6.73 (d, J=8.0 Hz, 1H), 6.30 (d, J=2.2 Hz, 1H), 6.22 (dd, J=8.0, 2.2 Hz, 1H), 5.68 (d, J=2.5 Hz, 1H), 3.16-3.10 (m, 2H), 2.58 (t, J=6.3 Hz, 2H), 1.82-1.71 (m, 2H).

### 6-(5-((4-fluorophenyl)amino)pyridin-3-yl)benzo[d]oxazol-2(3H)-one (100)

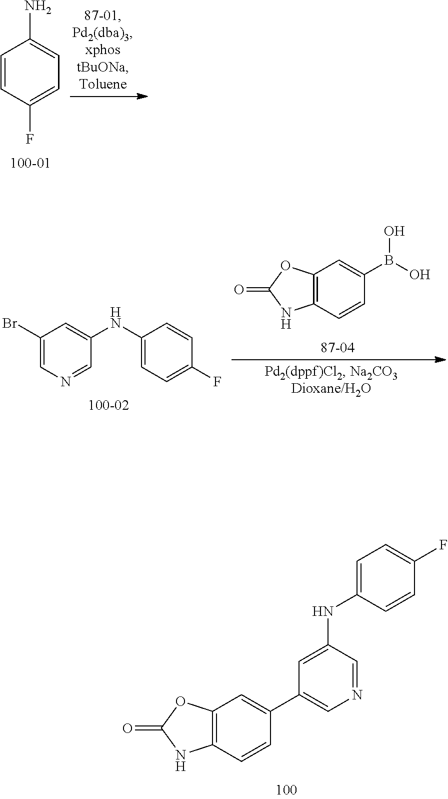 [SMILES: Nc1ccc(F)cc1 ; O=c1[nH]c2ccc(B(O)O)cc2o1 ; Fc1ccc(Nc2cncc(Br)c2)cc1 ; O=c1[nH]c2ccc(-c3cncc(Nc4ccc(F)cc4)c3)cc2o1]

Step 1: A mixture of 4-fluoroaniline 100-01 (111 mg, 1.00 mmol), 3-bromo-5-iodopyridine 87-01 (284 mg, 1.00 mmol), Pd₂(dba)₃ (46 mg, 0.0500 mmol), X-phos (24 mg, 0.05 mmol) and tBuONa (115 mg, 1.20 mmol) in toluene (5 mL) was stirred at 90° C. under nitrogen atmosphere overnight. The reaction mixture was cooled to rt and concentrated to give a crude product which was purified by silica gel chromatography to afford 5-bromo-N-(4-fluorophenyl)pyridin-3-amine 100-02 (70 mg, yield: 25.67%).

Step 2: The titled compound 100 was prepared in a yield of 28.1% according to the procedure outlined for compound 5. Mass (m/z): 322.3 [M+H]⁺. ¹H NMR (400 MHz, DMSO-d₆) δ 11.76 (s, 1H), 8.43 (s, 1H), 8.27 (dd, J=8.8, 2.3 Hz, 2H), 7.63 (d, J=1.7 Hz, 1H), 7.54 (t, J=2.3 Hz, 1H), 7.43 (dd, J=8.1, 1.7 Hz, 1H), 7.24-7.06 (m, 5H).

### 6-(5-((3-fluorophenyl)amino)pyridin-3-yl)benzo[d]oxazol-2(3H)-one (101)

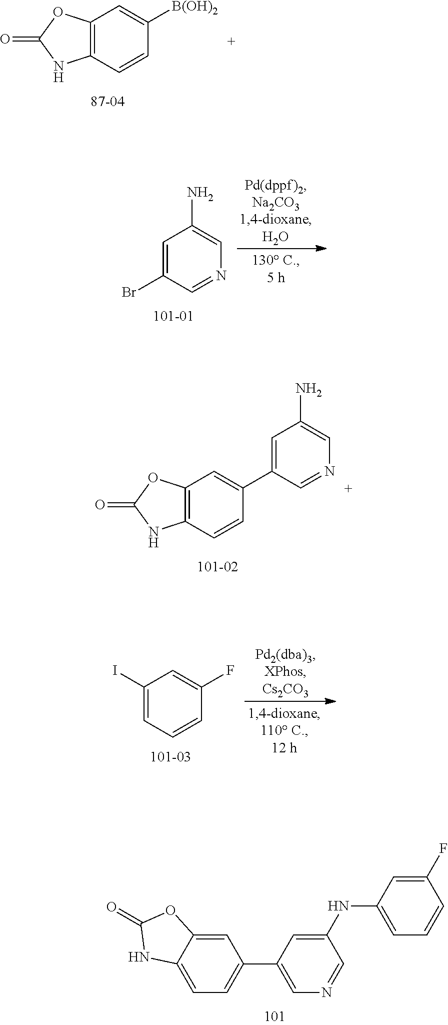 [SMILES: O=c1[nH]c2ccc(B(O)O)cc2o1 ; Nc1cncc(Br)c1 ; Nc1cncc(-c2ccc3[nH]c(=O)oc3c2)c1 ; Fc1cccc(I)c1 ; O=c1[nH]c2ccc(-c3cncc(Nc4cccc(F)c4)c3)cc2o1]

Step 1: The titled compound 101-02 was prepared in a yield of according to the procedure outlined for compound 5. Mass (m/z): 228.1 [M+H]⁺.

Step 2: The titled compound 101 was prepared in a yield of 14.1% according to the procedure outlined for compound 100-02. Mass (m/z): 322.1 [M+H]⁺. ¹H NMR (400 MHz, DMSO-d₆) δ 11.82 (s, 1H), 8.91 (s, 11H), 8.46 (d, J=1.9 Hz, 11H), 8.38 (d, J=2.4 Hz, 11H), 7.85 (t, J=2.2 Hz, 11H), 7.73 (d, J=1.7 Hz, 1H), 7.51 (dd, J=8.1, 1.7 Hz, 1H), 7.32 (dd, J=8.2, 6.9 Hz, 1H), 7.22 (d, J=8.1 Hz, 1H), 7.06-6.94 (m, 2H), 6.74 (d, J=2.4 Hz, 1H).

### 6-(5-((2-fluorophenyl)amino)pyridin-3-yl)benzo[d]oxazol-2(3H)-one (102)

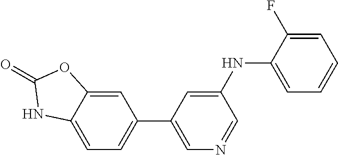 [SMILES: O=c1[nH]c2ccc(-c3cncc(Nc4ccccc4F)c3)cc2o1]

The titled compound 102 was prepared in a yield of 2.6% according to the procedure outlined for compound 100. Mass (m/z): 322.1 [M+H]⁺. ¹H NMR (400 MHz, DMSO-d₆) δ 11.83 (d, J=4.1 Hz, 1H), 8.63 (d, J=20.4 Hz, 1H), 8.42 (s, 1H), 8.27 (d, J=2.4 Hz, 1H), 7.76-7.66 (m, 2H), 7.52-7.39 (m, 2H), 7.31 (ddd, J=11.6, 8.1, 1.7 Hz, 1H), 7.25-7.15 (m, 2H), 7.09 (d, J=6.4 Hz, 1H).

### 6-(5-((3-isopropylphenyl)amino)pyridin-3-yl)benzo[d]oxazol-2(3H)-one (103)

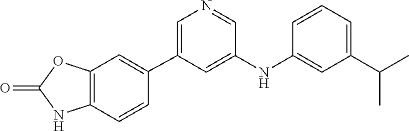 [SMILES: CC(C)c1cccc(Nc2cncc(-c3ccc4[nH]c(=O)oc4c3)c2)c1]

The titled compound 3 was prepared according to the procedure outlined for compound 100. Mass (m/z):346.1 [M+H]⁺. ¹H NMR (400 MHz, DMSO-d₆) δ 11.92 (s, 1H), 9.11 (s, 1H), 8.47 (d, J=1.8 Hz, 1H), 8.32 (d, J=2.5 Hz, 1H), 8.00 (t, J=2.2 Hz, 1H), 7.75 (d, J=1.7 Hz, 1H), 7.52 (dd, J=8.1, 1.8 Hz, 1H), 7.33-7.21 (m, 2H), 7.15-7.07 (m, 2H), 6.95 (d, J=7.6 Hz, 1H), 2.89 (p, J=6.9 Hz, 1H), 1.22 (d, J=6.9 Hz, 6H).

### 6-(5-((4-chlorophenyl)amino)pyridin-3-yl)benzo[d]oxazol-2(3H)-one (104)

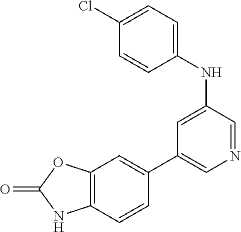 [SMILES: O=c1[nH]c2ccc(-c3cncc(Nc4ccc(Cl)cc4)c3)cc2o1]

The titled compound 104 was prepared according to the procedure outlined for compound 87. Mass (m/z): 338.1 [M+H]⁺. ¹H NMR (400 MHz, DMSO-d₆) δ 11.83 (s, 1H), 8.88 (s, 1H), 8.44 (d, J=1.9 Hz, 1H), 8.33 (d, J=2.5 Hz, 1H), 7.83 (s, 1H), 7.73 (d, J=1.7 Hz, 1H), 7.50 (dd, J=8.1, 1.7 Hz, 1H), 7.40-7.31 (m, 2H), 7.26-7.16 (m, 3H).

### 6-(5-(pyridin-3-ylamino)pyridin-3-yl)benzo[d]oxazol-2(3H)-one (105)

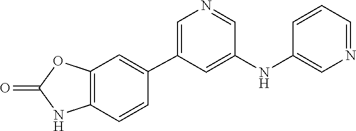 [SMILES: O=c1[nH]c2ccc(-c3cncc(Nc4cccnc4)c3)cc2o1]

The titled compound 105 was prepared according to the procedure outlined for compound 100. Mass (m/z): 305.1 [M+H]⁺. ¹H NMR (400 MHz, DMSO-d₆) δ 11.76 (s, 1H), 8.66 (s, 1H), 8.42 (d, J=2.8 Hz, 1H), 8.37 (d, J=2.0 Hz, 11H), 8.34 (d, J=2.6 Hz, 11H), 8.11 (dd, J=4.7, 1.4 Hz, 1H), 7.68 (d, J=1.7 Hz, 1H), 7.65 (t, J=2.3 Hz, 1H), 7.61 (ddd, J=8.4, 2.9, 1.4 Hz, 1H), 7.47 (dd, J=8.1, 1.7 Hz, 1H), 7.29 (dd, J=8.3, 4.6 Hz, 1H), 7.19 (d, J=8.1 Hz, 1H).

### tert-butyl 7-((5-(2-oxo-2,3-dihydrobenzo[d]oxazol-6-yl)pyridin-3-yl) amino)-3,4-dihydroquinoline-1(2H)-carboxylate (106)

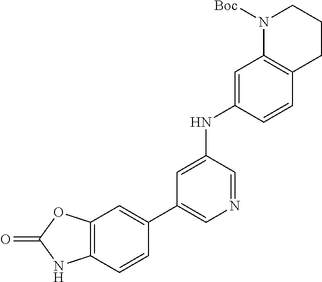 [SMILES: CC(C)(C)OC(=O)N1CCCc2ccc(Nc3cncc(-c4ccc5[nH]c(=O)oc5c4)c3)cc21]

The titled compound 106 was prepared in a yield of 29% according to the procedure outlined for compound 100. Mass (m/z): 459.4 [M+H]⁺. ¹H NMR (400 MHz, DMSO-d₆) δ 11.72 (s, 1H), 8.40 (s, 1H), 8.25 (dd, J=8.6, 2.2 Hz, 2H), 7.77-7.67 (m, 2H), 7.61 (d, J=2.3 Hz, 1H), 7.54 (dd, J=8.1, 1.7 Hz, 1H), 7.14 (d, J=8.1 Hz, 1H), 7.02 (d, J=8.2 Hz, 1H), 6.75 (dd, J=8.1, 2.3 Hz, 1H), 3.68-3.59 (m, 2H), 2.67 (t, J=6.5 Hz, 2H), 1.88-1.76 (m, 2H), 1.47 (s, 9H).

### 3-((5-(2-oxo-2,3-dihydrobenzo[d]oxazol-6-yl)pyridin-3-yl)amino)benzonitrile (107)

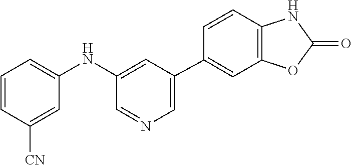 [SMILES: N#Cc1cccc(Nc2cncc(-c3ccc4[nH]c(=O)oc4c3)c2)c1]

The titled compound 107 was prepared in a yield of 11.6% according to the procedure outlined for compound 87. Mass (m/z): 329.1 [M+H]⁺. ¹H NMR (400 MHz, DMSO-d₆) δ 11.83 (s, 1H), 9.00 (s, 1H), 8.49 (d, J=1.9 Hz, 1H), 8.41 (d, J=2.5 Hz, 1H), 7.86 (t, J=2.2 Hz, 1H), 7.74 (d, J=1.6 Hz, 1H), 7.56-7.47 (m, 4H), 7.34 (td, J=4.5, 4.0, 1.5 Hz, 1H), 7.24-7.20 (m, 1H).

### 6-(5-((1-methyl-1H-pyrazol-4-yl)amino)pyridin-3-yl)benzo[d]oxazol-2(3H)-one (108)

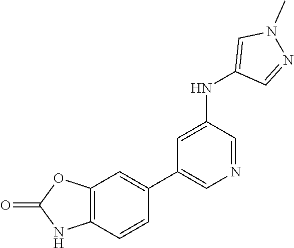 [SMILES: Cn1cc(Nc2cncc(-c3ccc4[nH]c(=O)oc4c3)c2)cn1]

The titled compound 108 was prepared according to the procedure outlined for compound 87. Mass (m/z): 308.1 [M+H]⁺. ¹H NMR (400 MHz, Methanol-d₄) δ 8.22 (s, 1H), 7.95 (s, 1H), 7.80 (t, J=1.8 Hz, 1H), 7.70 (s, 1H), 7.56-7.52 (m, 1H), 7.50-7.41 (m, 2H), 7.16 (d, J=8.1 Hz, 1H), 6.98 (s, 1H), 3.85 (s, 3H).

### 6-(5-(methyl(phenyl)amino)pyridin-3-yl)benzo[d]oxazol-2(3H)-one (109)

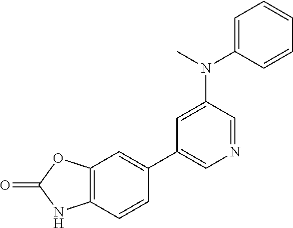 [SMILES: CN(c1ccccc1)c1cncc(-c2ccc3[nH]c(=O)oc3c2)c1]

The titled compound 109 was prepared in a yield of 17% according to the procedure outlined for compound 87. Mass (m/z): 318.3 [M+H]⁺. ¹H NMR (400 MHz, Methanol-d₄) δ 8.32 (d, J=1.7 Hz, 1H), 7.98 (d, J=2.7 Hz, 11H), 7.84 (dd, J=2.7, 1.7 Hz, 11H), 7.60 (d, J=1.8 Hz, 1H), 7.58-7.52 (m, 2H), 7.49 (dd, J=8.1, 1.8 Hz, 1H), 7.42-7.34 (m, 3H), 7.23 (d, J=8.1 Hz, 1H), 3.49 (s, 3H).

### 6-(5-(methylamino)pyridin-3-yl)benzo[d]oxazol-2(3H)-one (110)

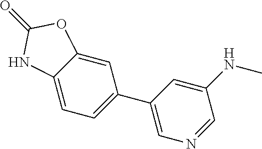 [SMILES: CNc1cncc(-c2ccc3[nH]c(=O)oc3c2)c1]

The titled compound 110 was prepare according to the procedure outlined for compound 87. Mass (m/z): 242.3 [M+H]⁺. ¹H NMR (400 MHz, DMSO-d₆) δ 11.90 (s, 1H), 8.29 (s, 1H), 7.98 (d, J=2.2 Hz, 1H), 7.79 (s, 1H), 7.69-7.47 (m, 2H), 7.24 (d, J=8.1 Hz, 1H), 6.86 (s, 1H), 2.85 (s, 3H)

### 6-(5-(pyridin-4-ylamino)pyridin-3-yl)benzo[d]oxazol-2(3H)-one (111)

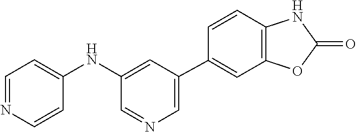 [SMILES: O=c1[nH]c2ccc(-c3cncc(Nc4ccncc4)c3)cc2o1]

The titled compound 111 was prepared in a yield of 12.10% according to the procedure outlined for compound 87. Mass (m/z): 305.2 [M+H]⁺. ¹H NMR (400 MHz, DMSO-d₆) δ 11.85 (s, 1H), 10.63 (s, 1H), 8.84 (d, J=2.1 Hz, 1H), 8.55 (d, J=2.3 Hz, 1H), 8.34 (d, J=7.1 Hz, 2H), 8.09 (t, J=2.3 Hz, 1H), 7.80 (d, J=1.7 Hz, 1H), 7.59 (dd, J=8.1, 1.7 Hz, 1H), 7.27-7.19 (m, 3H).

### 6-(5-(phenylamino)pyridazin-3-yl)benzo[d]oxazol-2(3H)-one (112)

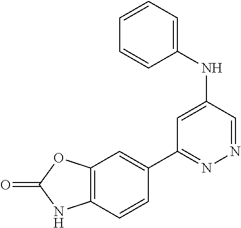 [SMILES: O=c1[nH]c2ccc(-c3cc(Nc4ccccc4)cnn3)cc2o1]

The titled compound 112 was prepared according to the procedure outlined for compound 100. Mass (m/z):305.1 [M+H]⁺. ¹H NMR (400 MHz, DMSO-d₆) δ 9.22 (s, 1H), 8.79 (d, J=2.6 Hz, 1H), 8.36 (s, 1H), 7.88 (s, 1H), 7.76 (d, J=8.2 Hz, 1H), 7.45-7.38 (m, 3H), 7.35-7.29 (m, 2H), 7.18-7.09 (m, 2H).

### 6-(5-(pyridin-2-ylamino)pyridin-3-yl)benzo[d]oxazol-2(3H)-one (113)

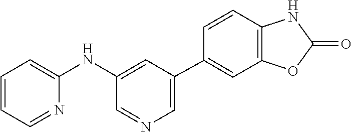 [SMILES: O=c1[nH]c2ccc(-c3cncc(Nc4ccccn4)c3)cc2o1]

The titled compound 113 was prepared according to the procedure outlined for compound 87. Mass (m/z): 305.1 [M+H]⁺. ¹H NMR (400 MHz, DMSO-d₆) δ 11.89 (s, 1H), 9.80 (s, 1H), 9.11 (s, 1H), 8.57 (d, J=2.5 Hz, 2H), 8.27 (dd, J=5.1, 1.9 Hz, 1H), 7.77-7.67 (m, 2H), 7.53 (dd, J=8.1, 1.8 Hz, 1H), 7.27 (d, J=8.1 Hz, 1H), 7.01-6.89 (m, 2H).

### 6-(5-(pyrimidin-5-ylamino)pyridin-3-yl)benzo[d]oxazol-2(3H)-one (114)

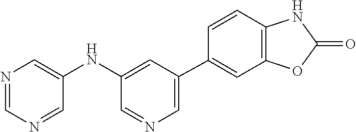 [SMILES: O=c1[nH]c2ccc(-c3cncc(Nc4cncnc4)c3)cc2o1]

The titled compound 114 was prepared in a yield of 16.2% according to the procedure outlined for compound 87. Mass (m/z): 306.1 [M+H]⁺. ¹H NMR (400 MHz, DMSO-d₆) δ 11.86 (s, 1H), 9.08 (d, J=5.5 Hz, 1H), 8.80 (s, 1H), 8.74 (s, 2H), 8.53 (s, 1H), 8.44 (s, 1H), 7.96 (d, J=3.0 Hz, 1H), 7.78 (d, J=1.7 Hz, 1H), 7.55 (dd, J=8.1, 1.8 Hz, 1H), 7.23 (d, J=8.1 Hz, 1H).

### 6-(5-(thiazol-2-ylamino)pyridin-3-yl)benzo[d]oxazol-2(3H)-one (115)

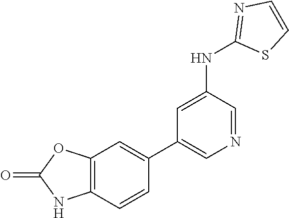 [SMILES: O=c1[nH]c2ccc(-c3cncc(Nc4nccs4)c3)cc2o1]

The titled compound 115 was prepared according to the procedure outlined for compound 87. Mass (m/z): 311.1 [M+H]⁺. ¹H NMR (400 MHz, DMSO-d₆) δ 11.79 (s, 1H), 10.53 (s, 1H), 8.69 (d, J=2.4 Hz, 1H), 8.47 (t, J=2.3 Hz, 1H), 8.44-8.42 (m, 1H), 7.64 (d, J=1.7 Hz, 1H), 7.45 (dd, J=8.1, 1.8 Hz, 1H), 7.33 (d, J=3.7 Hz, 1H), 7.23 (d, J=8.1 Hz, 1H), 7.01 (d, J=3.7 Hz, 1H).

### 6-(5-(pyridin-2-ylamino)pyridin-3-yl)benzo[d]oxazol-2(3H)-one (116)

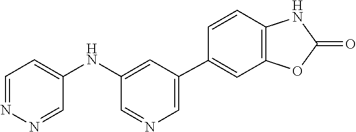 [SMILES: O=c1[nH]c2ccc(-c3cncc(Nc4ccnnc4)c3)cc2o1]

The titled compound 116 was prepared in a yield of 18.1% according to the procedure outlined for compound 87. Mass (m/z): 306.2 [M+H]⁺. ¹H NMR (400 MHz, DMSO-d₆) δ 9.38 (s, 1H), 8.92 (d, J=3.0 Hz, 1H), 8.73 (d, J=6.1 Hz, 1H), 8.61 (d, J=2.0 Hz, 1H), 8.46 (d, J=2.5 Hz, 1H), 7.91 (t, J=2.3 Hz, 1H), 7.76 (d, J=1.7 Hz, 1H), 7.55 (dd, J=8.1, 1.8 Hz, 1H), 7.25 (dd, J=6.1, 3.1 Hz, 11H), 7.21 (d, J=8.1 Hz, 11H).

### 6-(5-((1,3-dimethyl-1H-pyrazol-5-yl)amino)pyridin-3-yl)benzo[d]oxazol-2(3H)-one (117)

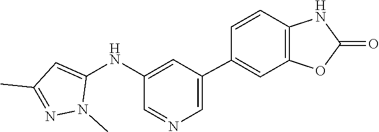 [SMILES: Cc1cc(Nc2cncc(-c3ccc4[nH]c(=O)oc4c3)c2)n(C)n1]

The titled compound 117 was prepared in a yield of 16.2% according to the procedure outlined for compound 87. Mass (m/z): 322.1 [M+H]⁺. ¹H NMR (400 MHz, DMSO-d₆) δ 11.86 (s, 1H), 8.73 (s, 1H), 8.43 (d, J=1.8 Hz, 11H), 8.18 (d, J=2.5 Hz, 11H), 7.71 (d, J=1.7 Hz, 11H), 7.64 (t, J=2.3 Hz, 1H), 7.48 (dd, J=8.1, 1.8 Hz, 1H), 7.23 (d, J=8.1 Hz, 1H), 6.02 (s, 1H), 3.63 (s, 3H), 2.14 (s, 3H).

### 6-(5-(pyridazin-3-ylamino)pyridin-3-yl)benzo[d]oxazol-2(3H)-one (118)

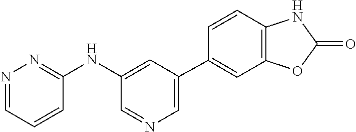 [SMILES: O=c1[nH]c2ccc(-c3cncc(Nc4cccnn4)c3)cc2o1]

The titled compound 118 was prepared in a yield of 38% according to the procedure outlined for compound 87. Mass (m/z): 306.1 [M+H]⁺. ¹H NMR (400 MHz, DMSO-d₆) δ 11.86 (s, 1H), 9.90 (s, 1H), 9.02 (s, 1H), 8.81 (dd, J=4.6, 1.3 Hz, 1H), 8.61 (dd, J=5.2, 3.1 Hz, 2H), 7.72 (d, J=1.7 Hz, 1H), 7.60 (dd, J=9.0, 4.6 Hz, 1H), 7.52 (dd, J=8.1, 1.7 Hz, 1H), 7.33-7.22 (m, 2H).

### 6-(5-phenoxypyridin-3-yl)benzo[d]oxazol-2(3H)-one (119)

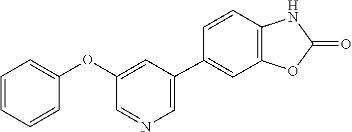 [SMILES: O=c1[nH]c2ccc(-c3cncc(Oc4ccccc4)c3)cc2o1]

The titled compound 119 was prepared in a yield of 10.6% according to the procedure outlined for compound 5. Mass (m/z): 305.2 [M+H]⁺. ¹H NMR (400 MHz, DMSO-d₆) δ 11.81 (s, 1H), 8.72 (d, J=2.0 Hz, 1H), 8.32 (d, J=2.6 Hz, 1H), 7.75 (d, J=2.4 Hz, 2H), 7.52 (dd, J=8.2, 1.8 Hz, 1H), 7.44 (t, J=7.8 Hz, 2H), 7.25-7.17 (m, 2H), 7.13 (d, J=8.0 Hz, 2H).

### 6-(4-methylpyridin-3-yl)quinazoline-2,4(1H,3H)-dione (120)

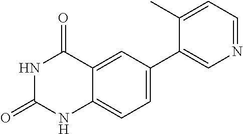 [SMILES: Cc1ccncc1-c1ccc2[nH]c(=O)[nH]c(=O)c2c1]

The titled compound 120 was prepared in a yield of 0.5% according to the procedure outlined for compound 3. Mass (m/z): 254.1 [M+H]⁺. ¹H NMR (400 MHz, DMSO-d₆) δ 8.43 (d, J=5.0 Hz, 1H), 8.38 (s, 1H), 7.83 (d, J=2.1 Hz, 11H), 7.70 (dd, J=8.4, 2.1 Hz, 11H), 7.34 (d, J=5.0 Hz, 1H), 7.27 (d, J=8.4 Hz, 1H), 2.26 (s, 3H)

### Example 2. SAMR1 (50-724) Enzymatic Assay

The enzymatic assay was performed in a 384-well plate using Dulbecco's PBS buffer as the reaction buffer. Purified SARM1 (50-724) with a final concentration of 2 nM was pre-incubated with the test compound at 1% DMSO final assay concentration for 15 min at room temperature. The reaction was initiated by adding the mixture of 200 μM NMN as activator and 100 μM NAD⁺ as substrate. After 1 h of incubation at room temperature, the reaction was terminated with 10 times volume of 70% acetonitrile, then centrifuged at 3800 rpm for 10 min. The samples were analyzed using LC-MS/MS after diluted to the proper concentration by 10 mM ammonium acetate (pH 9.75).

SARM1 inhibitory activity of compounds 1-120 is summarized in Table 2. In Table 2, activity is provided as follows: A=IC50<5 μM; B=5 μM≤IC50<15 μM; C=15 μM≤IC50≤30 μM; D=IC50>30 μM.

TABLE 2

| Cmpd No. | IC50 |
|---|---|
| 1 | B |
| 2 | A |
| 3 | A |
| 4 | A |
| 5 | A |
| 6 | A |
| 7 | A |
| 8 | A |
| 9 | A |
| 10 | C |
| 11 | D |
| 12 | D |
| 13 | A |
| 14 | D |
| 15 | A |
| 16 | A |
| 17 | A |
| 18 | C |
| 19 | B |
| 20 | A |
| 21 | A |
| 22 | A |
| 23 | C |
| 24 | A |
| 25 | A |
| 26 | D |
| 27 | D |
| 28 | A |
| 29 | A |
| 30 | D |
| 31 | A |
| 32 | A |
| 33 | C |
| 34 | D |
| 35 | D |
| 36 | A |
| 37 | B |
| 38 | B |
| 39 | C |
| 40 | A |
| 41 | A |
| 42 | A |
| 43 | B |
| 44 | A |
| 45 | B |
| 46 | D |
| 47 | A |
| 48 | C |
| 49 | D |
| 50 | B |
| 51 | A |
| 52 | B |
| 53 | D |
| 54 | C |
| 55 | C |
| 56 | B |
| 57 | B |
| 58 | B |
| 59 | A |
| 60 | A |
| 61 | B |
| 62 | A |
| 63 | A |
| 64 | A |
| 65 | D |
| 66 | C |
| 67 | C |
| 68 | A |
| 69 | C |
| 70 | C |
| 71 | C |
| 72 | B |
| 73 | A |
| 74 | C |
| 75 | A |
| 76 | A |
| 77 | B |
| 78 | D |
| 79 | A |
| 80 | A |
| 81 | A |
| 82 | C |
| 83 | A |
| 84 | A |
| 85 | B |
| 86 | A |
| 87 | A |
| 88 | A |
| 89 | A |
| 90 | A |
| 91 | C |
| 92 | A |
| 93 | B |
| 94 | A |
| 95 | A |
| 96 | A |
| 97 | A |
| 98 | A |
| 99 | A |
| 100 | A |
| 101 | A |
| 102 | A |
| 103 | A |
| 104 | A |
| 105 | A |
| 106 | A |
| 107 | A |
| 108 | A |
| 109 | A |
| 110 | A |
| 111 | A |
| 112 | A |
| 113 | A |
| 114 | A |
| 115 | A |
| 116 | A |
| 117 | A |
| 118 | A |
| 119 | A |
| 120 | A |

All publications, including but not limited to disclosures and disclosure applications, cited in this specification are herein incorporated by reference as though fully set forth. If certain content of a publication cited herein contradicts or is inconsistent with the present disclosure, the present disclosure controls.

One skilled in the art will readily recognize from the disclosure and claims that various changes, modifications, and variations can be made therein without departing from the spirit and scope of the disclosure as defined in the following claims.

1. A compound of the following structural Formula I: 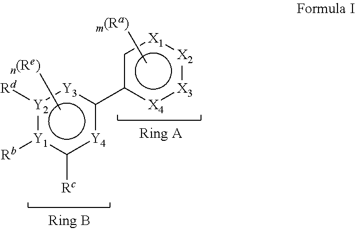
a tautomer thereof, a solvate or stereoisomer of the compound or the tautomer, or a pharmaceutically acceptable salt of the foregoing, wherein:
one or two of X₁, X₂, X₃, and X₄ in Ring A is N, and the rest of X₁, X₂, X₃, and X₄ are C;
Ring B is phenyl or one or two of Y₁, Y₂, Y₃, and Y₄ in Ring B is N, and the rest of Y₁, Y₂, Y₃, and Y₄ are C;
R^{a} is selected from H, —OR^{s}, halogen, —NR^{p}R^{q}, C₃-C₆ cycloalkyl, CN, and C₁-C₆ alkyl wherein the C₃-C₆ cycloalkyl and C₁-C₆ alkyl of R^{a} are optionally substituted with 1 to 3 groups selected from halogen, —OR^{s} and —NR^{p}R^{q},
R^{c} is selected from H, CN, —S(═O)_{w}NR^{p1}R^{q1}, —OR^{s}, halogen, —NR^{p1}R^{q1}, and C₁-C₆ alkyl optionally substituted with 1 to 3 groups selected from halogen, —OR^{s} and —NR^{p1}R^{q1}, provided that R^{c} is not CH₂OH;
R^{b} is absent or selected from H, halogen, —C(═O)(C₁-C₆ alkyl), —NR^{p1}R^{q1}, —OR^{s}, and C₁-C₆ alkyl optionally substituted with 1 to 3 groups selected from halogen, —OR^{s}, and —NR^{p1}R^{q1};
R^{d} is absent or selected from H, CN, —S(═O)_{w}NR^{p1}R^{q1}, —OR^{s}, halogen, —NR^{p1}R^{q1}, C₃-C₆ cycloalkyl and C₁-C₆ alkyl optionally substituted with 1 to 3 groups selected from halogen, —OR^{s} and —NR^{p1}R^{q1}, provided that R^{d} is not CH₂OH;
R^{b} and R^{c} may join together to form an optionally substituted 5- to 7-membered heterocyclic or heteroaromatic ring;
R^{b} and R^{d} may join together to form an optionally substituted 5- to 7-membered heterocyclic or heteroaromatic ring;
R^{e} is selected from H, halogen, —CN, C₁-C₆ alkyl optionally substituted with 1 to 3 groups selected from halogen, —OR^{s} and —NR^{p1}R^{q1};
R^{s}, for each occurrence, is independently selected from H, phenyl, 5-10 membered heteroaryl, C₃-C₆ cycloalkyl and C₁-C₆ alkyl, wherein the phenyl, 5-10 membered heteroaryl, C₃-C₆ cycloalkyl, and C₁-C₆ alkyl of R^{s} are each optionally substituted with 1-3 groups selected from halogen, —OH and —O(C₁-C₃ alkyl);
R^{p} and R^{q}, for each occurrence, are independently selected from hydrogen, phenyl, 9-10 membered aryl, 5-10 membered heteroaryl, C₃-C₆ cycloalkyl, and C₁-C₄ alkyl, wherein the phenyl, 9-10 membered aryl, 5-10 membered heteroaryl, C₃-C₆ cycloalkyl, and C₁-C₄ alkyl of R^{p} and R^{q} are each optionally substituted with 1 to 3 groups selected from —COO(C₁-C₄ alkyl), halogen, —OH, —CN, —COOH, —CONH₂, —CONH(C₁-C₃ alkyl), —NH(C₁-C₃ alkyl), —O(C₁-C₃ alkyl), and C₁-C₄ alkyl;
R^{p1} and R^{q1}, for each occurrence, are independently selected from hydrogen, —CO(C₁-C₆ alkyl), —SO₂CH₃, C₃-C₆ cycloalkyl, and C₁-C₆ alkyl, wherein the C₃-C₆ cycloalkyl and C₁-C₆ alkyl of R^{p1} and R^{q1} are each optionally substituted with 1 to 3 halogens;
m is an integer independently selected from 0, 1, 2, and 3;
n is an integer independently selected from 0, 1, and 2; and
w is an integer selected from 1 and 2.

2. The compound, tautomer, solvate, stereoisomer, or pharmaceutically acceptable salt of claim 1, wherein Ring A is a pyridinyl, pyrimidinyl, or pyridazinyl group.

3. The compound, tautomer, solvate, stereoisomer, or pharmaceutically acceptable salt of claim 1, wherein Ring B is a phenyl or a pyridinyl group, wherein one of Y₁, Y₂, Y₃, and Y₄ is N, and the rest of Y₁, Y₂, Y₃, and Y₄ are C.

4. The compound of claim 1, wherein the compound has a structure selected from the following structural Formulae IIa to IId: 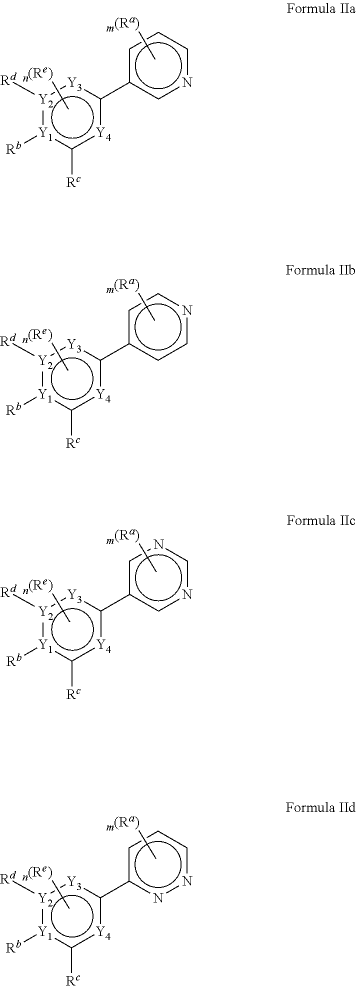
a tautomer thereof, a solvate or stereoisomer of the compound or the tautomer, or a pharmaceutically acceptable salt of the foregoing.

5.-7. (canceled)

8. The compound of claim 1, wherein the compound has a structure selected from the following structural Formulae IIIa-IIIe: 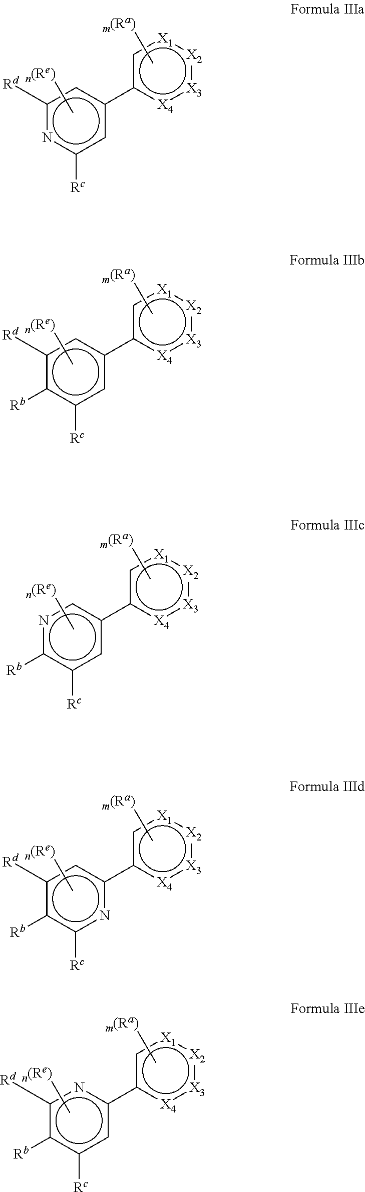
a tautomer thereof, a solvate or stereoisomer of the compound or the tautomer, or a pharmaceutically acceptable salt of the foregoing.

9.-12. (canceled)

13. The compound of claim 1, wherein the compound has the following structural Formula IIIf: 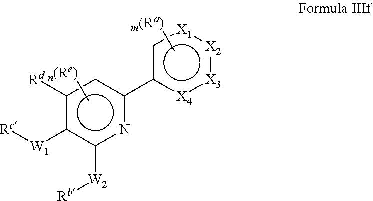
a tautomer thereof, a solvate or stereoisomer of the compound or the tautomer, or a pharmaceutically acceptable salt of the foregoing, wherein W₁ and W₂ are each independently selected from NH and O; and wherein
when W₁ is O, R^{c′} is selected from H, C₃-C₆ cycloalkyl, and C₁-C₆ alkyl, wherein the C₃-C₆ cycloalkyl, and C₁-C₆ alkyl of R^{c′} are each optionally substituted with 1-3 groups selected from halogen, —OH and —O(C₁-C₃alkyl);
when W₁ is NH, R^{c′} is selected from hydrogen, —CO(C₁-C₆ alkyl), —SO₂CH₃, C₃-C₆ cycloalkyl, and C₁-C₆ alkyl, wherein the C₃-C₆ cycloalkyl and C₁-C₆ alkyl of R^{c′} are each optionally substituted with 1 to 3 halogens;
when W₂ is O, R^{b′} is selected from H, C₃-C₆ cycloalkyl and C₁-C₆ alkyl, wherein the C₃-C₆ cycloalkyl and C₁-C₆ alkyl of R^{b′} are each optionally substituted with 1-3 groups selected from halogen, —OH and —O(C₁-C₃alkyl);
when W₂ is NH, R^{b′} is selected from hydrogen, —CO(C₁-C₆ alkyl, —SO₂CH₃, C₃-C₆ cycloalkyl, and C₁-C₆ alkyl, wherein the C₃-C₆ cycloalkyl and C₁-C₆ alkyl of R^{b′} are each optionally substituted with 1 to 3 halogens.

14. The compound of claim 1, wherein the compound has a structure selected from the following structural Formulae IVa-IVd: 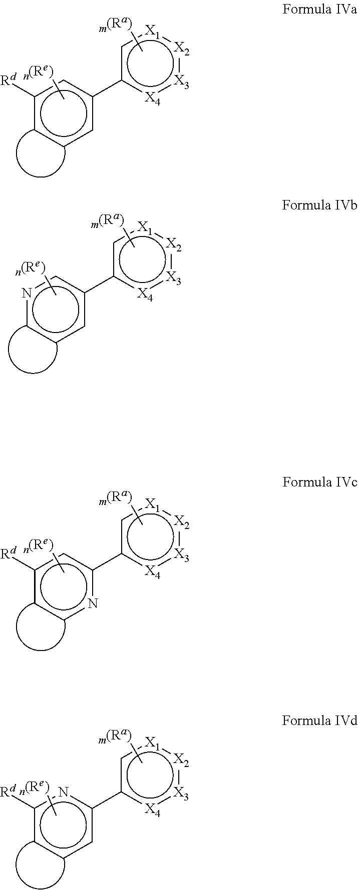
a tautomer thereof, a solvate or stereoisomer of the compound or the tautomer, or a pharmaceutically acceptable salt of the foregoing, wherein R^{b} and R^{c} join together to form an optionally substituted 5- to 7-membered heterocyclic or heteroaromatic ring.

15.-17. (canceled)

18. The compound of claim 1, wherein the compound has one of the following structural Formula Va: 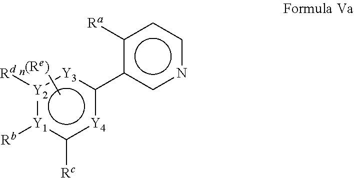
a tautomer thereof, a solvate or stereoisomer of the compound or the tautomer, or a pharmaceutically acceptable salt of the foregoing, wherein R^{a} is C₁-C₃ alkyl, halogen, —O(C₁-C₃)alkyl, —OCFH₂, —OCF₂H, —OCF₃.

19. The compound of claim 1, wherein the compound has one of the following structural Formula VIa: 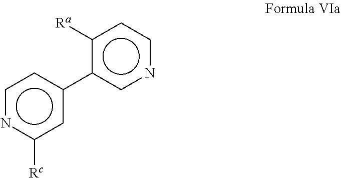
a tautomer thereof, a solvate or stereoisomer of the compound or the tautomer, or a pharmaceutically acceptable salt of the foregoing, wherein R^{c} is —OR^{s}.

20. The compound of claim 1, wherein the compound has one of the following structural Formula VIb: 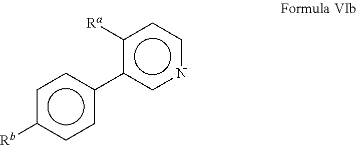
a tautomer thereof, a solvate or stereoisomer of the compound or the tautomer, or a pharmaceutically acceptable salt of the foregoing, wherein R^{b} is —OR^{s}.

21. The compound of claim 1, wherein the compound has one of the following structural Formula VIc: 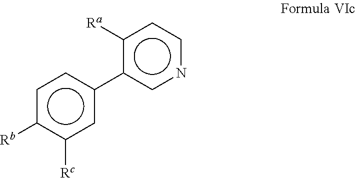
a tautomer thereof, a solvate or stereoisomer of the compound or the tautomer, or a pharmaceutically acceptable salt of the foregoing, wherein R^{b} is —OR^{s}; and R^{c} is selected from halogen, —OR^{s}, and C₁-C₃ alkyl.

22. The compound of claim 1, wherein the compound has one of the following structural Formula VId: 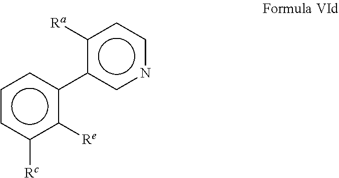
a tautomer thereof, a solvate or stereoisomer of the compound or the tautomer, or a pharmaceutically acceptable salt of the foregoing, wherein R^{c} is —OR^{s} and R^{e} is selected from halogen.

23. The compound of claim 1, wherein the compound has one of the following structural Formula VIe: 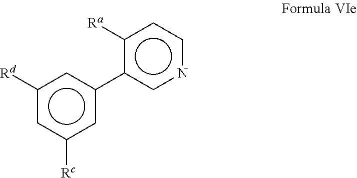
a tautomer thereof, a solvate or stereoisomer of the compound or the tautomer, or a pharmaceutically acceptable salt of the foregoing, wherein R^{c} and R^{d} are each independently selected from —OR^{s} and halogen.

24. The compound of claim 1, wherein the compound has one of the following structural Formula VIf: 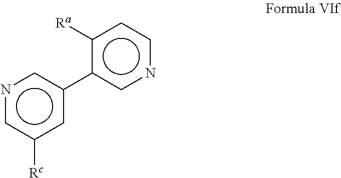
a tautomer thereof, a solvate or stereoisomer of the compound or the tautomer, or a pharmaceutically acceptable salt of the foregoing, wherein R^{c} is selected from —OR^{s} and halogen.

25. The compound of claim 1, wherein the compound has one of the following structural Formula VIIa: 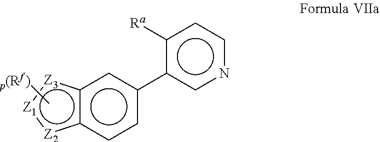
a tautomer thereof, a solvate or stereoisomer of the compound or the tautomer, or a pharmaceutically acceptable salt of the foregoing, wherein R^{a} is selected from H, —OR^{s}, halogen, —NR^{p}R^{q}, C₃-C₆ cycloalkyl, CN, and C₁-C₆ alkyl optionally substituted with 1 to 3 groups selected from halogen, —OR^{s} and —NR^{p}R^{q}, wherein R^{p} and R^{q}, for each occurrence, are independently selected from hydrogen, C₁-C₃ alkyl, phenyl, 9-10 membered aryl, 5-10 membered heteroaryl, wherein the phenyl, 9-10 membered aryl, and 5-10 membered heteroaryl of R^{p} and R^{q} are optionally substituted with 1 to 3 groups selected from —COO(C₁-C₄ alkyl), halogen, OH, —CN, —COOH, —CONH₂, —CONH(C₁-C₃ alkyl), —NH(C₁-C₃ alkyl), —O(C₁-C₃ alkyl), and C₁-C₄ alkyl; R^{f} is selected from halogen, C₁-C₄ alkyl, C₂-C₄ alkynyl, and CN, p is an integer selected from 0, 1, and 2; one, two, or three of Z₁, Z₂, and Z₃ are N, and when one or two of Z₁, Z₂, and Z₃ are N, the rest of Z₁, Z₂, and Z₃ is C.

26. The compound of claim 1, wherein the compound has one of the following structural Formula VIIb: 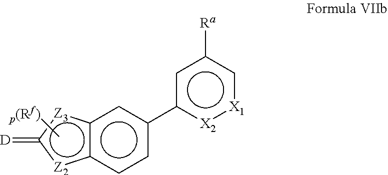
a tautomer thereof, a solvate or stereoisomer of the compound or the tautomer, or a pharmaceutically acceptable salt of the foregoing, wherein R^{a} is selected from H, —OR^{s}, halogen, —NR^{p}R^{q}, C₃-C₆ cycloalkyl, CN, and C₁-C₆ alkyl optionally substituted with 1 to 3 groups selected from halogen, —OR^{s} and —NR^{p}R^{q}, wherein R^{p} and R^{q}, for each occurrence, are independently selected from hydrogen, C₁-C₃ alkyl, phenyl, 9-10 membered aryl, 5-10 membered heteroaryl, wherein the phenyl, 9-10 membered aryl, and 5-10 membered heteroaryl of R^{p} and R^{q} are optionally substituted with 1 to 3 groups selected from —COO(C₁-C₄ alkyl), halogen, OH, —CN, —COOH, —CONH₂, —CONH(C₁-C₃ alkyl), —NH(C₁-C₃ alkyl), —O(C₁-C₃ alkyl), and C₁-C₄ alkyl; R^{f} is selected from halogen, C₁-C₄ alkyl, C₂-C₄ alkynyl, and ═O, p is an integer selected from 0, 1, and 2; Z₂ and Z₃ are each independently selected from O, N, S, and C wherein at least one of Z₂ and Z₃ is a heteroatom; D is selected from 0, S, and NH; one or two of X₁ and X₂ are N.

27. The compound of claim 1, wherein the compound has one of the following structural Formula VIIc: 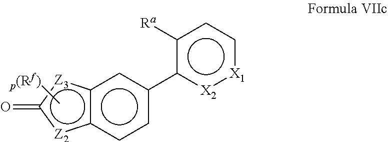
a tautomer thereof, a solvate or stereoisomer of the compound or the tautomer, or a pharmaceutically acceptable salt of the foregoing, wherein R^{a} is selected from H, —OR^{s}, halogen, —NR^{p}R^{q}, C₃-C₆ cycloalkyl, CN, and C₁-C₆ alkyl optionally substituted with 1 to 3 groups selected from —COO(C₁-C₄ alkyl), halogen, —OR^{s} and —NR^{p}R^{q}, wherein R^{p} and R^{q}, for each occurrence, are independently selected from hydrogen, C₁-C₃ alkyl, phenyl, 9-10 membered aryl, 5-10 membered heteroaryl, wherein the phenyl, 9-10 membered aryl, and 5-10 membered heteroaryl of R^{p} and R^{q} are optionally substituted with 1 to 3 groups selected from halogen, OH, —CN, —COOH, —CONH₂, —CONH(C₁-C₃ alkyl), —NH(C₁-C₃ alkyl), —O(C₁-C₃ alkyl), and C₁-C₄ alkyl; R^{f} is selected from halogen C₁-C₄ alkyl, C₂-C₄ alkynyl, and ═O, p is an integer selected from 0, 1, and 2; Z₂ and Z₃ are each independently selected from O, N, S, and C wherein at least one of Z₂ and Z₃ is a heteroatom; D is selected from 0, S, and NH; one or two of X₁ and X₂ are N.

28. The compound of claim 1, wherein the compound has one of the following structural Formula VIId: 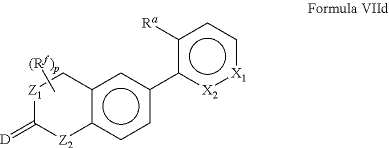
a tautomer thereof, a solvate or stereoisomer of the compound or the tautomer, or a pharmaceutically acceptable salt of the foregoing, wherein R^{a} is selected from H, —OR^{s}, halogen, —NR^{p}R^{q}, C₃-C₆ cycloalkyl, CN, and C₁-C₆ alkyl optionally substituted with 1 to 3 groups selected from halogen, —OR^{s} and —NR^{p}R^{q}, wherein R^{p} and R^{q}, for each occurrence, are independently selected from —COO(C₁-C₄ alkyl), hydrogen, C₁-C₃ alkyl, phenyl, 9-10 membered aryl, 5-10 membered heteroaryl, wherein the phenyl, 9-10 membered aryl, and 5-10 membered heteroaryl of R^{p} and R^{q} are optionally substituted with 1 to 3 groups selected from halogen, OH, —CN, —COOH, —CONH₂, —CONH(C₁-C₃ alkyl), —NH(C₁-C₃alkyl), —O(C₁-C₃ alkyl), and C₁-C₄ alkyl; R^{f} is selected from halogen, C₁-C₄ alkyl, C₂-C₄ alkynyl, and ═O, p is an integer selected from 0, 1, and 2; Z₁ and Z₂ are each independently selected from O, N, S, and C wherein at least one of Z₁ and Z₂ is a heteroatom; D is selected from 0, NH, and S; one or two of X₁ and X₂ are N.

29. The compound of claim 1, wherein the compound has one of the following structural Formula VIIe: 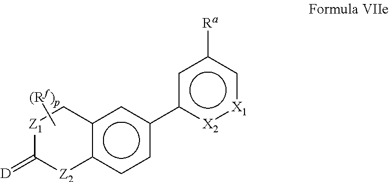
a tautomer thereof, a solvate or stereoisomer of the compound or the tautomer, or a pharmaceutically acceptable salt of the foregoing, wherein R^{a} is selected from H, —OR^{s}, halogen, —NR^{p}R^{q}, C₃-C₆ cycloalkyl, CN, and C₁-C₆ alkyl optionally substituted with 1 to 3 groups selected from halogen, —OR^{s} and —NR^{p}R^{q}, wherein R^{p} and R^{q}, for each occurrence, are independently selected from hydrogen, C₁-C₃ alkyl, phenyl, 9-10 membered aryl, 5-10 membered heteroaryl, wherein the phenyl, 9-10 membered aryl, and 5-10 membered heteroaryl of R^{p} and R^{q} are optionally substituted with 1 to 3 groups selected from —COO(C₁-C₄ alkyl), halogen, OH, —CN, —COOH, —CONH₂, —CONH(C₁-C₃ alkyl), —NH(C₁-C₃ alkyl), —O(C₁-C₃ alkyl), and C₁-C₄ alkyl; R^{f} is selected from halogen, C₁-C₄ alkyl, C₂-C₄ alkynyl, and ═O, p is an integer selected from 0, 1, and 2; Z₁ and Z₂ are each independently selected from O, N, S, and C wherein at least one of Z₁ and Z₂ is a heteroatom; D is selected from 0, NH, and S; one or two of X₁ and X₂ are N.

30. The compound, tautomer, solvate, stereoisomer, or pharmaceutically acceptable salt of claim 1, wherein R^{a} is selected from H, —OR^{s}, halogen, —NR^{p}R^{q}, C₃-C₆ cycloalkyl, CN, and C₁-C₆ alkyl optionally substituted with 1 to 3 groups selected from halogen, —OR^{s}, and —NR^{p}R^{q}, wherein R^{s}, for each occurrence, is independently selected from H, phenyl, —CFH₂, —CF₂H, —CF₃ and C₁-C₃ alkyl, and R^{p} and R^{q}, for each occurrence, are independently selected from hydrogen, C₁-C₃ alkyl, phenyl, 9-10 membered aryl, 5-10 membered heteroaryl, wherein the phenyl, 9-10 membered aryl, and 5-10 membered heteroaryl of R^{p} and R^{q} are optionally substituted with 1 to 3 groups selected from —COO(C₁-C₄ alkyl), halogen, OH, —CN, —COOH, —CONH₂, —CONH(C₁-C₃ alkyl), —NH(C₁-C₃ alkyl), —O(C₁-C₃ alkyl), and C₁-C₄ alkyl.

31. The compound, tautomer, solvate, stereoisomer, or pharmaceutically acceptable salt of claim 1, wherein R^{a} is selected from H, —CH₃, —CH₂CH₃, —OCFH₂, —OCF₂H, —OCF₃, —OCH₃, CN, C₁, OH, NH₂, —NHCH₃, and

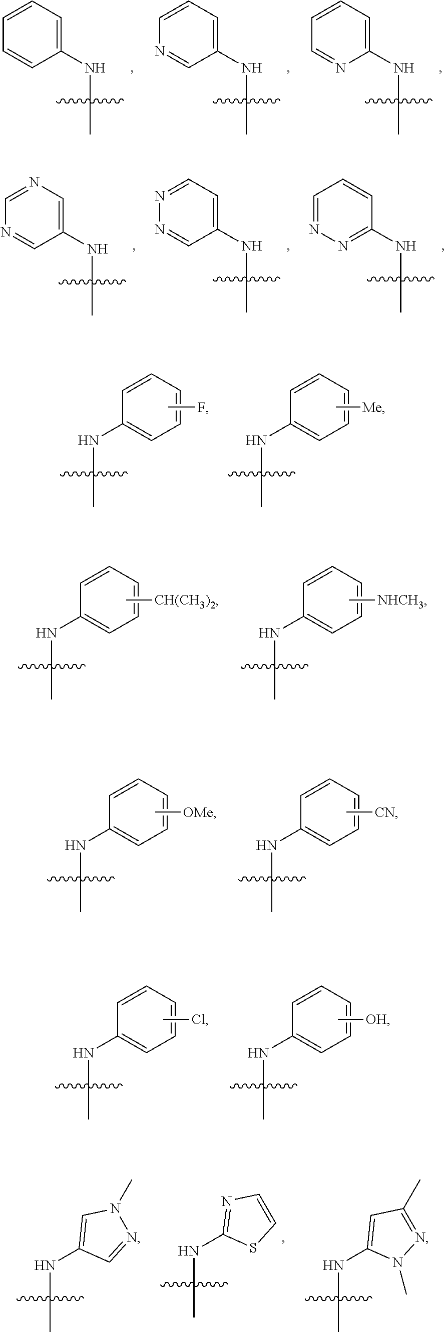

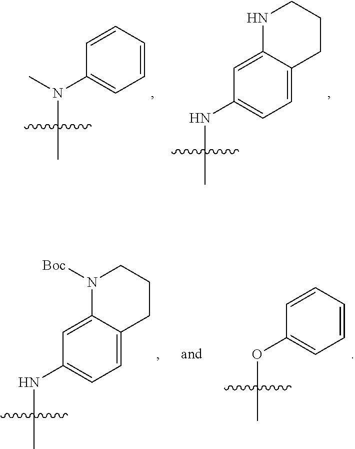

32. The compound, tautomer, solvate, stereoisomer, or pharmaceutically acceptable salt of claim 1, wherein R^{b} is absent or selected from H, halogen, —C(═O)(C₁-C₃ alkyl), —NR^{p1}R^{q1}, —OR^{s}, and C₁-C₃ alkyl optionally substituted with 1 to 2 groups selected from halogen and —NR^{p1}R^{q1}, wherein R^{s}, for each occurrence, is independently selected from H, —CF₃, —CF₂H, and C₁-C₃ alkyl, and R^{p1} and R^{q1}, for each occurrence, are independently selected from hydrogen and optionally substituted C₁-C₃ alkyl.

33. The compound, tautomer, solvate, stereoisomer, or pharmaceutically acceptable salt of claim 1, wherein R^{b} is selected from H, methyl, CH₂NH₂, —C(═O)CH₃, NH₂, F, Br, OH, and

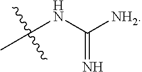 [SMILES: *NC(=N)N]

34. The compound, tautomer, solvate, stereoisomer, or pharmaceutically acceptable salt of claim 1, wherein R^{c} is selected from H, CN, —S(═O)_{w}NR^{p}R^{q}, —OR^{s}, halogen, —NR^{p1}R^{q1}, and C₁-C₃ alkyl optionally substituted with 1 to 3 groups selected from halogen and —NR^{p1}R^{q1}, wherein R^{s}, for each occurrence, is independently selected from H and C₁-C₃ alkyl, and R^{p1} and R^{q1}, for each occurrence, are independently selected from hydrogen and C₁-C₃ alkyl.

35. The compound, tautomer, solvate, stereoisomer, or pharmaceutically acceptable salt of claim 1, wherein R^{c} is selected from H, methyl, ethyl, CHF₂, CF₃, F, Cl, Br, NH₂, OH, OCH₃, CN, and —S(═O)₂NH₂.

36. The compound, tautomer, solvate, stereoisomer, or pharmaceutically acceptable salt of claim 1, wherein R^{d} is selected from H, CN, —S(═O)_{w}NR^{p}R^{q}, —OR^{s}, halogen, —NR^{p1}R^{q1}, and C₁-C₃ alkyl optionally substituted with 1 to 3 groups selected from halogen and —NR^{p1}R^{q1}, wherein R^{s}, for each occurrence, is independently selected from H and C₁-C₃ alkyl, and R^{p1} and R^{q1}, for each occurrence, are independently selected from hydrogen and C₁-C₃ alkyl.

37. The compound, tautomer, solvate, stereoisomer, or pharmaceutically acceptable salt of claim 1, wherein R^{d} is selected from H, methyl, ethyl, CHF₂, CF₃, F, Cl, Br, NH₂, OH, OCH₃, CN, and —S(═O)₂NH₂.

38. The compound, tautomer, solvate, stereoisomer, or pharmaceutically acceptable salt of claim 1, wherein R^{e} is selected from H, halogen, and C₁-C₃ alkyl optionally substituted with 1 to 3 groups selected from halogen and —NH₂.

39. The compound, tautomer, solvate, stereoisomer, or pharmaceutically acceptable salt of claim 1, wherein R^{e} is selected from H, methyl, F, and Cl.

40. The compound, tautomer, solvate, stereoisomer, or pharmaceutically acceptable salt of claim 1, wherein R^{b} and R^{c}, or R^{b} and R^{d}, join to form a 5- to 7-membered heterocyclic or heteroaromatic ring optionally substituted with 1 to 2 groups selected from CN, halogen, ═O, ═S, ═NH, C₁-C₃ alkyl optionally substituted with 1 to 3 groups selected from halogen, and —NR^{p}R^{q}, wherein R^{p} and R^{q}, for each occurrence, are independently selected from hydrogen, C₁-C₃ alkyl, and —C(═O)C₁-C₃ alkyl.

41. The compound, tautomer, solvate, stereoisomer, or pharmaceutically acceptable salt of claim 1, wherein R^{b} and R^{c}, or R^{b} and R^{d}, join to form a structure selected from:

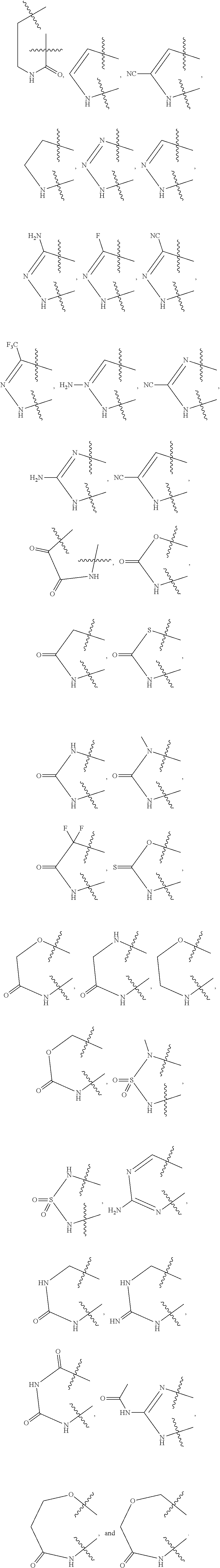

42. The compound of claim 1, wherein the compound has one of the following structural Formula VIIIa: 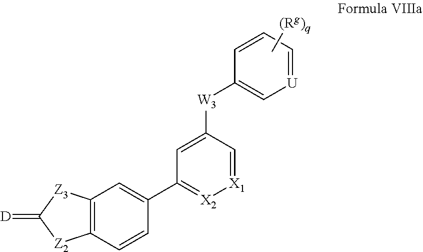
a tautomer thereof, a solvate or stereoisomer of the compound or the tautomer, or a pharmaceutically acceptable salt of the foregoing, wherein R^{g}, for each occurrence, is selected from C₁-C₃ alkyl, ON, OH, —O(C₁-C₃ alkyl), —NH(C₁-C₃ alkyl), halogen, q is an integer selected from 0, 1, and 2; Z₁ and Z₂ are each independently selected from 0, NH, S, and CH₂ wherein at least one of Z₂ and Z₃ is a heteroatom; D is selected from 0 and S; one or two of X, and X₂ are N; U is C or N; W₃ is O or NH.

43. The compound according to claim 1, wherein the compound is selected from: 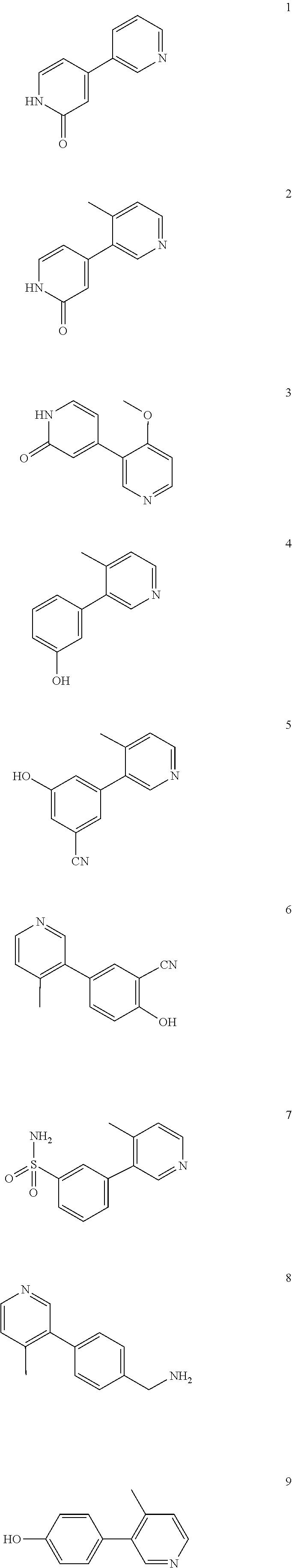 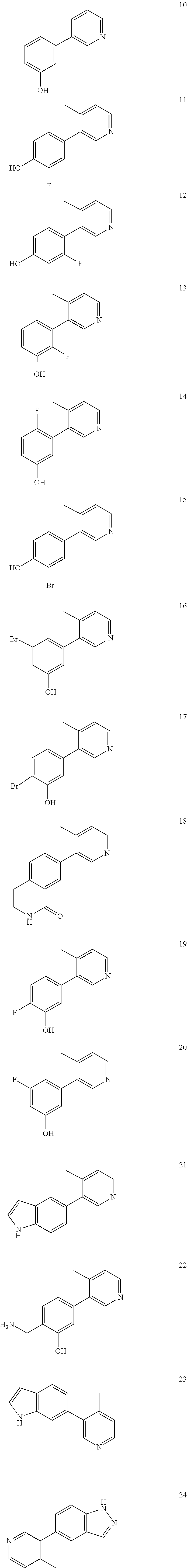 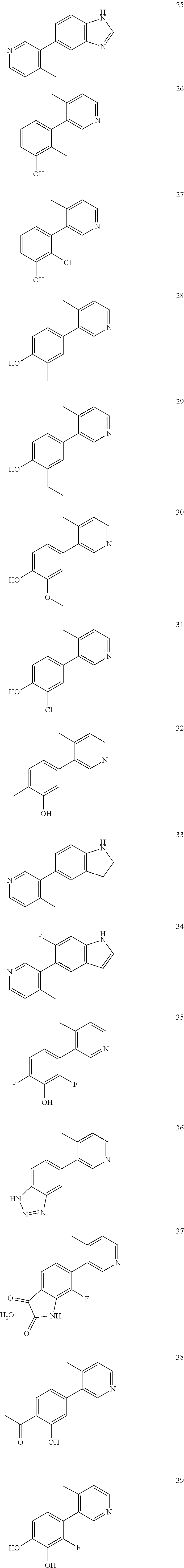 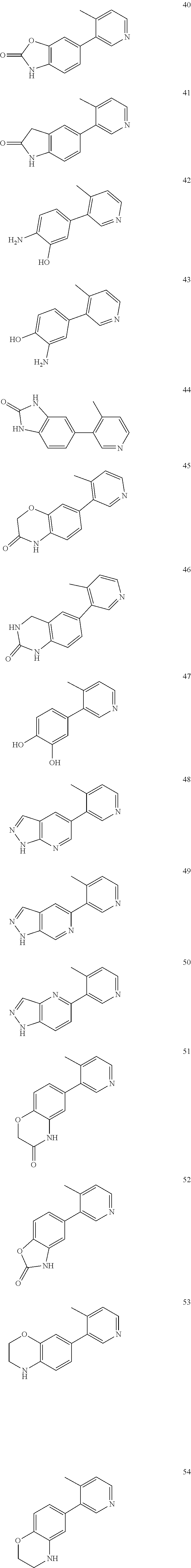 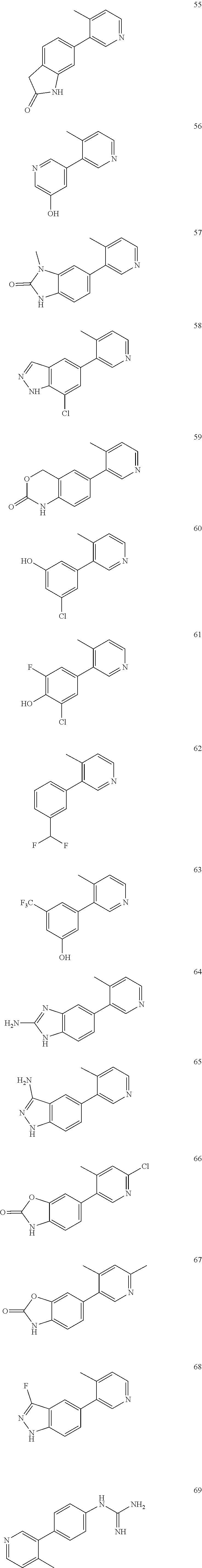 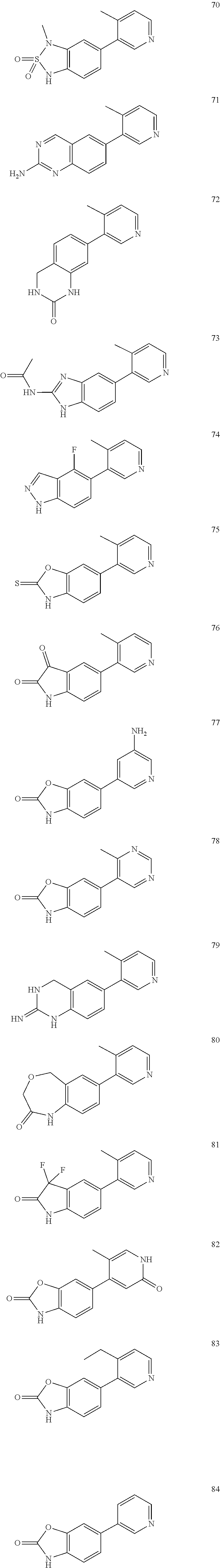 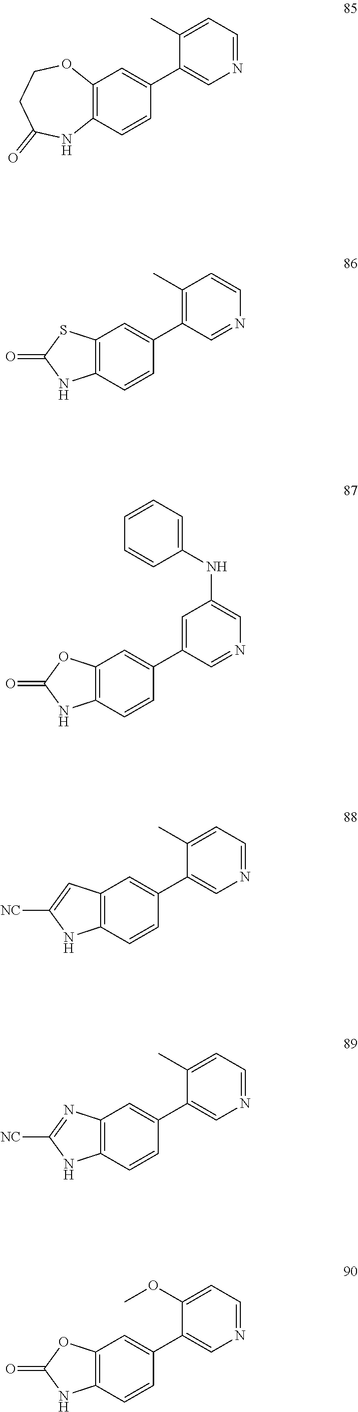 [SMILES: Cc1ccncc1-c1ccc2c(c1)OCCC(=O)N2 ; Cc1ccncc1-c1ccc2[nH]c(=O)sc2c1 ; O=c1[nH]c2ccc(-c3cncc(Nc4ccccc4)c3)cc2o1 ; Cc1ccncc1-c1ccc2[nH]c(C#N)cc2c1 ; Cc1ccncc1-c1ccc2[nH]c(C#N)nc2c1 ; COc1ccncc1-c1ccc2[nH]c(=O)oc2c1] 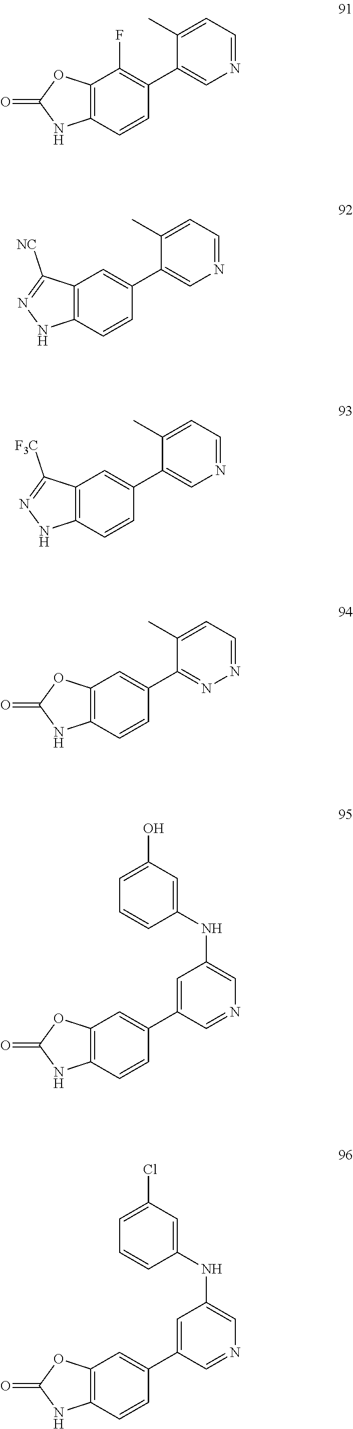 [SMILES: Cc1ccncc1-c1ccc2[nH]c(=O)oc2c1F ; Cc1ccncc1-c1ccc2[nH]nc(C#N)c2c1 ; Cc1ccncc1-c1ccc2[nH]nc(C(F)(F)F)c2c1 ; Cc1ccnnc1-c1ccc2[nH]c(=O)oc2c1 ; O=c1[nH]c2ccc(-c3cncc(Nc4cccc(O)c4)c3)cc2o1 ; O=c1[nH]c2ccc(-c3cncc(Nc4cccc(Cl)c4)c3)cc2o1] 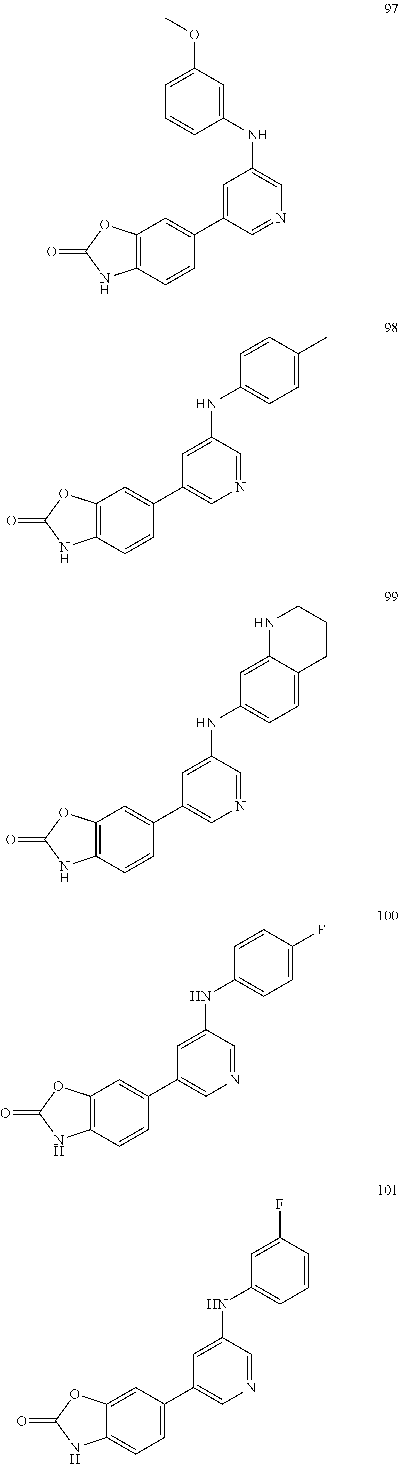 [SMILES: COc1cccc(Nc2cncc(-c3ccc4[nH]c(=O)oc4c3)c2)c1 ; Cc1ccc(Nc2cncc(-c3ccc4[nH]c(=O)oc4c3)c2)cc1 ; O=c1[nH]c2ccc(-c3cncc(Nc4ccc5c(c4)NCCC5)c3)cc2o1 ; O=c1[nH]c2ccc(-c3cncc(Nc4ccc(F)cc4)c3)cc2o1 ; O=c1[nH]c2ccc(-c3cncc(Nc4cccc(F)c4)c3)cc2o1] 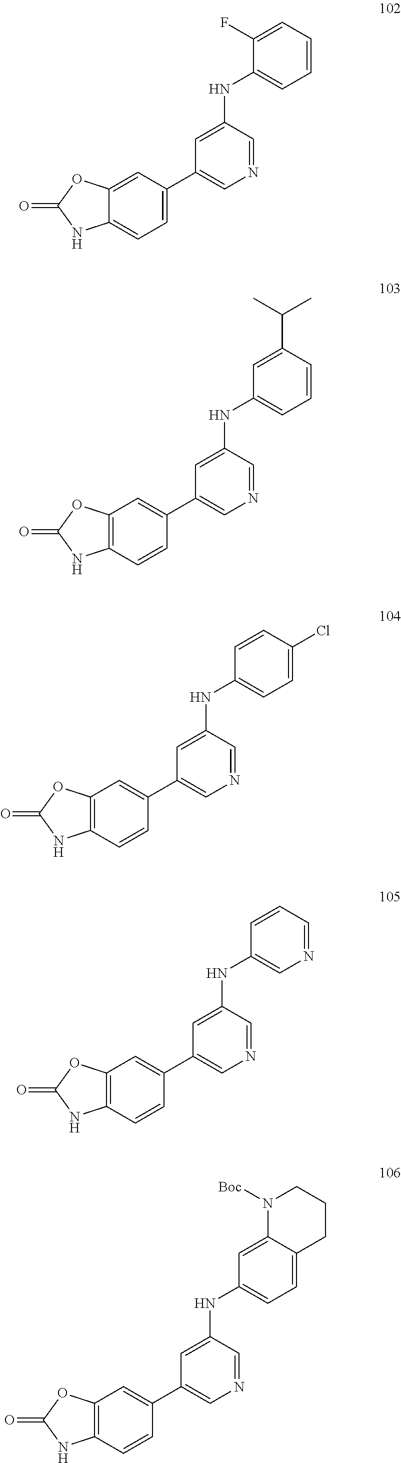 [SMILES: O=c1[nH]c2ccc(-c3cncc(Nc4ccccc4F)c3)cc2o1 ; CC(C)c1cccc(Nc2cncc(-c3ccc4[nH]c(=O)oc4c3)c2)c1 ; O=c1[nH]c2ccc(-c3cncc(Nc4ccc(Cl)cc4)c3)cc2o1 ; O=c1[nH]c2ccc(-c3cncc(Nc4cccnc4)c3)cc2o1 ; CC(C)(C)OC(=O)N1CCCc2ccc(Nc3cncc(-c4ccc5[nH]c(=O)oc5c4)c3)cc21] 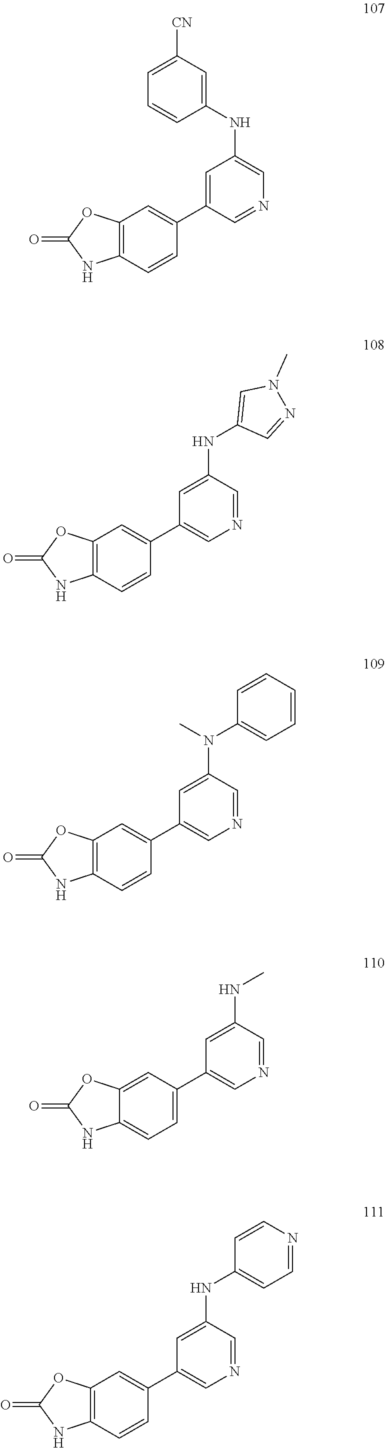 [SMILES: N#Cc1cccc(Nc2cncc(-c3ccc4[nH]c(=O)oc4c3)c2)c1 ; Cn1cc(Nc2cncc(-c3ccc4[nH]c(=O)oc4c3)c2)cn1 ; CN(c1ccccc1)c1cncc(-c2ccc3[nH]c(=O)oc3c2)c1 ; CNc1cncc(-c2ccc3[nH]c(=O)oc3c2)c1 ; O=c1[nH]c2ccc(-c3cncc(Nc4ccncc4)c3)cc2o1] 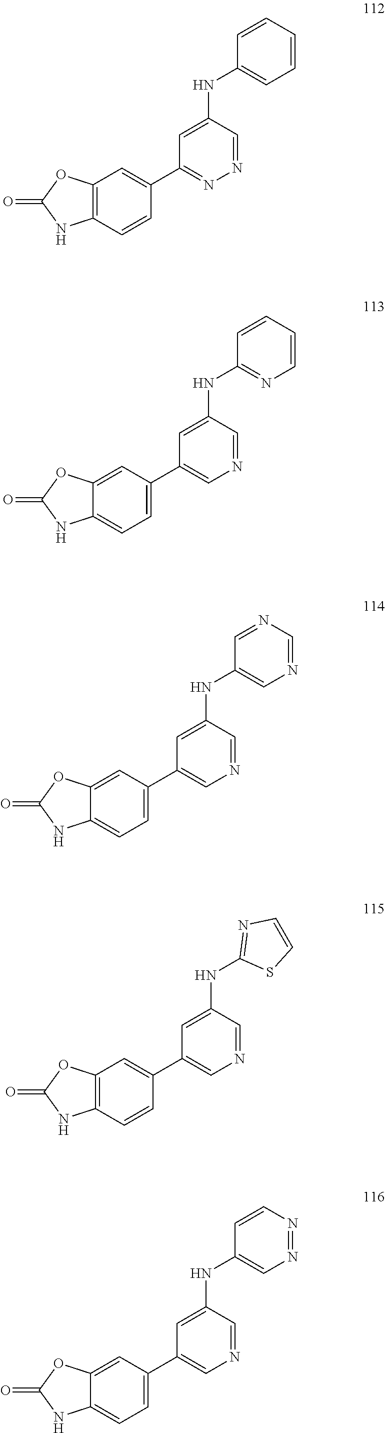 [SMILES: O=c1[nH]c2ccc(-c3cc(Nc4ccccc4)cnn3)cc2o1 ; O=c1[nH]c2ccc(-c3cncc(Nc4ccccn4)c3)cc2o1 ; O=c1[nH]c2ccc(-c3cncc(Nc4cncnc4)c3)cc2o1 ; O=c1[nH]c2ccc(-c3cncc(Nc4nccs4)c3)cc2o1 ; O=c1[nH]c2ccc(-c3cncc(Nc4ccnnc4)c3)cc2o1] 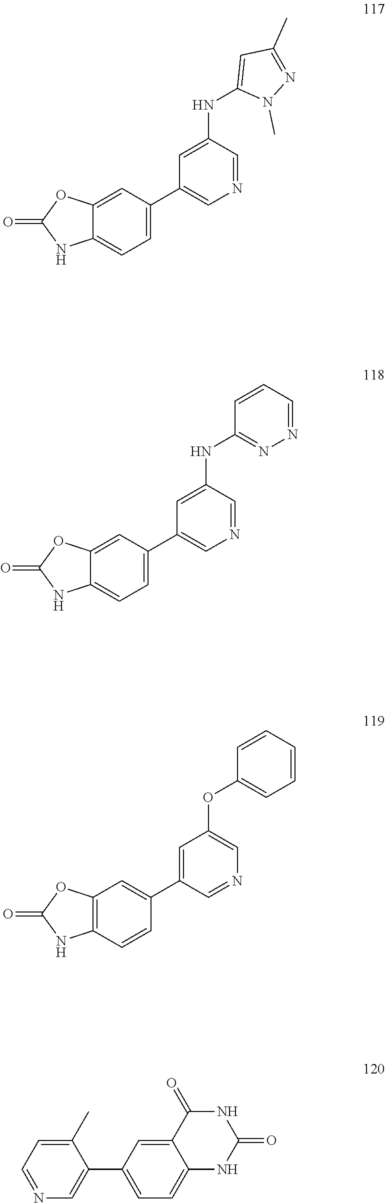 [SMILES: Cc1cc(Nc2cncc(-c3ccc4[nH]c(=O)oc4c3)c2)n(C)n1 ; O=c1[nH]c2ccc(-c3cncc(Nc4cccnn4)c3)cc2o1 ; O=c1[nH]c2ccc(-c3cncc(Oc4ccccc4)c3)cc2o1 ; Cc1ccncc1-c1ccc2[nH]c(=O)[nH]c(=O)c2c1]
a tautomer thereof, a solvate or stereoisomer of the compound or the tautomer, or a pharmaceutically acceptable salt of the foregoing.

44. A pharmaceutical composition comprising a compound according to claim 1, a tautomer thereof, a solvate or stereoisomer of the compound or the tautomer, or a pharmaceutically acceptable salt of the foregoing and at least one pharmaceutically acceptable carrier.

45. A method of treating a disease or condition, comprising administering to a subject in need thereof, a therapeutically effective amount of a compound according to claim 1, a tautomer thereof, a solvate or stereoisomer of the compound or the tautomer, or a pharmaceutically acceptable salt of the foregoing or a pharmaceutical composition comprising the compound according to claim 1, a tautomer thereof, a solvate or stereoisomer of the compound or the tautomer, or a pharmaceutically acceptable salt of the foregoing; wherein the disease or condition is selected from ALS, Parkinson's disease, multiple sclerosis, traumatic brain injury, diabetic neuropathy, and CIPN.

46. A method of treating a disease or condition caused by axonal degeneration, comprising administering to a subject in need thereof, a therapeutically effective amount of a compound according to claim 1, a tautomer thereof, a solvate or stereoisomer of the compound or the tautomer, or a pharmaceutically acceptable salt of the foregoing or a pharmaceutical composition comprising the compound according to claim 1, a tautomer thereof, a solvate or stereoisomer of the compound or the tautomer, or a pharmaceutically acceptable salt of the foregoing.

47. A method of modulating SARM1, comprising contacting a subject in need thereof with a compound according to claim 1, a tautomer thereof, a solvate or stereoisomer of the compound or the tautomer, or a pharmaceutically acceptable salt of the foregoing or a pharmaceutical composition comprising the compound according to claim 1, a tautomer thereof, a solvate or stereoisomer of the compound or the tautomer, or a pharmaceutically acceptable salt of the foregoing.

48. A method of inhibiting or preventing axonal degeneration, comprising contacting a subject in need thereof with a compound according to claim 1, a tautomer thereof, a solvate or stereoisomer of the compound or the tautomer, or a pharmaceutically acceptable salt of the foregoing or a pharmaceutical composition comprising the compound according to claim 1, a tautomer thereof, a solvate or stereoisomer of the compound or the tautomer, or a pharmaceutically acceptable salt of the foregoing.
